(19) United States
(12) Patent Application Publication
(10) Pub. No.: US 2025/0066799 A1
(43) Pub. Date: Feb. 27, 2025

(54) UNNATURAL BASE PAIR COMPOSITIONS AND METHODS OF USE

(71) Applicant: THE SCRIPPS RESEARCH INSTITUTE, La Jolla, CA (US)

(72) Inventors: Floyd E. ROMESBERG, La Jolla, CA (US); Michael P. LEDBETTER, Jamaica Plain, MA (US); Rebekah J. KARADEEMA, San Diego, CA (US)

(21) Appl. No.: 18/947,037

(22) Filed: Nov. 14, 2024

Related U.S. Application Data
(62) Division of application No. 16/913,226, filed on Jun. 26, 2020, now Pat. No. 12,173,291.
Application No. 16/913,226 is a continuation of application No. PCT/US18/67969, filed on Dec. 28, 2018.
(60) Provisional application No. 62/612,062, filed on Dec. 29, 2017.

(51) Int. Cl. C12N 15/67; C07K 14/405; C12N 9/22; C12N 9/96; C12N 15/113; C12N 15/70; C12P 19/34

(52) U.S. Cl. CPC C12N 15/67; C12N 9/22; C12N 9/96; C12N 15/113; C12N 15/70; C12P 19/34; C07K 14/405; C12N 2310/20; C12N 2310/3125; C12N 2800/80

(57) ABSTRACT

Disclosed herein are methods, cells, engineered microorganisms, and kits for increasing the production of polypeptides comprising one or more unnatural amino acids. Further provided are cells, engineered microorganisms, and kits for increasing the retention of unnatural nucleic acids encoding the unnatural amino acids in an engineered cell, or semi-synthetic organism.

### CROSS-REFERENCE

This application is a divisional of U.S. application Ser. No. 16/913,226, filed Jun. 26, 2020, which is a continuation of International Application No. PCT/US2018/067969, filed Dec. 28, 2018, which claims the benefit of U.S. Provisional Application No. 62/612,062, filed on Dec. 29, 2017, each of which are herein incorporated by reference in their entireties.

### STATEMENT AS TO FEDERALLY SPONSORED RESEARCH

This invention was made with government support under Contract No. R35 GM118178/GM/NIGMS awarded by The National Institutes of Health (NIH). The government has certain rights in the invention.

### SEQUENCE LISTING

The instant application contains a Sequence Listing which has been submitted electronically in XML format and is hereby incorporated by reference in its entirety. Said XML copy, created on Oct. 28, 2024, is named “01183-5007-01US-SYN.xml” and is 81,376 bytes in size.

### BACKGROUND OF THE INVENTION

Applications of the ability to sequence-specifically synthesize/amplify oligonucleotides (DNA or RNA) with polymerases are restricted by the limited chemical/physical diversity present in the natural genetic alphabet (the four natural nucleotides A, C, G, and T in DNA, and the four natural nucleotides A, C, G, and U in RNA). An expanded genetic alphabet including unnatural nucleic acids increases the information that can be stored in a cell and facilitate the creating of semi-synthetic organisms (SSOs) that use this increased information to create novel forms of gene expression products.

### SUMMARY OF THE INVENTION

Described herein, in certain embodiments, are methods, cells, engineered microorganisms, plasmids, and kits for increased production of a nucleic acid molecule that comprises an unnatural nucleotide. In some embodiments, also described herein include cells, engineered microorganisms, plasmids, and methods of use that utilizes a a modified transposition-associated protein, a modified DNA repair protein, or a combination thereof for increased production of a nucleic acid molecule that comprises an unnatural nucleotide.

Aspects disclosed herein provide engineered host cells comprising: a first nucleic acid molecule comprising an unnatural nucleotide; and optionally, a second nucleic acid molecule encoding a modified transposition-associated protein or transposable element. In some embodiments, the engineered host cell further comprises a third nucleic acid molecule encoding a modified nucleoside triphosphate transporter, wherein the third nucleic acid molecule is incorporated in a genomic sequence of the engineer host cell, or comprises a plasmid encoding the modified nucleoside triphosphate transporter. In some embodiments, the modified nucleoside triphosphate transporter exhibits increased stability of expression in the engineered host cell as compared to an expression in an equivalent engineered host cell that does not comprise the second nucleic acid molecule encoding the modified transposition-associated protein. In some embodiments, the modified nucleoside triphosphate transporter comprises a deletion of an entire nucleic acid molecule encoding the nucleoside triphosphate transporter, an N-terminal truncation, a C-terminal truncation, or a truncation of both termini. In some embodiments, the modified nucleoside triphosphate transporter comprises a nucleoside triphosphate transporter from Phaeodactylum tricornutum (PtNTT₂). In some embodiments, the modified nucleoside triphosphate transporter comprises a deletion. In some embodiments, the deletion is a terminal deletion or an internal deletion. In some embodiments, the deletion is an N-terminal truncation, a C-terminal truncation, or a truncation of both termini. In some embodiments, the modified nucleoside triphosphate transporter comprises a deletion of about 5, 10, 15, 20, 22, 25, 30, 40, 44, 50, 60, 66, 70, or more amino acid residues. In some embodiments, the modified nucleoside triphosphate transporter comprises a deletion of about 5, 10, 15, 20, 22, 25, 30, 40, 44, 50, 60, 66, 70, or more amino acid residues at the N-terminus. In some embodiments, the modified nucleoside triphosphate transporter comprises a deletion of about 66 amino acid residues at the N-terminus. In some embodiments, the PtNTT₂ is under the control of a promoter selected from a pSC plasmid or a promoter from a lac operon. In some embodiments, the engineered host cell further comprises a Cas9 polypeptide or variants thereof, and a single guide RNA (sgRNA) comprising a crRNA-tracrRNA scaffold, wherein the combination of Cas9 polypeptide or variants thereof and sgRNA modulates replication of the first nucleic acid molecule encoding the unnatural nucleotide. In some embodiments, the sgRNA comprises a target motif that recognizes a modification at the unnatural nucleotide position within the nucleic acid molecule. In some embodiments, the sgRNA further comprises a protospacer adjacent motif (PAM) recognition element. In some embodiments, the PAM element is adjacent to the 3′ terminus of the target motif. In some embodiments, the target motif is between 15 to 30 nucleotides in length. In some embodiments, the combination of Cas9 polypeptide or variants thereof and sgRNA decreases the replication rate of the nucleic acid molecule comprising the modification by about 80%, 85%, 95%, 99%, or higher. In some embodiments, the Cas9 polypeptide is a wild-type Cas9. In some embodiments, the second nucleic acid molecule comprises a gene comprising catalase (cat), IS1 protein insB-4 (insB-4), IS1 protein insA-4 (insA-4), or a combination thereof. In some embodiments, the modified transposition-associated protein comprises Insertion element IS1.4 protein InsB, Insertion element IS1 4 protein InsA, or a combination thereof; and wherein the modified transposable element comprises IS1. In some embodiments, the gene comprises one or more deletions, wherein the one or more deletions comprise an N-terminal deletion, a C-terminal deletion, a truncation at both termini, an internal deletion, and/or a deletion of the entire gene. In some embodiments, the engineered host cell further comprises a fifth nucleic acid molecule encoding a modified DNA repair response-associated protein, wherein the DNA repair response comprises recombinational repair, SOS response, nucleotide excision repair, or methyl-directed mismatch repair, or a combination thereof. In some embodiments, the modified DNA repair response-associated protein comprises RecA, Rad51, RadA, or LexA, or a combination thereof. In some embodiments, the engineered host cell is a prokaryotic cell comprising an Escherichia coli cell, Escherichia coli BL21 (DE3) cell. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of:

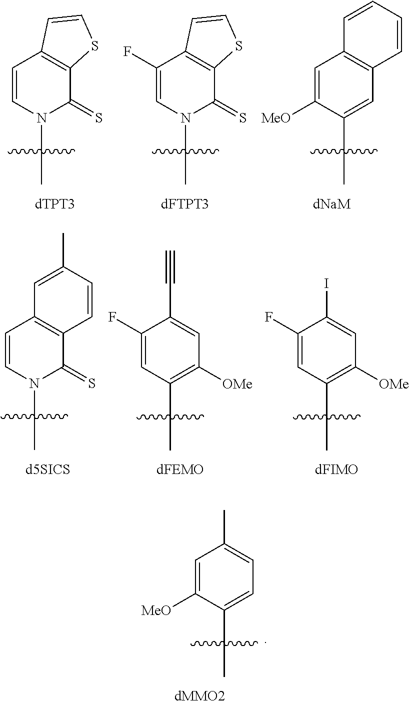

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the engineered host cell further comprises a polymerase. In some embodiments, the polymerase is constitutively expressed. In some embodiments, the polymerase is overexpressed. In some embodiments, the polymerase is a DNA polymerase. In some embodiments, the DNA polymerase is DNA polymerase II. In some embodiments, the polymerase is encoded by the polB gene. In some embodiments, the polB gene is derepressed. In some embodiments, the polB gene is derepressed through integration over an operator half site. In some embodiments, the operator is a lexA operator. In some embodiments, the polymerase is DNA polymerase I. In some embodiments, the polymerase is encoded by the polA gene. In some embodiments, the polymerase is DNA polymerase III. In some embodiments, the polymerase is encoded by the dnaQ gene.

Aspects disclosed herein provide methods of increasing the production of a nucleic acid molecule comprising an unnatural nucleotide, comprising: incubating an engineered host cell with a plurality of unnatural nucleotides, wherein the engineered host cell comprises a modified nucleoside triphosphate transporter and optionally, a modified transposition-associated protein or transposable element; and incorporating the plurality of unnatural nucleotides into one or more newly synthesized DNA strands, thereby generating the unnatural nucleic acid molecule; wherein the modified transposition-associated protein or transposable element and the modified nucleoside triphosphate transporter increases retention of unnatural base pairs comprising the unnatural nucleotides in the one or more newly synthesized DNA strands. In some embodiments, the modified transposition-associated protein comprises Insertion element IS1 4 protein InsB, Insertion element IS1 4 protein InsA, or a combination thereof, and wherein the modified transposable element comprises IS1. In some embodiments, the modified nucleoside triphosphate transporter comprises a codon optimized nucleoside triphosphate transporter from Phaeodactylum tricornutum (PtNTT₂). In some embodiments, the modified nucleoside triphosphate transporter comprises a deletion. In some embodiments, the deletion is a terminal deletion or an internal deletion. In some embodiments, the deletion is an N-terminal truncation, a C-terminal truncation, or a truncation of both termini. In some embodiments, the modified nucleoside triphosphate transporter comprises a deletion of about 5, 10, 15, 20, 22, 25, 30, 40, 44, 50, 60, 66, 70, or more amino acid residues. In some embodiments, the modified nucleoside triphosphate transporter comprises a deletion of about 5, 10, 15, 20, 22, 25, 30, 40, 44, 50, 60, 66, 70, or more amino acid residues at the N-terminus. In some embodiments, the modified nucleoside triphosphate transporter comprises a deletion of about 66 amino acid residues at the N-terminus. In some embodiments, the engineered host cell further comprises a Cas9 polypeptide or variants thereof; and a single guide RNA (sgRNA) comprising a crRNA-tracrRNA scaffold, wherein the combination of Cas9 polypeptide or variants thereof and sgRNA modulates replication of the first nucleic acid molecule encoding the unnatural nucleotide. In some embodiments, the sgRNA comprises a target motif that recognizes a modification at the unnatural nucleotide position within the nucleic acid molecule. In some embodiments, the sgRNA further comprises a protospacer adjacent motif (PAM) recognition element. In some embodiments, the PAM element is adjacent to the 3′ terminus of the target motif. In some embodiments, the target motif is between 15 to 30 nucleotides in length. In some embodiments, the combination of Cas9 polypeptide or variants thereof and sgRNA decreases the replication rate of the nucleic acid molecule comprising the modification by about 80%, 85%, 95%, 99%, or higher. In some embodiments, the Cas9 polypeptide is a wild-type Cas9. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of:

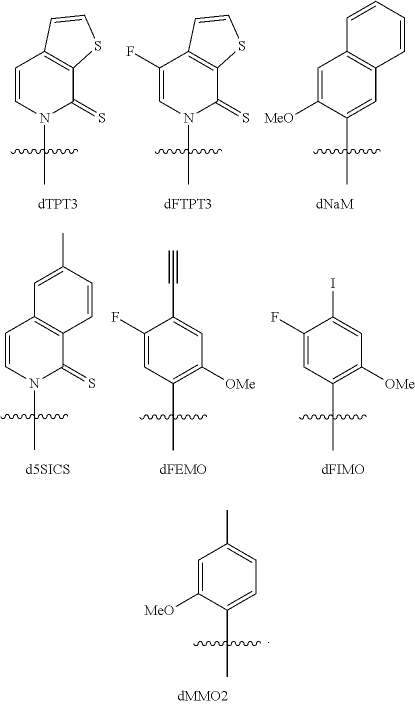

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the engineered host cell further comprises a polymerase. In some embodiments, the polymerase is constitutively expressed. In some embodiments, the polymerase is overexpressed. In some embodiments, the polymerase is a DNA polymerase. In some embodiments, the DNA polymerase is DNA polymerase II. In some embodiments, the polymerase is encoded by the polB gene. In some embodiments, the polB gene is derepressed. In some embodiments, the polB gene is derepressed through integration over an operator half site. In some embodiments, the operator is a lexA operator. In some embodiments, the polymerase is DNA polymerase I. In some embodiments, the polymerase is encoded by the polA gene. In some embodiments, the polymerase is DNA polymerase III. In some embodiments, the polymerase is encoded by the dnaQ gene.

Aspects disclosed herein provide methods of preparing a modified polypeptide comprising an unnatural amino acid, comprising: incubating an engineered host cell with a plurality of unnatural nucleotides, wherein the engineered host cell comprises a modified nucleoside triphosphate transporter and, optionally, a modified transposition-associated protein or transposable element; and incorporating the plurality of unnatural nucleotides into one or more newly synthesized DNA strands, thereby generating the unnatural nucleic acid molecule; wherein the modified transposition-associated protein or transposable element and the modified nucleoside triphosphate transporter increases retention of unnatural base pairs which facilitates incorporation of the plurality of unnatural nucleotides into the newly synthesized polypeptide to generate the modified polypeptide. In some embodiments, the modified transposition-associated protein comprises Insertion element IS1 4 protein InsB, Insertion element IS1 4 protein InsA, or a combination thereof, and wherein the modified transposable element comprises IS1. In some embodiments, the modified nucleoside triphosphate transporter comprises a codon optimized nucleoside triphosphate transporter from Phaeodactylum tricornutum (PtNTT₂). In some embodiments, the modified nucleoside triphosphate transporter comprises a deletion. In some embodiments, the deletion is a terminal deletion or an internal deletion. In some embodiments, the deletion is an N-terminal truncation, a C-terminal truncation, or a truncation of both termini. In some embodiments, the modified nucleoside triphosphate transporter comprises a deletion of about 5, 10, 15, 20, 22, 25, 30, 40, 44, 50, 60, 66, 70, or more amino acid residues. In some embodiments, the modified nucleoside triphosphate transporter comprises a deletion of about 5, 10, 15, 20, 22, 25, 30, 40, 44, 50, 60, 66, 70, or more amino acid residues at the N-terminus. In some embodiments, the modified nucleoside triphosphate transporter comprises a deletion of about 66 amino acid residues at the N-terminus. In some embodiments, the engineered host cell further comprises a Cas9 polypeptide or variants thereof; and a single guide RNA (sgRNA) comprising a crRNA-tracrRNA scaffold, wherein the combination of Cas9 polypeptide or variants thereof and sgRNA modulates replication of the first nucleic acid molecule encoding the unnatural nucleotide. In some embodiments, the sgRNA comprises a target motif that recognizes a modification at the unnatural nucleotide position within the nucleic acid molecule. In some embodiments, the sgRNA further comprises a protospacer adjacent motif (PAM) recognition element. In some embodiments, the PAM element is adjacent to the 3′ terminus of the target motif. In some embodiments, the target motif is between 15 to 30 nucleotides in length. In some embodiments, the combination of Cas9 polypeptide or variants thereof and sgRNA decreases the replication rate of the nucleic acid molecule comprising the modification by about 80%, 85%, 95%, 99%, or higher. In some embodiments, the Cas9 polypeptide is a wild-type Cas9. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N₄-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of:

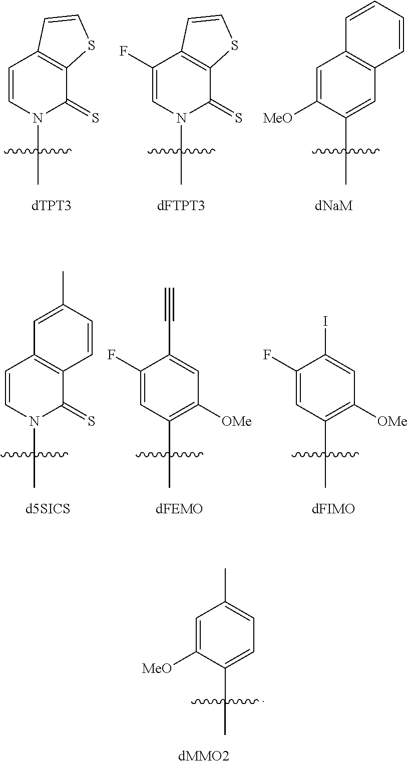

In some embodiments, the unnatural nucleotide further comprises an unnatural sugar moiety selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the engineered host cell further comprises a polymerase. In some embodiments, the polymerase is constitutively expressed. In some embodiments, the polymerase is overexpressed. In some embodiments, the polymerase is a DNA polymerase. In some embodiments, the DNA polymerase is DNA polymerase II. In some embodiments, the polymerase is encoded by the polB gene. In some embodiments, the polB gene is derepressed. In some embodiments, the polB gene is derepressed through integration over an operator half site. In some embodiments, the operator is a lexA operator. In some embodiments, the polymerase is DNA polymerase I. In some embodiments, the polymerase is encoded by the polA gene. In some embodiments, the polymerase is DNA polymerase III. In some embodiments, the polymerase is encoded by the dnaQ gene.

Aspects disclosed herein provide engineered host cells for producing an unnatural product comprising a modified DNA repair response-associated protein. In some embodiments, the DNA repair response comprises recombinational repair. In some embodiments, the DNA repair response comprises SOS response. In some embodiments, the engineered host cell is a prokaryotic cell, a eukaryotic cell, or a yeast cell. In some embodiments, the engineered host cell is a prokaryotic cell. In some embodiments, the prokaryotic cell is an Escherichia coli cell. In some embodiments, the Escherichia coli cell is an Escherichia coli BL21 (DE3) cell. In some embodiments, the modified DNA repair response-associated protein is RecA. In some embodiments, the engineered host cell is engineered to express a gene encoding RecA. In some embodiments, the modified DNA repair response-associated protein is Rad51. In some embodiments, the engineered host cell is engineered to express a gene encoding Rad51. In some embodiments, the modified DNA repair response-associated protein is RadA. In some embodiments, the modified DNA repair response-associated protein is LexA. In some embodiments, the gene encoding the modified DNA repair response-associated protein comprises one or more mutations, one or more deletions, or a combination thereof. In some embodiments, the gene comprises an N-terminal deletion, a C-terminal deletion, a truncation at both termini, or an internal deletion. In some embodiments, recA, rad51, and/or radA comprises one or more mutations, one or more deletions, or a combination thereof. In some embodiments, recA, rad51, and radA each independently comprises an N-terminal deletion, a C-terminal deletion, a truncation at both termini, or an internal deletion. In some embodiments, recA comprises an N-terminal deletion, a C-terminal deletion, a truncation at both termini, or an internal deletion. In some embodiments, recA comprises an internal deletion of residues 2-347. In some embodiments, lexA comprises one or more mutations, one or more deletions, or a combination thereof. In some embodiments, lexA comprises a mutation at amino acid position S119, optionally a S119A mutation. In some embodiments, the engineered host cell further comprises a polymerase. In some embodiments, the polymerase is constitutively expressed. In some embodiments, the polymerase is overexpressed. In some embodiments, the polymerase is a DNA polymerase. In some embodiments, the DNA polymerase is DNA polymerase II. In some embodiments, the polymerase is encoded by the polB gene. In some embodiments, the polB gene is derepressed. In some embodiments, the polB gene is derepressed through integration over an operator half site. In some embodiments, the operator is a lexA operator. In some embodiments, the polymerase is DNA polymerase I. In some embodiments, the polymerase is encoded by the polA gene. In some embodiments, the polymerase is DNA polymerase III. In some embodiments, the polymerase is encoded by the dnaQ gene.

Aspects disclosed herein provide engineered host cells for producing an unnatural product comprising a modified DNA repair response-associated protein and a polymerase, wherein the polymerase has an elevated expression relative to an equivalent host cell comprising an equivalent polymerase with a basal expression level. In some embodiments, the DNA repair response comprises recombinational repair. In some embodiments, the DNA repair response comprises SOS response. In some embodiments, the polymerase is constitutively expressed. In some embodiments, the polymerase is DNA polymerase II. In some embodiments, the DNA repair response comprises recombinational repair, SOS response, nucleotide excision repair, or methyl-directed mismatch repair. In some embodiments, the DNA repair response comprises recombinational repair. In some embodiments, the DNA repair response comprises SOS response. In some embodiments, the engineered host cell is a prokaryotic cell, a eukaryotic cell, or a yeast cell. In some embodiments, the engineered host cell is a prokaryotic cell. In some embodiments, the prokaryotic cell is an Escherichia coli cell. In some embodiments, the Escherichia coli cell is an Escherichia coli BL21 (DE3) cell. In some embodiments, the modified DNA repair response-associated protein is RecA. In some embodiments, the modified DNA repair response-associated protein is Rad51. In some embodiments, the modified DNA repair response-associated protein is RadA. In some embodiments, the modified DNA repair response-associated protein is LexA. In some embodiments, the gene encoding the defective protein comprises one or more mutations, one or more deletions, or a combination thereof. In some embodiments, the gene comprises an N-terminal deletion, a C-terminal deletion, a truncation at both termini, or an internal deletion. In some embodiments, recA, rad51, and/or radA comprises one or more mutations, one or more deletions, or a combination thereof. In some embodiments, recA, rad51, and radA each independently comprises an N-terminal deletion, a C-terminal deletion, a truncation at both termini, or an internal deletion. In some embodiments, recA comprises an N-terminal deletion, a C-terminal deletion, a truncation at both termini, or an internal deletion. In some embodiments, recA comprises an internal deletion of residues 2-347. In some embodiments, lexA comprises one or more mutations, one or more deletions, or a combination thereof. In some embodiments, lexA comprises a mutation at amino acid position S119, optionally a S119A mutation. In some embodiments, the engineered host cell further comprises a nucleoside triphosphate transporter from Phaeodactylum tricornutum (PtNTT₂). In some embodiments, the nucleoside triphosphate transporter from PtNTT₂ is modified. In some embodiments, the modified nucleoside triphosphate transporter is encoded by a nucleic acid molecule. In some embodiments, the nucleic acid molecule encoding the modified nucleoside triphosphate transporter is incorporated in the genomic sequence of the engineered host cell. In some embodiments, the engineered host cell comprises a plasmid comprising the nucleic acid molecule encoding the modified nucleoside triphosphate transporter. In some embodiments, the modified nucleoside triphosphate transporter is a codon optimized nucleoside triphosphate transporter from Phaeodactylum tricornutum. In some embodiments, the modified nucleoside triphosphate transporter comprises a deletion. In some embodiments, the deletion is a terminal deletion or an internal deletion. In some embodiments, the deletion is an N-terminal truncation, a C-terminal truncation, or a truncation of both termini. In some embodiments, the modified nucleoside triphosphate transporter comprises a deletion of about 5, 10, 15, 20, 22, 25, 30, 40, 44, 50, 60, 66, 70, or more amino acid residues. In some embodiments, the modified nucleoside triphosphate transporter comprises a deletion of about 5, 10, 15, 20, 22, 25, 30, 40, 44, 50, 60, 66, 70, or more amino acid residues at the N-terminus. In some embodiments, the modified nucleoside triphosphate transporter comprises a deletion of about 66 amino acid residues at the N-terminus. In some embodiments, the modified nucleoside triphosphate transporter is under the control of a promoter selected from a pSC plasmid or a promoter from a lac operon. In some embodiments, the lac operon is an E. coli lac operon. In some embodiments, the lac operon is selected from P_{bla}, P_{lac}, P_{lacUV5}, P_{H207}, P_{λ}, P_{tac}, or P_{N25}. In some embodiments, the modified nucleoside triphosphate transporter is under the control of promoter P_{lacUV5}. In some embodiments, the engineered host cell further comprises a Cas9 polypeptide or variants thereof, and a single guide RNA (sgRNA) comprising a crRNA-tracrRNA scaffold, wherein the combination of Cas9 polypeptide or variants thereof and sgRNA modulates replication of a nucleic acid molecule comprising an unnatural nucleotide. In some embodiments, the sgRNA comprises a target motif that recognizes a modification at the unnatural nucleotide position within the nucleic acid molecule. In some embodiments, the sgRNA further comprises a protospacer adjacent motif (PAM) recognition element. In some embodiments, the PAM element is adjacent to the 3′ terminus of the target motif. In some embodiments, the target motif is between 15 to 30 nucleotides in length. In some embodiments, the combination of Cas9 polypeptide or variants thereof and sgRNA decreases the replication rate of the nucleic acid molecule comprising the modification by about 80%, 85%, 95%, 99%, or higher. In some embodiments, the Cas9 polypeptide is a wild-type Cas9. In some embodiments, the engineered host cell further comprises an unnatural nucleotide. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

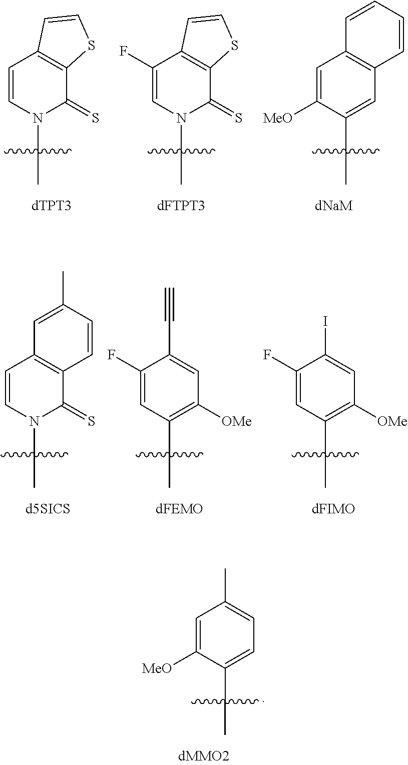

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural base is selected from the group consisting of

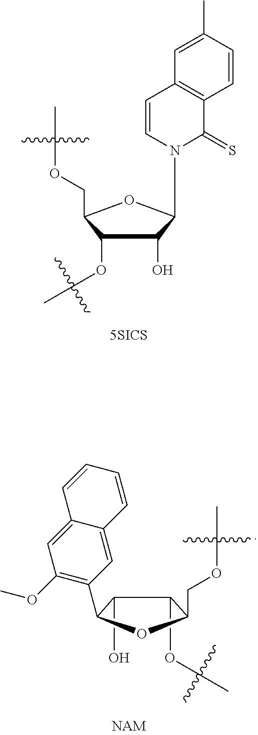

In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriesters, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the unnatural nucleotide is dNaMTP and/or dTPT₃TP. In some embodiments, the unnatural nucleotide is integrated into the engineered host cell genome. In some embodiments, the unnatural nucleotide is integrated into a chromosome. In some embodiments, the unnatural nucleotide is integrated into an arsB locus. In some embodiments, the engineered host cell enables unnatural base pair retention of about 50%, 55%, 60%, 65%, 70%, 75%, 80%, 85%, 90%, 91%, 92%, 93%, 94%, 95%, 96%, 97%, 98%, 99% or more, relative to an equivalent engineered host cell in the absence of the modified DNA repair response-associated protein or in the absence of the modified DNA repair response-associated protein in combination with the overexpressed polymerase. In some embodiments, the engineered host cell enables unnatural base pair retention at least 50% after more than 50, more than 100, more than 120, more than 130, more than 150, or more than 200 generations. In some embodiments, the engineered host cell enables unnatural base pair retention at least 55% after more than 50, more than 100, more than 120, more than 130, more than 137, more than 150, or more than 200 generations. In some embodiments, the unnatural product is a nucleic acid molecule comprising an unnatural nucleotide. In some embodiments, the unnatural product is a polypeptide comprising an unnatural amino acid. In some embodiments, the engineered host cell is a semi-synthetic organism.

Aspects disclosed herein provide nucleic acid molecules comprising an unnatural nucleotide produced by an engineered host cell described herein.

Aspects disclosed herein provide polypeptide comprising one or more unnatural amino acids produced by an engineered host cell described herein.

Aspects disclosed herein provide methods of increasing fidelity of replication of a nucleic acid molecule comprising an unnatural nucleotide, comprising: (a) incubating an engineered host cell described herein with a plurality of unnatural nucleotides; and (b) incorporating the plurality of unnatural nucleotides into one or more newly synthesized DNA strands, thereby generating the unnatural nucleic acid molecule; wherein the modified DNA repair response-associated protein and optionally the overexpressed polymerase increases fidelity of replication of the unnatural base pairs comprising the unnatural nucleotides in the one or more newly synthesized DNA strands. In some embodiments, the DNA repair response comprises recombinational repair. In some embodiments, the DNA repair response comprises SOS response. In some embodiments, the increased production of the nucleic acid molecule comprising an unnatural nucleotide is relative to the production of the nucleic acid molecule in an equivalent host cell in the absence of the modified DNA repair response-associated protein and optionally the overexpressed polymerase. In some embodiments, the increased production of the nucleic acid molecule is at least 5%, 10%, 20%, 30%, 40%, 50%, 60%, 70%, 80%, 90%, or 99% higher than the production of the nucleic acid molecule in an equivalent host cell in the absence of the modified DNA repair response-associated protein and optionally the overexpressed polymerase. In some embodiments, the increased production of the nucleic acid molecule is more than 1-fold, 2-fold, 3-fold, 4-fold, 5-fold, 10-fold, 15-fold, 20-fold, 25-fold, 30-fold, 40-fold, 50-fold, 100-fold, or higher than the production of the nucleic acid molecule in an equivalent host cell in the absence of the modified DNA repair response-associated protein and optionally the overexpressed polymerase. In some embodiments, the increased production of the nucleic acid molecule is from 1-fold to 5-fold, from 5-fold to 10-fold, from 10-fold to 15-fold, from 15-fold to 20-fold, from 20-fold to 25-fold, from 25-fold to 30-fold, from 30-fold to 40-fold, from 40-fold to 50-fold, from 50-fold to 60-fold, from 60-fold to 70-fold, from 70-fold to 80-fold, from 80-fold to 90-fold, from 90-fold to 100-fold, or from 100-fold to 200-fold higher than the production of the nucleic acid molecule in an equivalent host cell in the absence of the modified DNA repair response-associated protein and optionally the overexpressed polymerase. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N₄-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N₆-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N₆-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

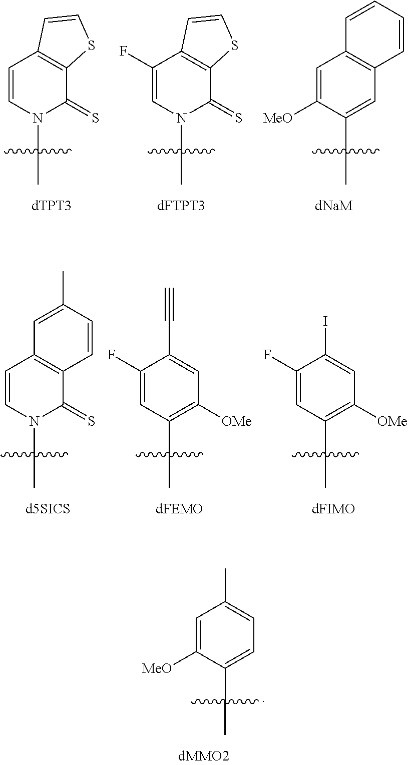

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural base is selected from the group consisting of

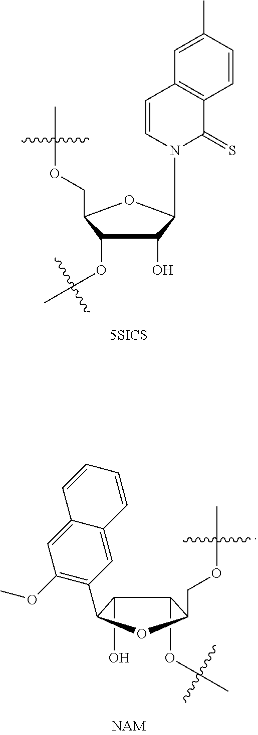

In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the unnatural nucleotide is dNaMTP and/or dTPT₃TP. In some embodiments, the unnatural nucleotide is integrated into the engineered host cell genome. In some embodiments, the unnatural nucleotide is integrated into a chromosome. In some embodiments, the unnatural nucleotide is integrated into an arsB locus. In some embodiments, the modified DNA repair response-associated protein is RecA. In some embodiments, the modified DNA repair response-associated protein is Rad51. In some embodiments, the modified DNA repair response-associated protein is RadA. In some embodiments, the modified DNA repair response-associated protein is LexA. In some embodiments, the gene encoding the modified DNA repair response-associated protein comprises one or more mutations, one or more deletions, or a combination thereof. In some embodiments, the gene comprises an N-terminal deletion, a C-terminal deletion, a truncation at both termini, or an internal deletion. In some embodiments, recA, rad51, and/or radA comprises one or more mutations, one or more deletions, or a combination thereof. In some embodiments, recA, rad51, and radA each independently comprises an N-terminal deletion, a C-terminal deletion, a truncation at both termini, or an internal deletion. In some embodiments, recA comprises an N-terminal deletion, a C-terminal deletion, a truncation at both termini, or an internal deletion. In some embodiments, recA comprises an internal deletion of residues 2-347. In some embodiments, lexA comprises one or more mutations, one or more deletions, or a combination thereof. In some embodiments, lexA comprises a mutation at amino acid position S119, optionally a S119A mutation.

Aspects disclosed herein provide methods of increasing the production of a nucleic acid molecule comprising an unnatural nucleotide, comprising: (a) incubating an engineered host cell described herein with a plurality of unnatural nucleotides; and (b) incorporating the plurality of unnatural nucleotides into one or more newly synthesized DNA strands, thereby generating the unnatural nucleic acid molecule; wherein the modified DNA repair response-associated protein and optionally the overexpressed polymerase increases retention of unnatural base pairs comprising the unnatural nucleotides in the one or more newly synthesized DNA strands. In some embodiments, the DNA repair response comprises recombinational repair. In some embodiments, the DNA repair response comprises SOS response. In some embodiments, the increased production of the nucleic acid molecule comprising an unnatural nucleotide is relative to the production of the nucleic acid molecule in an equivalent host cell in the absence of the modified DNA repair response-associated protein and optionally the overexpressed polymerase. In some embodiments, the increased production of the nucleic acid molecule is at least 5%, 10%, 20%, 30%, 40%, 50%, 60%, 70%, 80%, 90%, or 99% higher than the production of the nucleic acid molecule in an equivalent host cell in the absence of the modified DNA repair response-associated protein and optionally the overexpressed polymerase. In some embodiments, the increased production of the nucleic acid molecule is more than 1-fold, 2-fold, 3-fold, 4-fold, 5-fold, 10-fold, 15-fold, 20-fold, 25-fold, 30-fold, 40-fold, 50-fold, 100-fold, or higher than the production of the nucleic acid molecule in an equivalent host cell in the absence of the modified DNA repair response-associated protein and optionally the overexpressed polymerase. In some embodiments, the increased production of the nucleic acid molecule is from 1-fold to 5-fold, from 5-fold to 10-fold, from 10-fold to 15-fold, from 15-fold to 20-fold, from 20-fold to 25-fold, from 25-fold to 30-fold, from 30-fold to 40-fold, from 40-fold to 50-fold, from 50-fold to 60-fold, from 60-fold to 70-fold, from 70-fold to 80-fold, from 80-fold to 90-fold, from 90-fold to 100-fold, or from 100-fold to 200-fold higher than the production of the nucleic acid molecule in an equivalent host cell in the absence of the modified DNA repair response-associated protein and optionally the overexpressed polymerase. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

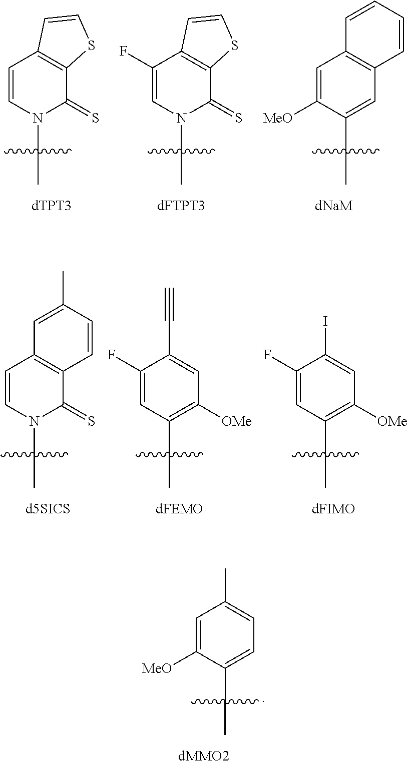

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkyl amino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural base is selected from the group consisting of

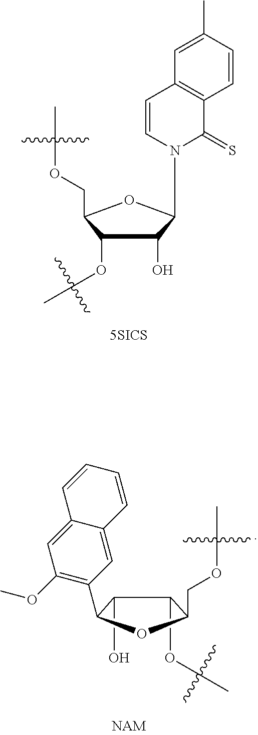

In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the unnatural nucleotide is dNaMTP and/or dTPT₃TP. In some embodiments, the unnatural nucleotide is integrated into the engineered host cell genome. In some embodiments, the unnatural nucleotide is integrated into a chromosome. In some embodiments, the unnatural nucleotide is integrated into an arsB locus. In some embodiments, the modified DNA repair response-associated protein is RecA. In some embodiments, the modified DNA repair response-associated protein is Rad51. In some embodiments, the modified DNA repair response-associated protein is RadA. In some embodiments, the modified DNA repair response-associated protein is LexA. In some embodiments, the gene encoding the modified DNA repair response-associated protein comprises one or more mutations, one or more deletions, or a combination thereof. In some embodiments, the gene comprises an N-terminal deletion, a C-terminal deletion, a truncation at both termini, or an internal deletion. In some embodiments, recA, rad51, and/or radA comprises one or more mutations, one or more deletions, or a combination thereof. In some embodiments, recA, rad51, and radA each independently comprises an N-terminal deletion, a C-terminal deletion, a truncation at both termini, or an internal deletion. In some embodiments, recA comprises an N-terminal deletion, a C-terminal deletion, a truncation at both termini, or an internal deletion. In some embodiments, recA comprises an internal deletion of residues 2-347. In some embodiments, lexA comprises one or more mutations, one or more deletions, or a combination thereof. In some embodiments, lexA comprises a mutation at amino acid position S119, optionally a S119A mutation.

Aspects disclosed herein provide methods of preparing a modified polypeptide comprising an unnatural amino acid, comprising: (a) incubating an engineered host cell described herein with a plurality of unnatural amino acids; and (b) incorporating the plurality of unnatural amino acids into a newly synthesized polypeptide, thereby generating the modified polypeptide; wherein the modified DNA repair response-associated protein and optionally the overexpressed polymerase increases retention of unnatural base pairs which facilitates incorporation of the plurality of unnatural amino acids into the newly synthesized polypeptide to generate the modified polypeptide. In some embodiments, the DNA repair response comprises recombinational repair. In some embodiments, the DNA repair response comprises SOS response. In some embodiments, the modified polypeptide is further conjugated with a conjugating moiety to generate a modified polypeptide conjugate. In some embodiments, the conjugating moiety is a protein or binding fragment thereof, a polymer, a therapeutic agent, an imaging agent, or a combination thereof. In some embodiments, the modified polypeptide is further conjugated with a therapeutic agent. In some embodiments, the modified polypeptide is an imaging agent. In some embodiments, the modified polypeptide conjugate is further formulated with a pharmaceutical excipient to generate a pharmaceutical composition. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

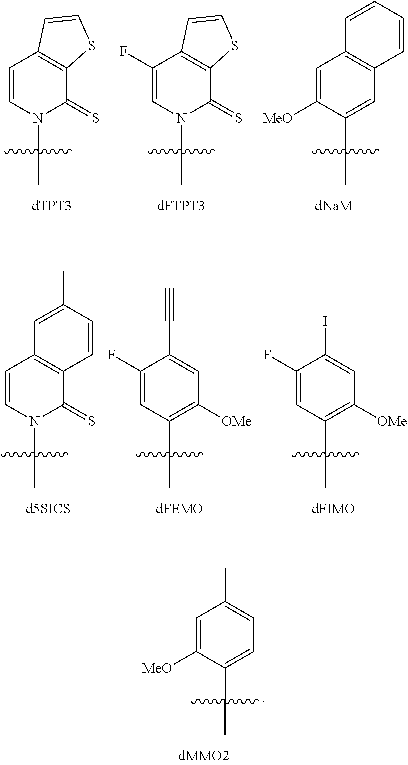

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural base is selected from the group consisting of

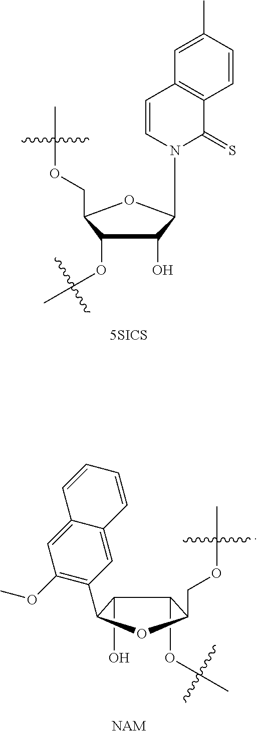

In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the unnatural nucleotide is dNaMTP and/or dTPT₃TP. In some embodiments, the unnatural nucleotide is integrated into the engineered host cell genome. In some embodiments, the unnatural nucleotide is integrated into a chromosome. In some embodiments, the unnatural nucleotide is integrated into an arsB locus. In some embodiments, the modified DNA repair response-associated protein is RecA. In some embodiments, the modified DNA repair response-associated protein is Rad51. In some embodiments, the modified DNA repair response-associated protein is RadA. In some embodiments, the modified DNA repair response-associated protein is LexA. In some embodiments, the gene encoding the modified DNA repair response-associated protein comprises one or more mutations, one or more deletions, or a combination thereof. In some embodiments, the gene comprises an N-terminal deletion, a C-terminal deletion, a truncation at both termini, or an internal deletion. In some embodiments, recA, rad51, and/or radA comprises one or more mutations, one or more deletions, or a combination thereof. In some embodiments, recA, rad51, and radA each independently comprises an N-terminal deletion, a C-terminal deletion, a truncation at both termini, or an internal deletion. In some embodiments, recA comprises an N-terminal deletion, a C-terminal deletion, a truncation at both termini, or an internal deletion. In some embodiments, recA comprises an internal deletion of residues 2-347. In some embodiments, lexA comprises one or more mutations, one or more deletions, or a combination thereof. In some embodiments, lexA comprises a mutation at amino acid position S119, optionally a S119A mutation.

Aspects disclosed herein provide methods of treating a disease or condition, comprising administering to a subject in need thereof a pharmaceutical composition comprising a modified polypeptide prepared by the methods disclosed herein, thereby treating the disease or condition.

Aspects disclosed herein provide kits comprising an engineered host cells described herein.

Aspects disclosed herein provide engineered host cells for producing an unnatural product comprising a modified RecA. In some embodiments, a gene encoding the modified RecA comprises one or more mutations, one or more deletions, or a combination thereof. In some embodiments, the gene comprises an N-terminal deletion, a C-terminal deletion, a truncation at both termini, or an internal deletion. In some embodiments, recA comprises an N-terminal deletion, a C-terminal deletion, a truncation at both termini, or an internal deletion. In some embodiments, recA comprises an internal deletion of residues 2-347.

Aspects disclosed herein provide engineered host cells for producing an unnatural product comprising a modified RecA and an overexpressed DNA polymerase II, wherein the expression level of the overexpressed DNA polymerase II is relative to an equivalent host cell comprising an equivalent DNA polymerase II with a basal expression level.

Aspects disclosed herein provide methods of increasing the production of a nucleic acid molecule comprising an unnatural nucleotide, comprising: (a) incubating an engineered host cell with a plurality of unnatural nucleotides, wherein the engineered host cell comprises a modified RecA and optionally an overexpressed DNA polymerase II, and wherein the expression level of the overexpressed DNA polymerase II is relative to an equivalent host cell comprising an equivalent DNA polymerase II with a basal expression level; and (b) incorporating the plurality of unnatural nucleotides into one or more newly synthesized DNA strands, thereby generating the unnatural nucleic acid molecule; wherein the modified DNA repair response-associated protein and optionally the overexpressed polymerase increases retention of unnatural base pairs comprising the unnatural nucleotides in the one or more newly synthesized DNA strands.

Aspects disclosed herein provide methods of preparing a modified polypeptide comprising an unnatural amino acid, comprising: (a) incubating an engineered host cell with a plurality of unnatural amino acids, wherein the engineered host cell comprises a modified RecA and optionally an overexpressed DNA polymerase II, and wherein the expression level of the overexpressed DNA polymerase II is relative to an equivalent host cell comprising an equivalent DNA polymerase II with a basal expression level; and (b)incorporating the plurality of unnatural amino acids into a newly synthesized polypeptide, thereby generating the modified polypeptide; wherein the modified DNA repair response-associated protein and optionally the overexpressed polymerase increases retention of unnatural base pairs which facilitates incorporation of the plurality of unnatural amino acids into the newly synthesized polypeptide to generate the modified polypeptide. In some embodiments, the DNA repair response comprises recombinational repair. In some embodiments, the DNA repair response comprises SOS response. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle.

In some embodiments, the unnatural base is selected from the group consisting of

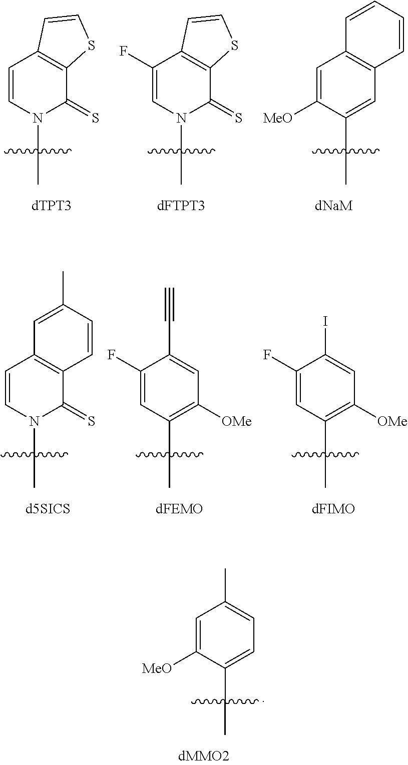

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)nOCH₃, —O(CH₂)n NH₂, —O(CH₂)n CH₃, —O(CH₂)n—ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural base is selected from the group consisting of

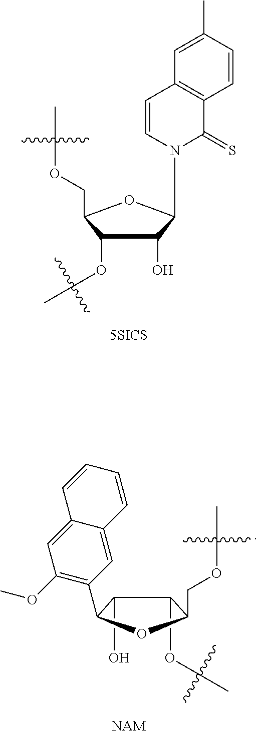

In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the unnatural nucleotide is dNaMTP and/or dTPT₃TP. In some embodiments, the unnatural nucleotide is integrated into the engineered host cell genome. In some embodiments, the unnatural nucleotide is integrated into a chromosome. In some embodiments, the unnatural nucleotide is integrated into an arsB locus.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1E illustrate unnatural base pairs (UBPs) and the contribution of DNA damage and tolerance pathways to its retention. FIG. 1A illustrates the dNaM-dTPT3 UBP and a natural dG-dC base pair. FIG. 1B illustrates the strains deficient for NER (ΔuvrC), MMR (ΔmutH), or RER (ΔrecA). FIG. 1C illustrates strains deficient for RER and SOS (ΔrecA) and strains deficient only for SOS (lexA(S119A)). FIG. 1D illustrates strains deficient for the SOS regulated polymerases Pol II (ΔpolB) or Pols IV and V (ΔdinBΔumuCD) or RER and SOS (ΔrecA). FIG. 1E illustrates strains with Pol I^{exo-}(polA(D424A, K890R)) or Pol III^{exo-}(dnaQ(D12N)) in wild-type, ΔpolB, or ΔpolBΔrecA backgrounds. In each case the indicated strains were challenged with replicating a plasmid with the UBP embedded within the sequence indicated (X=dNaM). n≥3 for all data shown; points represent individual replicates; bars represent sample means; error bars represent S.D.

FIG. 2A-FIG. 2C illustrate replisome reprogramming results in optimized UBP retention. FIG. 2A illustrates that retention of UBP in individual clones of WT-Opt (medium grey), ΔrecA-Opt (dark grey), and Pol II⁺ΔrecA-Opt (light grey) after selection on solid growth media. Each strain was challenged with replicating pINF-borne UBPs in sequence contexts of varying difficulty (GTAXAGA<TCCXCGT<TCCXGGT). Each point represents and individual clone, and n≥12 for each distribution. FIG. 2B illustrates growth curves of chromosomal UBP integrants of WT-Opt (medium grey), ΔrecA-Opt (dark grey), and Pol II⁺ΔrecA-Opt (light grey) cells during exponential phase growth in media with (circles/solid lines) and without (squares/dotted lines) dNaMTP and dTPT3TP. Data is fit with theoretical exponential growth curves. n=3; small points represent individual replicates; large points represent sample means; error bars for time and OD600 represent S.D. FIG. 2C illustrates retention of the chromosomal dNaM-dTPT3 UBP in WT-Opt (medium grey), ΔrecA-Opt (dark grey), and Pol II⁺ΔrecA-Opt (light grey) cells was measured over long-term growth. n=3; small points represent individual replicates; large points represent sample means; error bars represent two S.D. for both cell doublings and retention except for Pol II⁺ΔrecA-Opt data. After approximately seventy doublings, one replicate of Pol II⁺ΔrecA-Opt strain was contaminated with WT-Opt cells. Therefore, data at and after the black arrow represent the mean of only two independent experiments for Pol II⁺ΔrecA-Opt.

FIG. 3 illustrates increased PtNTT2 activity over long-term growth (10 passages) in strains containing a knockout of IS1, as compared to YZ3 strain that has been engineered to constitutively express a modified PtNTT2 nucleotide transporter gene from the chromosomal lacZYA locus.

FIG. 4A-FIG. 4C exemplify PtNTT2 expression constructs. Expression constructs for PtNTT2(66-575) are shown. FIG. 4A shows pACS2 was used to generate all data provided in FIG. 1 except for Pol III^{Exo-}strains. FIG. 4B shows pACS2+dnaQ(D12N) was used to generate Pol IIIexo-strain data. FIG. 4C shows chromosomal expression from the lacZYA locus was used to generate all FIG. 3 data.

FIG. 5 illustrates exonuclease deficient polymerases replicating TCAXAGT pINF replication data for exonuclease deficient polymerase strains is shown. The same strains from FIG. 1E were also tested for their ability to replicate TCAXAGT (X=dNaM). N>≥3 for all data shown; error bars represent 95% empirical bootstrap confidence intervals

FIG. 6A-FIG. 6B illustrate polA(D424A, K890R), P_polB design. Construction strategy of polA(D424A, K890R) and derepressed PpolB are shown. FIG. 6A shows that polA was truncated to its 5″-3″ exonuclease domain (corresponding to PolA(1-341)). Then the desired D424A mutation was introduced. The K890R mutation occurred in PCR and was predicted to have limited impact on Pol I function. FIG. 6B shoes that PpolB was derepressed (PPol II+) through integration over one of the lexA operator half sites (bold) present upstream of the −35 sequence of the promoter.

FIG. 7A-FIG. 7C illustrates UBP chromosomal integration. FIG. 7A shows construction strategy of the arsB::UBP integration cassette. The integration cassette was constructed through overlapping PCR of a short UBP containing DNA and the neo cassette of the pKD13 plasmid. FIG. 7B shows successful integration of a chromosomal UBP was confirmed by PCR and biotin shift PCR. Confirmation of ΔrecA-Opt and Pol II+ΔrecA-Opt SSO integrant (A2 and B3 respectively) is shown. Teal bands indicate overexposure. FIG. 7C shows replating initial integrant and isolating individual clones quickly identified 100% retention clones for ΔrecA-Opt and Pol II+ΔrecA-Opt (A2.1 and B3.1 respectively). The same procedure for a WT-Opt integrant (C1) did not. A representative subset of replated clones is shown. Red bands indicate overexposure. In panels B and C, the identities of the primer sets used to produce each gel are given above each gel. Molecular weights are provided next to size standards in number of base pairs. When relevant % Shift values are provided below lanes and streptavidin-DNA and DNA species are indicated with black and red arrows, respectively.

FIG. 8A-FIG. 8B show reprogramed strains and chromosomal integrant doubling time characterization. FIG. 8A shows growth curves for reprogramed strains without a chromosomal UBP (WT-Opt (red), ΔrecA-Opt (blue), and Pol II+ΔrecA-Opt (gold)) and wild-type BL21(DE3) with chloramphenicol resistance (lacZYA::cat (black)) are shown. Circles/solid lines represent growth in media with dNaMTP and dTPT3TP. Squares/dotted lines represent growth in media without dNaMTP and dTPT3TP. FIG. 8B shows the average measured doubling times (n=3) are presented for all strains with and without a chromosomal UBP and with and without the addition of dXTPs. FIG. 6B discloses SEQ ID NOS 28 and 29, respectively, in order of appearance.

FIG. 9A-FIG. 9B shows Pol II+ΔrecA-Opt chromosomal UBP integrant contamination with WT-Opt cells. Replicate 3 of the Pol II+ΔrecA-Opt integrant was contaminated with WT-Opt cells at passage 13. FIG. 9A shows the PpolB locus was monitored by PCR of gDNA samples from passaging of replicate 3 for the Pol II+ΔrecA-Opt integrant. Strains with the PPol II+ mutation produce a larger amplicon than wild-type BL21(DE3) with chloramphenicol resistance (lacZYA::cat) (a) as seen from analysis of Pol II+ΔrecA-Opt before UBP integration (b). FIG. 9B shows the recA locus was monitored by PCR of gDNA samples from passaging of replicate 3 for the Pol II+ΔrecA-Opt integrant. Strains with the ΔrecA mutation produce a smaller amplicon than wild-type BL21(DE3) with chloramphenicol resistance (lacZYA::cat) (a) as seen from analysis of Pol II+ΔrecA-Opt before UBP integration (b).

FIG. 10A-FIG. 10B illustrates WT-Opt chromosomal UBP integrant PtNTT2(66-575) mutation during passaging. A depiction of PtNTT2(66-575) mutation during passaging of WT-Opt and its characterization. FIG. 10A shows during passaging WT-Opt mutants arose where region between cat and IS1 (top panel) was truncated to the C-terminus of PtNTT2(66-575) and IS1 (middle panel). Sequencing confirmed this transposition (bottom panel). FIG. 10A discloses SEQ ID NOS 30-32, respectively, in order of appearance. FIG. 10B shows inactivation of PtNTT2(66-575) by the IS1 transposon was monitored by PCR of gDNA from passaging of WT-Opt (See Table S1 for primers). Transposition events inactivate PtNTT2(66-575) and range in size between ˜3000-4000 bp. Inactivation occurs during the rapid phase of UBP loss. An additional amplicon (approximately 1500 bp in size) is also produced by these primers in wild-type BL21(DE3) with chloramphenicol resistance (lacZYA::cat) (a), WT-Opt before UBP integration (b), and wild-type BL21(DE3) (c).

### DETAILED DESCRIPTION OF THE INVENTION

The development of an unnatural base pair (UBP) allowing cells to store and retrieve increased information has a profound effect in practical applications, including human health applications by facilitating the production of proteins containing unnatural amino acids for development as therapeutics. However, retention of the UBP within a population of cells is sequence-dependent and in some sequences, the UBP is not sufficiently maintained or maintained at a reduced level, for practical applications (e.g. protein expression), thereby limiting the number of codons available for use.

Although loss of the UBP during extended growth may be mitigated by applying selection pressure for triphosphate uptake and UBP retention via expression of Cas9 directed to cleave, and thus degrade DNA sequences that have lost the UBP, retention remains challenging in some sequence contexts. Moreover, this approach requires optimizing different guide RNAs for every sequence to be retained, which is challenging with many applications, for example, those involving propagation of random DNA sequences. In addition, encoding information with the UBP in the chromosome as opposed to a plasmid, was expected to be incompatible with applying this selection pressure due to undesired cleavage of UBP-containing sequences and/or because cleavage would result in destruction of the chromosome as opposed to the less consequential elimination of one of many copies of a plasmid.

Disclosed herein, in some embodiments are methods, compositions, cells, engineered microorganisms, plasmids, and kits for increased retention of a UBP utilizing modified DNA repair-associated proteins, e.g., proteins involved in recombinational repair, SOS response, nucleotide excision repair, or methyl-directed mismatch repair, and/or modified transposition-associated proteins, e.g., Insertion element IS1 4 protein InsB, Insertion element IS1 4 protein InsA. In some instances, constitutive expression or overexpression of DNA repair-associated proteins, and/or deletion of, or reduced expression, of transposition-associated proteins, facilitates the increased stability of the nucleoside triphosphate transporter resulting in the creation of SSOs characterized by an increased in UBP chromosomal retention.

Disclosed herein, in certain embodiments, are methods, compositions, cells, engineered microorganisms, plasmids, and kits for increased production of a nucleic acid molecule that comprises an unnatural nucleotide. In some instances, disclosed herein are engineered cells comprising: (a) a first nucleic acid molecule comprising an unnatural nucleotide; and (b) a second nucleic acid molecule encoding a modified transposition-associated protein. In some embodiments, the engineered cell further comprises a third nucleic acid molecule encoding a modified nucleoside triphosphate transporter, wherein the third nucleic acid molecule is incorporated in a genomic sequence of the engineer host cell, or comprises a plasmid encoding the modified nucleoside triphosphate transporter. In some embodiments, the engineered cell further comprises a Cas9 polypeptide or variants thereof, and a single guide RNA (sgRNA) comprising a crRNA-tracrRNA scaffold, wherein the combination of Cas9 polypeptide or variants thereof and sgRNA modulates replication of the first nucleic acid molecule encoding the unnatural nucleotide. In certain embodiments, the engineered cell further comprises: (a) a fourth nucleic acid molecule encoding a Cas9 polypeptide or variants thereof, and (b) a fifth nucleic acid molecule encoding a single guide RNA (sgRNA) comprising a crRNA-tracrRNA scaffold. In some instances, the first, second, third, fourth, and fifth nucleic acid molecules, are encoded in one or more plasmids, and the sgRNA encoded by the fifth nucleic acid molecule comprises a target motif that recognizes a modification at the unnatural nucleotide position within the first nucleic acid molecule.

In some embodiments, further provided herein include a nucleic acid molecule containing an unnatural nucleotide produced by a process comprising incubating an engineered cell with: (a) a first nucleic acid molecule comprising an unnatural nucleotide; (b) a second nucleic acid molecule encoding a modified transposition-associated protein; (c) a third nucleic acid molecule encoding a modified nucleoside triphosphate transporter; (d) a fourth nucleic acid molecule encoding a Cas9 polypeptide or variants thereof, and (e) a fifth nucleic acid molecule encoding a single guide RNA (sgRNA) comprising a crRNA-tracrRNA scaffold. In some instances, a modification at the unnatural nucleotide position within the first nucleic acid molecule generates a modified first nucleic acid molecule, and the combination of the Cas9 polypeptide or variants thereof and sgRNA modulates replication of the modified first nucleic acid molecule leading to production of the nucleic acid molecule containing an unnatural nucleotide. In some instances, expression of the modified transposition-associated protein in the engineered cell increases the stability of the triphosphate transporter. In some embodiments, increased stability of the triphosphate transporter contributes to an increase in the (i) production of a modified polypeptide comprising an unnatural amino acid encoded by the unnatural nucleotide, and/or (ii) increased retention of the unnatural nucleotide in the genome of the engineered cell.

In some embodiments, additional provided herein include a semi-synthetic organism (SSO) produced by a process comprising incubating an organism with: (a) a first nucleic acid molecule comprising an unnatural nucleotide; (b) a second nucleic acid molecule encoding a modified transposition-associated protein; (c) a third nucleic acid molecule encoding a modified nucleoside triphosphate transporter; (d) a fourth nucleic acid molecule encoding a Cas9 polypeptide or variants thereof, and (e) a fifth nucleic acid molecule encoding a single guide RNA (sgRNA) comprising a crRNA-tracrRNA scaffold. In some instances, a modification at the unnatural nucleotide position within the first nucleic acid molecule generates a modified first nucleic acid molecule, and the combination of the Cas9 polypeptide or variants thereof and the sgRNA modulates replication of the modified first nucleic acid molecule leading to production of the semi-synthetic organism containing a nucleic acid molecule comprising an unnatural nucleotide. In some instances, expression of the modified transposition-associated protein in the engineered cell increases the stability of the triphosphate transporter. In some embodiments, increased stability of the triphosphate transporter contributes to an increase in the (i) production of a modified polypeptide comprising an unnatural amino acid encoded by the unnatural nucleotide, and/or (ii) increased retention of the unnatural nucleotide in the genome of the SSO.

### DNA Repair Machinery

DNA repair mechanisms include nucleotide excision repair (NER), ribonucleotide excision repair (RER), the SOS response, methyl-directed mismatch repair (MMR), and recombinational repair. NER, MMR, RER, and the SOS response, are induced by signals, which may be mimicked by UBP introduction into the host genome. Non-limiting examples of DNA repair-associated proteins in prokaryotic cells involved in the recombinational repair and/or SOS response include RecA, Rad51, RadA, and LexA. Non-limiting examples of DNA repair-associated proteins in prokaryotic cells involved in recombinational repair include RecO, RecR, RecN, and RuvABC. Non-limiting examples of DNA repair-associated proteins in prokaryotic cells involved in NER include UvrA, and UvrB. Non-limiting examples of DNA repair-associated proteins in prokaryotic cells involved in MMR include MutS, MutH, and MutL.

In some embodiments, modified DNA repair-associated proteins are introduced in an engineered cell, or SSO, described herein, to increase chromosomal UBP retention. In some embodiments, the modified DNA repair-associated proteins comprise a deletion of RecA, Rad51, RadA, LexA, RecO, RecR, RecN, RuvABC, MutS, MutH, MutL, UvrA, and/or UvrB. In some embodiments, the deletion comprises an N-terminal deletion, a C-terminal deletion, a truncation at both termini, an internal deletion, and/or a deletion of the entire gene. In some embodiments, a deletion or mutation in a nucleic acid molecule encoding the DNA repair-associated protein is modified to achieve the deletion.

### Transposition-Associated Proteins

In E. coli, there are replicative and conservative (nonreplicative) modes of transposition of a transposable element (e.g., ISI) comprises of a nucleic acid sequence. In the replicative pathway, a new copy of the transposable element is generated in the transposition event. The results of the transposition are that one copy appears at the new site and one copy remains at the old site. In the conservative pathway, there is no replication. Instead, the element is excised from the chromosome or plasmid and is integrated into the new site. In these cases, DNA replication of the element does not occur, and the element is lost in the site of the original chromosome. Deletions of transposable elements cause a high incidence of deletions in their vicinity (e.g., deletion of the transposable element in addition to flanking or surrounding DNA).

The insB-4 and insA-4 genes encode two proteins required for the transposition of the IS1 transposon, InsB, and InsA. The IS1 transposition results in a 9 to 8 base pair target duplication. Deletion of insB-4 results in repression of aberrant transposition events mediated by InsB.

In some embodiments, the methods, engineered cells, and semi-synthetic organisms, described herein comprise a modified nucleic acid molecule encoding a transposition-associated protein. In some embodiments, the transposition-associated protein comprises insB, and/or insA. In some embodiments, the modified nucleic acid molecule encoding a transposition-associated protein comprises a deletion, or a mutation. In some embodiments, the deletion comprises an N-terminal deletion, a C-terminal deletion, a truncation at both termini, an internal deletion, and/or a deletion of the entire gene. In some embodiments, the mutation results in decreased expression of insB and/or InsA. In some embodiments, the deletion or mutation of the modified nucleic acid molecule encoding the transposition-associated protein is effective to stabilize expression and/or activity of the triphosphate nucleotide transporter, thereby increasing retention of the UBP.

In some embodiments, the methods, engineered cells, and semi-synthetic organisms, described herein comprise a modified nucleic acid molecule encoding the IS1 transposable element. In some embodiments, the modified nucleic acid molecule encoding the IS1 transposable element comprises a deletion, or a mutation. In some embodiments, the deletion comprises a knockout or knockdown of all, or part of the nucleic acid molecule encoding the IS1 transposon. In some embodiments, the mutation results in decreased expression of the IS1 transposon. In some embodiments, the deletion or mutation of the modified nucleic acid molecule encoding the IS1 transposon is effective to stabilize expression and/or activity of the triphosphate nucleotide transporter, thereby increasing retention of the UBP. In some instances, the modified nucleic acid molecule encoding the IS1 transposable element comprises SEQ ID NO. 4.

### CRISPR/CRISPR-associated (Cas) Editing System

In some embodiments, methods, cells, and engineered microorganisms disclosed herein utilize a CRISPR/CRISPR-associated (Cas) system for modification of a nucleic acid molecule comprising an unnatural nucleotide. In some instances, the CRISPR/Cas system modulates retention of a modified nucleic acid molecule that comprises a modification at its unnatural nucleotide position. In some instances, the retention is a decrease in replication of the modified nucleic acid molecule. In some instances, the CRISPR/Cas system generates a double-stranded break within a modified nucleic acid molecule leading to degradation involving DNA repair proteins such as RecBCD and its associated nucleases.

In some embodiments, the CRISPR/Cas system involves (1) an integration of short regions of genetic material that are homologous to a nucleic acid molecule of interest comprising an unnatural nucleotide, called “spacers”, in clustered arrays in the host genome, (2) expression of short guiding RNAs (crRNAs) from the spacers, (3) binding of the crRNAs to specific portions of the nucleic acid molecule of interest referred to as protospacers, and (4) degradation of protospacers by CRISPR-associated nucleases (Cas). In some cases, a Type-II CRISPR system has been described in the bacterium Streptococcus pyogenes, in which Cas9 and two non-coding small RNAs (pre-crRNA and tracrRNA (trans-activating CRISPR RNA)) act in concert to target and degrade a nucleic acid molecule of interest in a sequence-specific manner (Jinek et al., “A Programmable Dual-RNA-Guided DNA Endonuclease in Adaptive Bacterial Immunity,” Science 337(6096):816-821 (August 2012, epub Jun. 28, 2012)).

In some instances, the two noncoding RNAs are further fused into one single guide RNA (sgRNA). In some instances, the sgRNA comprises a target motif that recognizes a modification at the unnatural nucleotide position within a nucleic acid molecule of interest. In some embodiments, the modification is a substitution, insertion, or deletion. In some cases, the sgRNA comprises a target motif that recognizes a substitution at the unnatural nucleotide position within a nucleic acid molecule of interest. In some cases, the sgRNA comprises a target motif that recognizes a deletion at the unnatural nucleotide position within a nucleic acid molecule of interest. In some cases, the sgRNA comprises a target motif that recognizes an insertion at the unnatural nucleotide position within a nucleic acid molecule of interest.

In some cases, the target motif is between 10 to 30 nucleotides in length. In some instances, the target motif is between 15 to 30 nucleotides in length. In some cases, the target motif is about 11, 12, 13, 14, 15, 16, 17, 18, 19, 20, 21, 22, 23, 24, 25, 26, 27, 28, 29, or 30 nucleotides in length. In some cases, the target motif is about 15, 16, 17, 18, 19, 20, 21, or 22 nucleotides in length.

In some cases, the sgRNA further comprises a protospacer adjacent motif (PAM) recognition element. In some instances, PAM is located adjacent to the 3′ terminus of the target motif. In some cases, a nucleotide within the target motif that forms Watson-Crick base pairing with the modification at the unnatural nucleotide position within the nucleic acid molecule of interest is located between 3 to 22, between 5 to 20, between 5 to 18, between 5 to 15, between 5 to 12, or between 5 to 10 nucleotides from the 5′ terminus of PAM. In some cases, a nucleotide within the target motif that forms Watson-Crick base pairing with the modification at the unnatural nucleotide position within the nucleic acid molecule of interest is located about 3, 4, 5, 6, 7, 8, 9, 10, 11, 12, 13, 14, or 15 nucleotides from the 5′ terminus of PAM.

In some instances, a CRISPR/Cas system utilizes a Cas9 polypeptide or a variant thereof. Cas9 is a double stranded nuclease with two active cutting sites, one for each strand of the double helix. In some instances, the Cas9 polypeptide or variants thereof generate a double-stranded break. In some cases, the Cas9 polypeptide is a wild-type Cas9. In some instances, the Cas9 polypeptide is an optimized Cas9 for expression in a cell and/or engineered microorganism described herein.

In some embodiments, the Cas9/sgRNA complex binds to a portion of the nucleic acid molecule of interest (e.g., DNA) that contains a sequence match to, for example, the 17-20 nucleotides of the sgRNA upstream of PAM. Once bound, two independent nuclease domains in Cas9 then each cleaves one of the DNA strands 3 bases upstream of the PAM, leaving a blunt end DNA double stranded break (DSB). The presence of DSB then results, in some instances, to degradation of the DNA of interest by RecBCD and its associated nucleases.

In some instances, the Cas9/sgRNA complex modulates retention of a modified nucleic acid molecule that comprises a modification at its unnatural nucleotide position. In some instances, the retention is a decrease in replication of the modified nucleic acid molecule. In some cases, the Cas9/sgRNA decreases the replication rate of the modified nucleic acid molecule by about 80%, 85%, 95%, 99%, or higher.

In some instances, the production of the nucleic acid molecule comprising an unnatural nucleotide increases by about 30%, 40%, 50%, 55%, 60%, 65%, 70%, 75%, 80%, 85%, 90%, 95%, 96%, 97%, 98%, 99%, or higher. In some instances, the production of the nucleic acid molecule comprising an unnatural nucleotide increases by about 50%, 60%, 70%, 80%, 90%, 95%, 96%, 97%, 98%, 99%, or higher.

In some cases, the retention of the nucleic acid molecule comprising an unnatural nucleotide increases by about 30%, 40%, 50%, 55%, 60%, 65%, 70%, 75%, 80%, 85%, 90%, 95%, 96%, 97%, 98%, 99%, or higher. In some instances, the retention of the nucleic acid molecule comprising an unnatural nucleotide increases by about 50%, 60%, 70%, 80%, 90%, 95%, 96%, 97%, 98%, 99%, or higher.

In some embodiments, the CRISPR/Cas system comprises two or more sgRNAs. In some instances, each of the two or more sgRNAs independently comprises a target motif that recognizes a modification at the unnatural nucleotide position within a nucleic acid molecule of interest. In some embodiments, the modification is a substitution, insertion, or deletion. In some cases, each of the two or more sgRNAs comprises a target motif that recognizes a substitution at the unnatural nucleotide position within a nucleic acid molecule of interest. In some cases, each of the two or more sgRNAs comprises a target motif that recognizes a deletion at the unnatural nucleotide position within a nucleic acid molecule of interest. In some cases, each of the two or more sgRNAs comprises a target motif that recognizes an insertion at the unnatural nucleotide position within a nucleic acid molecule of interest.

In some embodiments, the specificity of binding of the CRISPR components to the nucleic acid molecule of interest is controlled by the non-repetitive spacer elements in the pre-crRNA portion of sgRNA, which upon transcription along with the tracrRNA portion, directs the Cas9 nuclease to the protospacer:crRNA heteroduplex and induces double-strand breakage (DSB) formation. In some instances, the specificity of sgRNA is about 80%, 85%, 90%, 95%, 96%, 97%, 98%, 99%, or higher. In some instances, sgRNA has less than about 20%, 15%, 10%, 5%, 3%, 1%, or less off-target binding rate.

### Nucleic Acid Molecules

In some embodiments, a nucleic acid (e.g., also referred to herein as nucleic acid molecule of interest) is from any source or composition, such as DNA, cDNA, gDNA (genomic DNA), RNA, siRNA (short inhibitory RNA), RNAi, tRNA, mRNA or rRNA (ribosomal RNA), for example, and is in any form (e.g., linear, circular, supercoiled, single-stranded, double-stranded, and the like). In some embodiments, nucleic acids comprise nucleotides, nucleosides, or polynucleotides. In some cases, nucleic acids comprise natural and unnatural nucleic acids. In some cases, a nucleic acid also comprises unnatural nucleic acids, such as DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. For RNA, the uracil base is uridine. A nucleic acid sometimes is a vector, plasmid, phagemid, autonomously replicating sequence (ARS), centromere, artificial chromosome, yeast artificial chromosome (e.g., YAC) or other nucleic acid able to replicate or be replicated in a host cell. In some cases, an unnatural nucleic acid is a nucleic acid analogue. In additional cases, an unnatural nucleic acid is from an extracellular source. In other cases, an unnatural nucleic acid is available to the intracellular space of an organism provided herein, e.g., a genetically modified organism.

### Unnatural Nucleic Acids

A nucleotide analog, or unnatural nucleotide, comprises a nucleotide which contains some type of modification to either the base, sugar, or phosphate moieties. In some embodiments, a modification comprises a chemical modification. In some cases, modifications occur at the 3′OH or 5′OH group, at the backbone, at the sugar component, or at the nucleotide base. Modifications, in some instances, optionally include non-naturally occurring linker molecules and/or of interstrand or intrastrand cross links. In one aspect, the modified nucleic acid comprises modification of one or more of the 3′OH or 5′OH group, the backbone, the sugar component, or the nucleotide base, and/or addition of non-naturally occurring linker molecules. In one aspect, a modified backbone comprises a backbone other than a phosphodiester backbone. In one aspect, a modified sugar comprises a sugar other than deoxyribose (in modified DNA) or other than ribose (modified RNA). In one aspect, a modified base comprises a base other than adenine, guanine, cytosine or thymine (in modified DNA) or a base other than adenine, guanine, cytosine or uracil (in modified RNA).

In some embodiments, the nucleic acid comprises at least one modified base. In some instances, the nucleic acid comprises 2, 3, 4, 5, 6, 7, 8, 9, 10, 15, 20, or more modified bases. In some cases, modifications to the base moiety include natural and synthetic modifications of A, C, G, and T/U as well as different purine or pyrimidine bases. In some embodiments, a modification is to a modified form of adenine, guanine cytosine or thymine (in modified DNA) or a modified form of adenine, guanine cytosine or uracil (modified RNA).

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CI 1/4) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N₄-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crookeand Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993.

Unnatural nucleic acids comprising various heterocyclic bases and various sugar moieties (and sugar analogs) are available in the art, and the nucleic acid in some cases include one or several heterocyclic bases other than the principal five base components of naturally-occurring nucleic acids. For example, the heterocyclic base includes, in some cases, uracil-5-yl, cytosine-5-yl, adenin-7-yl, adenin-8-yl, guanin-7-yl, guanin-8-yl, 4-aminopyrrolo [2.3-d]pyrimidin-5-yl, 2-amino-4-oxopyrolo [2, 3-d]pyrimidin-5-yl, 2-amino-4-oxopyrrolo [2.3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, a modified base of a unnatural nucleic acid is depicted below, wherein the wavy line identifies a point of attachment to the (deoxy)ribose or ribose.

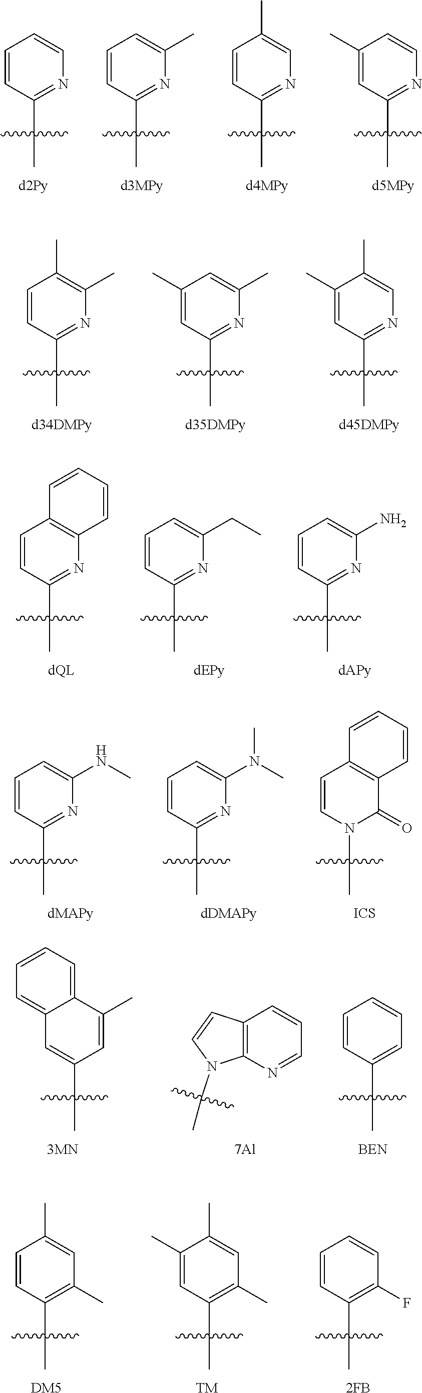

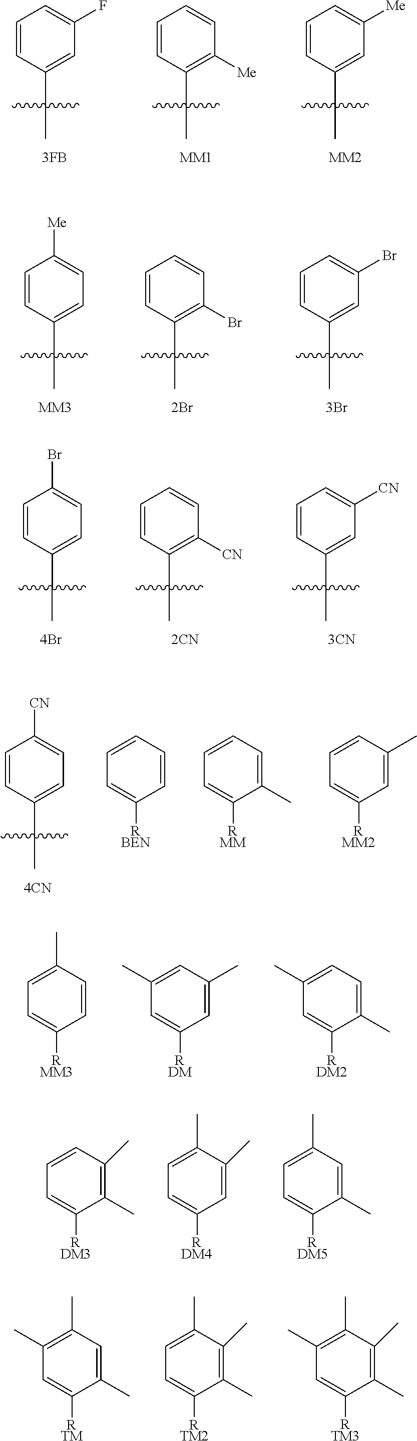

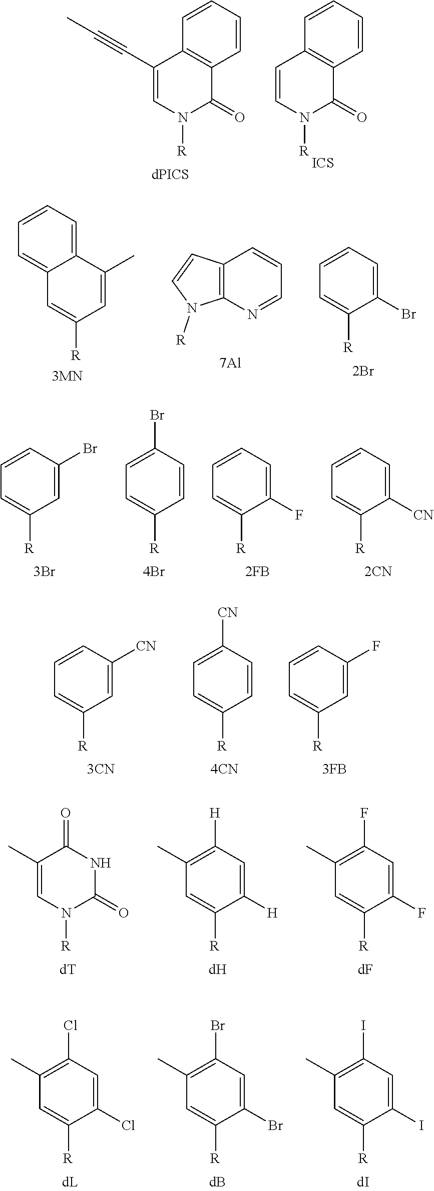

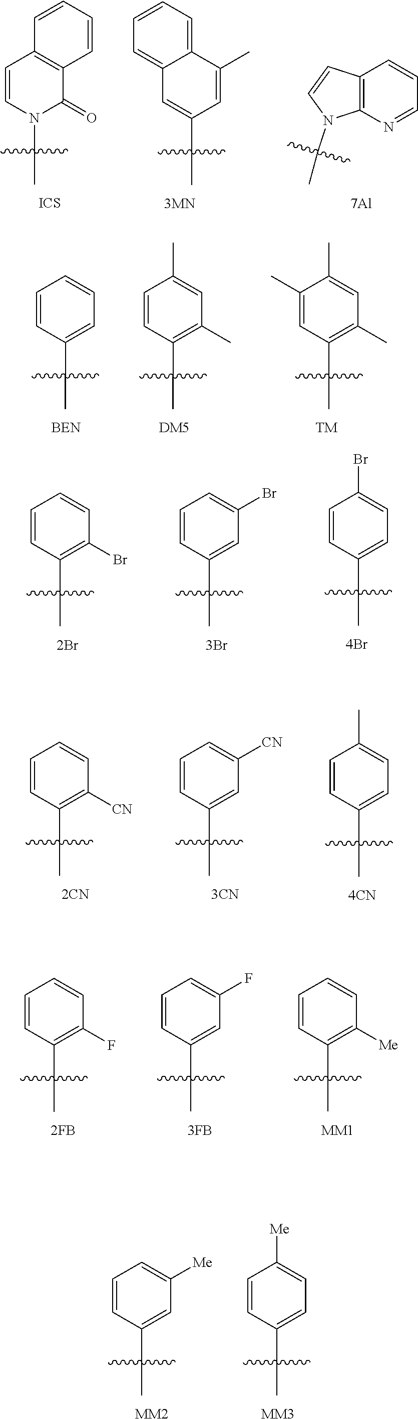

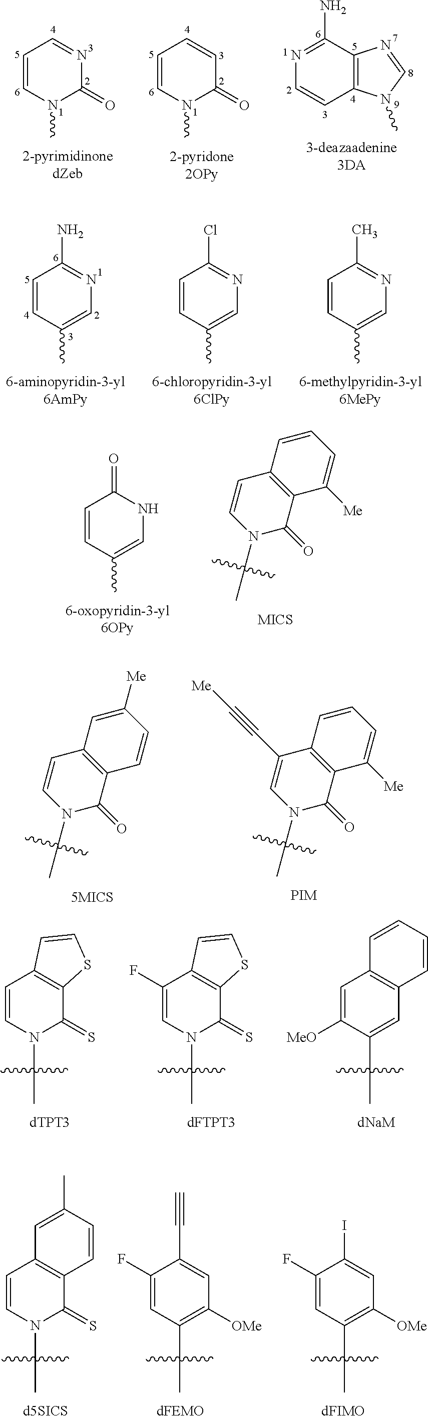

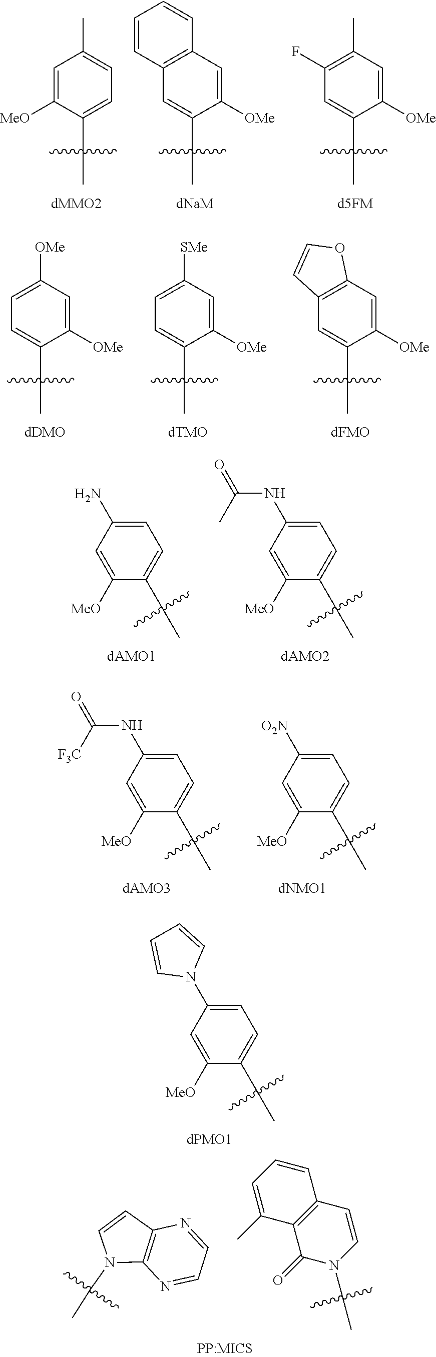

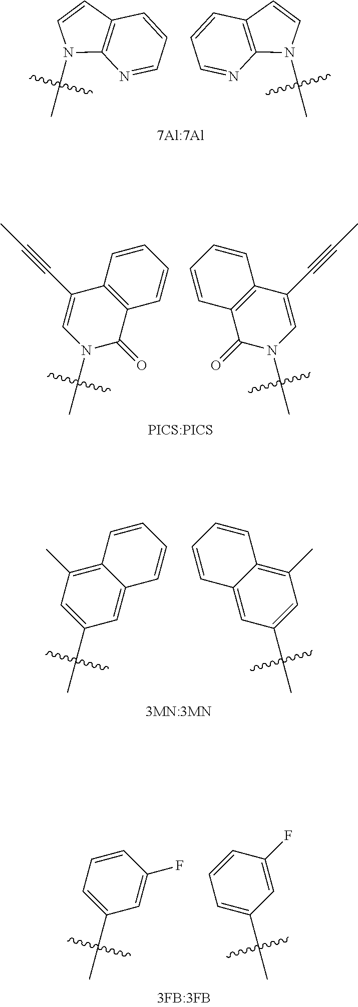

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotri ester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337).

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substitutent groups (including 5′ and/or 2′ substituent groups; bridging of two ring atoms to form bicyclic nucleic acids (BNA); replacement of the ribosyl ring oxygen atom with S, N(R), or C(Ri)(R₂) (R═H, C₁-C₁₂ alkyl or a protecting group); and combinations thereof. Examples of chemically modified sugars can be found in WO2008/101157, US2005/0130923, and WO2007/134181.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)n CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM50J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Nucleic Acid Base Pairing Properties

In some embodiments, an unnatural nucleic acid forms a base pair with another nucleic acid. In some embodiments, a stably integrated unnatural nucleic acid is an unnatural nucleic acid that can form a base pair with another nucleic acid, e.g., a natural or unnatural nucleic acid. In some embodiments, a stably integrated unnatural nucleic acid is an unnatural nucleic acid that can form a base pair with another unnatural nucleic acid (unnatural nucleic acid base pair (UBP)). For example, a first unnatural nucleic acid can form a base pair with a second unnatural nucleic acid. For example, one pair of unnatural nucleotide triphosphates that can base pair when incorporated into nucleic acids include a triphosphate of d5SICS (d5SICSTP) and a triphosphate of dNaM (dNaMTP). Such unnatural nucleotides can have a ribose or deoxyribose sugar moiety. In some embodiments, an unnatural nucleic acid does not substantially form a base pair with a natural nucleic acid (A, T, G, C). In some embodiments, a stably integrated unnatural nucleic acid can form a base pair with a natural nucleic acid.

In some embodiments, a stably integrated unnatural nucleic acid is an unnatural nucleic acid that can form a UBP, but does not substantially form a base pair with each of the four natural nucleic acids. In some embodiments, a stably integrated unnatural nucleic acid is an unnatural nucleic acid that can form a UBP, but does not substantially form a base pair with one or more natural nucleic acids. For example, a stably integrated unnatural nucleic acid may not substantially form a base pair with A, T, and, C, but can form a base pair with G. For example, a stably integrated unnatural nucleic acid may not substantially form a base pair with A, T, and, G, but can form a base pair with C. For example, a stably integrated unnatural nucleic acid may not substantially form a base pair with C, G, and, A, but can form a base pair with T. For example, a stably integrated unnatural nucleic acid may not substantially form a base pair with C, G, and, T, but can form a base pair with A. For example, a stably integrated unnatural nucleic acid may not substantially form a base pair with A and T, but can form a base pair with C and G. For example, a stably integrated unnatural nucleic acid may not substantially form a base pair with A and C, but can form a base pair with T and G. For example, a stably integrated unnatural nucleic acid may not substantially form a base pair with A and G, but can form a base pair with C and T. For example, a stably integrated unnatural nucleic acid may not substantially form a base pair with C and T, but can form a base pair with A and G. For example, a stably integrated unnatural nucleic acid may not substantially form a base pair with C and G, but can form a base pair with T and G. For example, a stably integrated unnatural nucleic acid may not substantially form a base pair with T and G, but can form a base pair with A and G. For example, a stably integrated unnatural nucleic acid may not substantially form a base pair with, G, but can form a base pair with A, T, and, C. For example, a stably integrated unnatural nucleic acid may not substantially form a base pair with, A, but can form a base pair with G, T, and, C. For example, a stably integrated unnatural nucleic acid may not substantially form a base pair with, T, but can form a base pair with G, A, and, C. For example, a stably integrated unnatural nucleic acid may not substantially form a base pair with, C, but can form a base pair with G, T, and, A.

Exemplary, unnatural nucleotides capable of forming an unnatural DNA or RNA base pair (UBP) under conditions in vivo includes, but is not limited to, 5SICS, d5SICS, NAM, dNaM, dTPT3, and combinations thereof. In some embodiments, unnatural nucleotides include:

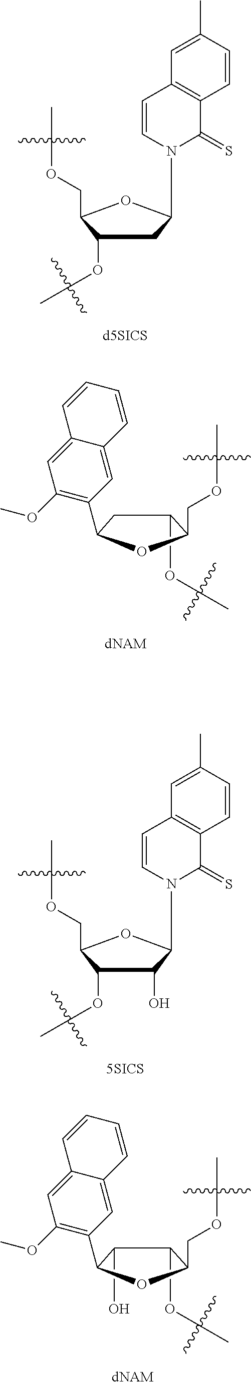

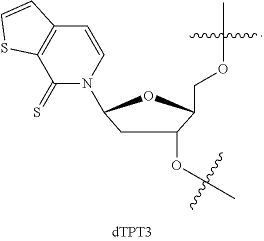

### Engineered Organism

In some embodiments, methods and plasmids disclosed herein is further used to generate engineered organism, e.g. an organism that incorporates and replicates an unnatural nucleotide or an unnatural nucleic acid base pair (UBP) with improved UBP retention and also transcribes and translates the nucleic acid containing the unnatural nucleotide or unnatural nucleic acid base pair into a protein containing an unnatural amino acid residue. In some instances, the organism is a semi-synthetic organism (SSO). In some instances, the SSO is a cell.

In some instances, the cell employed is genetically transformed with an expression cassette encoding a heterologous protein, e.g., a nucleoside triphosphate transporter capable of transporting unnatural nucleotide triphosphates into the cell, a modified transposition-associated protein to increase stability of the nucleotide triphosphate transporter, a CRISPR/Cas9 system to remove modifications at the unnatural nucleotide triphosphate positions, and/or a polymerase with high fidelity for an unnatural nucleic acid, so that the unnatural nucleotides are incorporated into cellular nucleic acids and e.g., form unnatural base pairs under in vivo conditions. In some instances, cells further comprise enhanced activity for unnatural nucleic acid uptake. In some cases, cells further comprise enhanced activity for unnatural nucleic acid import. In some cases, cells further comprise enhanced polymerase activity for unnatural nucleic acids.

In some embodiments, Cas9 and sgRNA are encoded on separate plasmids. In some instances, Cas9 and sgRNA are encoded on the same plasmid. In some cases, the nucleic acid molecule encoding Cas9, sgRNA, or a nucleic acid molecule comprising an unnatural nucleotide are located on one or more plasmids. In some instances, Cas9 is encoded on a first plasmid and the sgRNA and the nucleic acid molecule comprising an unnatural nucleotide are encoded on a second plasmid. In some instances, Cas9, sgRNA, and the nucleic acid molecule comprising an unnatural nucleotide are encoded on the same plasmid. In some instances, the nucleic acid molecule comprises two or more unnatural nucleotides.

In some instances, a first plasmid encoding Cas9 and sgRNA and a second plasmid encoding a nucleic acid molecule comprising an unnatural nucleotide are introduced into an engineered microorganism. In some instances, a first plasmid encoding Cas9 and a second plasmid encoding sgRNA and a nucleic acid molecule comprising an unnatural nucleotide are introduced into an engineered microorganism. In some instances, a plasmid encoding Cas9, sgRNA and a nucleic acid molecule comprising an unnatural nucleotide is introduced into an engineered microorganism. In some instances, the nucleic acid molecule comprises two or more unnatural nucleotides.

In some embodiments, a living cell is generated that incorporates within its nucleic acids at least one unnatural nucleotide and/or at least one unnatural base pair (UBP). In some instances, the unnatural base pair includes a pair of unnatural mutually base-pairing nucleotides capable of forming the unnatural base pair under in vivo conditions, when the unnatural mutually base-pairing nucleotides, as their respective triphosphates, are taken up into the cell by action of a nucleotide triphosphate transporter. The cell can be genetically transformed by an expression cassette encoding a nucleotide triphosphate transporter so that the nucleotide triphosphate transporter is expressed and is available to transport the unnatural nucleotides into the cell. The cell can be genetically transformed by an expression cassette encoding a polymerase so that the polymerase is expressed and is available to incorporate unnatural nucleotides into the cell's nucleic acids. The cell can be a prokaryotic or eukaryotic cell, and the pair of unnatural mutually base-pairing nucleotides, as their respective triphosphates, can be a triphosphate of d5SICS (d5SICSTP) and a triphosphate of dNaM (dNaMTP).

In some embodiments, cells are genetically transformed cells with a nucleic acid, e.g., an expression cassette encoding a nucleotide triphosphate transporter capable of transporting such unnatural nucleotides into the cell. A cell can comprise a heterologous nucleotide triphosphate transporter, where the heterologous nucleotide triphosphate transporter can transport natural and unnatural nucleotide triphosphates into the cell. A cell can comprise a heterologous polymerase, where the heterologous polymerase has activity for an unnatural nucleic acid.

In some cases, a method described herein also include contacting a genetically transformed cell with the respective triphosphate forms unnatural nucleotides, in the presence of potassium phosphate and/or an inhibitor of phosphatases or nucleotidases. During or after such contact, the cell can be placed within a life-supporting medium suitable for growth and replication of the cell. The cell can be maintained in the life-supporting medium so that the respective triphosphate forms of unnatural nucleotides are incorporated into nucleic acids within the cells, and through at least one replication cycle of the cell. The pair of unnatural mutually base-pairing nucleotides as a respective triphosphate, can comprise a triphosphate of d5SICS (d5SICSTP) and a triphosphate of dNaM (dNaMTP), the cell can be E. coli, and the d5SICSTP and dNaMTP can be efficiently imported into E. coli by the transporter PtNTT2, wherein an E. coli polymerase, such as Pol I, can efficiently use the unnatural triphosphates to replicate DNA, thereby incorporating unnatural nucleotides and/or unnatural base pairs into cellular nucleic acids within the cellular environment.

By practice of a method of the invention, the person of ordinary skill can obtain a population of a living and propagating cells that has at least one unnatural nucleotide and/or at least one unnatural base pair (UBP) within at least one nucleic acid maintained within at least some of the individual cells, wherein the at least one nucleic acid is stably propagated within the cell, and wherein the cell expresses a nucleotide triphosphate transporter suitable for providing cellular uptake of triphosphate forms of one or more unnatural nucleotides when contacted with (e.g., grown in the presence of) the unnatural nucleotide(s) in a life-supporting medium suitable for growth and replication of the organism.

After transport into the cell by the nucleotide triphosphate transporter, the unnatural base-pairing nucleotides are incorporated into nucleic acids within the cell by cellular machinery, e.g., the cell's own DNA and/or RNA polymerases, a heterologous polymerase, or a polymerase that has been evolved using directed evolution (Chen T, Romesberg FE, FEBS Lett. 2014 Jan. 21; 588(2):219-29; Betz K et al., J Am Chem Soc. 2013 Dec. 11; 135(49):18637-43). The unnatural nucleotides can be incorporated into cellular nucleic acids such as genomic DNA, genomic RNA, mRNA, structural RNA, microRNA, and autonomously replicating nucleic acids (e.g., plasmids, viruses, or vectors).

In some cases, genetically engineered cells are generated by introduction of nucleic acids, e.g., heterologous nucleic acids, into cells. Any cell described herein can be a host cell and can comprise an expression vector. In one embodiment, the host cell is a prokaryotic cell. In another embodiment, the host cell is E. coli. In some embodiments, a cell comprises one or more heterologous polynucleotides. Nucleic acid reagents can be introduced into microorganisms using various techniques. Non-limiting examples of methods used to introduce heterologous nucleic acids into various organisms include; transformation, transfection, transduction, electroporation, ultrasound-mediated transformation, particle bombardment and the like. In some instances the addition of carrier molecules (e.g., bis-benzimdazolyl compounds, for example, see U.S. Pat. No. 5,595,899) can increase the uptake of DNA in cells typically though to be difficult to transform by conventional methods. Conventional methods of transformation are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

In some instances, genetic transformation is obtained using direct transfer of an expression cassette, in but not limited to, plasmids, viral vectors, viral nucleic acids, phage nucleic acids, phages, cosmids, and artificial chromosomes, or via transfer of genetic material in cells or carriers such as cationic liposomes. Such methods are available in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, a nucleotide triphosphate transporter or polymerase nucleic acid molecule, expression cassette and/or vector can be introduced to a cell by any method including, but not limited to, calcium-mediated transformation, electroporation, microinjection, lipofection, particle bombardment and the like.

In some cases, a cell comprises unnatural nucleotide triphosphates incorporated into one or more nucleic acids within the cell. For example, the cell can be a living cell capable of incorporating at least one unnatural nucleotide within DNA or RNA maintained within the cell. The cell can also incorporate at least one unnatural base pair (UBP) comprising a pair of unnatural mutually base-pairing nucleotides into nucleic acids within the cell under in vivo conditions, wherein the unnatural mutually base-pairing nucleotides, e.g., their respective triphosphates, are taken up into the cell by action of a nucleotide triphosphate transporter, the gene for which is present (e.g., was introduced) into the cell by genetic transformation. For example, upon incorporation into the nucleic acid maintained within s cell, d5SICS and dNaM can form a stable unnatural base pair that can be stably propagated by the DNA replication machinery of an organism, e.g., when grown in a life-supporting medium comprising d5SICS and dNaM.

In some cases, cells are capable of replicating an unnatural nucleic acid. Such methods can include genetically transforming the cell with an expression cassette encoding a nucleotide triphosphate transporter capable of transporting into the cell, as a respective triphosphate, one or more unnatural nucleotides under in vivo conditions. Alternatively, a cell can be employed that has previously been genetically transformed with an expression cassette that can express an encoded nucleotide triphosphate transporter. The method can also include contacting or exposing the genetically transformed cell to potassium phosphate and the respective triphosphate forms of at least one unnatural nucleotide (for example, two mutually base-pairing nucleotides capable of forming the unnatural base pair (UBP)) in a life-supporting medium suitable for growth and replication of the cell, and maintaining the transformed cell in the life-supporting medium in the presence of the respective triphosphate forms of at least one unnatural nucleotide (for example, two mutually base-pairing nucleotides capable of forming the unnatural base pair (UBP)) under in vivo conditions, through at least one replication cycle of the cell.

In some embodiments, a cell comprises a stably incorporated unnatural nucleic acid. Some embodiments comprise a cell (e.g., as E. coli) that stably incorporates nucleotides other than A, G, T, and C within nucleic acids maintained within the cell. For example, the nucleotides other than A, G, T, and C can be d5SICS, dNaM, and dTPT3, which upon incorporation into nucleic acids of the cell, can form a stable unnatural base pair within the nucleic acids. In one aspect, unnatural nucleotides and unnatural base pairs can be stably propagated by the replication apparatus of the organism, when an organism transformed with the gene for the triphosphate transporter, is grown in a life-supporting medium that includes potassium phosphate and the triphosphate forms of d5SICS, dNaM, and dTPT3.

In some cases, a cell comprises an expanded genetic alphabet. A cell can comprise a stably incorporated unnatural nucleic acid. In some embodiments, a cell with an expanded genetic alphabet comprises an unnatural nucleic acid that can form a base pair (bp) with another nucleic acid, e.g., a natural or unnatural nucleic acid. In some embodiments, a cell with an expanded genetic alphabet comprises an unnatural nucleic acid that is hydrogen bonded to another nucleic acid. In some embodiments, a cell with an expanded genetic alphabet comprises an unnatural nucleic acid that is not hydrogen bonded to another nucleic acid to which it is base paired. In some embodiments, a cell with an expanded genetic alphabet comprises an unnatural nucleic acid that base pairs to another nucleic acid via hydrophobic interactions. In some embodiments, a cell with an expanded genetic alphabet comprises an unnatural nucleic acid that base pairs to another nucleic acid via non-hydrogen bonding interactions. A cell with an expanded genetic alphabet can be a cell that can copy a homologous nucleic acid to form a nucleic acid comprising an unnatural nucleic acid. A cell with an expanded genetic alphabet can be a cell comprising an unnatural nucleic acid base paired with another unnatural nucleic acid (unnatural nucleic acid base pair (UBP)).

In some embodiments, cells form unnatural DNA base pairs (UBPs) from the imported unnatural nucleotides under in vivo conditions. In some embodiments potassium phosphate and/or inhibitors of phosphatase and/or nucleotidase activities can facilitate transport of unnatural nucleic acids. The methods include use of a cell that expresses a heterologous nucleotide triphosphate transporter. When such a cell is contacted with one or more nucleotide triphosphates, the nucleotide triphosphates are transported into the cell. The cell can be in the presence of potassium phosphate and/or inhibitors of phosphatase and nucleotidase. Unnatural nucleotide triphosphates can be incorporated into nucleic acids within the cell by the cell's natural machinery and, for example, can mutually base-pair to form unnatural base pairs within the nucleic acids of the cell.

In some embodiments, a UBP can be incorporated into a cell or population of cells when exposed to unnatural triphosphates. In some embodiments a UBP can be incorporated into a cell or population of cells when substantially consistently exposed to unnatural triphosphates. In some embodiments, replication of a UBP does not result in a substantially reduced growth rate. In some embodiments, replication expression of a heterologous protein, e.g., a nucleotide triphosphate transport does not result in a substantially reduced growth rate.

In some embodiments, induction of expression of a heterologous gene, e.g., an NTT, in a cell can result in slower cell growth and increased unnatural nucleic acid uptake compared to the growth and uptake of a cell without induction of expression of the heterologous gene. In some embodiments, induction of expression of a heterologous gene, e.g., an NTT, in a cell can result in increased cell growth and increased unnatural nucleic acid uptake compared to the growth and uptake of a cell without induction of expression of the heterologous gene.

In some embodiments, a UBP is incorporated during a log growth phase. In some embodiments, a UBP is incorporated during a non-log growth phase. In some embodiments, a UBP is incorporated during a substantially linear growth phase. In some embodiments a UBP is stably incorporated into a cell or population of cells after growth for a time period. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 50 or more duplications. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, or24 hours of growth. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days of growth. For example, a UBP can be stably incorporated into a cell or population of cells after growth for at least about 1, 2, 3, 4, 5, 6, 7, 8, 9, 10, 11, or 12 months of growth. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50 years of growth.

In some embodiments, a cell further utilizes a polymerase described herein to generate a mutant mRNA which contains a mutant codon that comprises one or more unnatural nucleic acid base. In some instances, a cell further utilizes a polymerase disclosed herein to generate a mutant tRNA which contains a mutant anticodon that comprises one or more unnatural nucleic acid base. In some instances, the mutant anticodon represents an unnatural amino acid. In some instances, the anticodon of the mutant tRNA pairs with the codon of the mutant mRNA during translation to synthesis a protein that contains an unnatural amino acid.

As used herein, an amino acid residue can refer to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” can refer to a molecule containing both an amino group and a carboxyl group bound to a carbon which is designated the α-carbon.

The term “β-amino acid” can refer to a molecule containing both an amino group and a carboxyl group in a R configuration.

“Naturally occurring amino acid” can refer to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | Side- | Side-chain | |
|---|---|---|---|---|---|
| | Letter | Letter | chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Gin | E | polar | negative | −3.5 |
| Glutamine | Gin | Q | polar | neutral | −3.5 |
| Glycine | Gly | G | nonpolar | neutral | −0.4 |
| Histidine | His | H | polar | positive (10%) | −3.2 |
| | | | | neutral (90%) | |
| Isoleucine | He | I | nonpolar | neutral | 4.5 |
| Leucine | Len | L | nonpolar | neutral | 3.8 |
| Ly sine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” can be glycine, alanine, proline, and analogs thereof “Large hydrophobic amino acids” can be valine, leucine, isoleucine, phenylalanine, methionine, tryptophan, and analogs thereof. “Polar amino acids” can be serine, threonine, asparagine, glutamine, cysteine, tyrosine, and analogs thereof “Charged amino acids” can be lysine, arginine, histidine, aspartate, glutamate, and analogs thereof.

An “amino acid analog” can be a molecule which is structurally similar to an amino acid and which can be substituted for an amino acid in the formation of a peptidomimetic macrocycle Amino acid analogs include, without limitation, j-amino acids and amino acids where the amino or carboxy group is substituted by a similarly reactive group (e.g., substitution of the primary amine with a secondary or tertiary amine, or substitution of the carboxy group with an ester).

A “non-natural amino acid” can be an amino acid which is not one of the twenty amino acids commonly found in peptides synthesized in nature, and known by the one letter abbreviations A, R, N, C, D, Q, E, G, H, I, L, K, M, F, P, S, T, W, Y and V.

Amino acid analogs can include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-β-amino-4-(1-naphthyl)-butyric acid; (R)-β-amino-4-(2,4-dichlorophenyl)butyric acid; (R)-β-amino-4-(2-chlorophenyl)-butyric acid; (R)-β-amino-4-(2-cyanophenyl)-butyric acid; (R)-β-amino-4-(2-fluorophenyl)-butyric acid; (R)-β-amino-4-(2-furyl)-butyric acid; (R)-β-amino-4-(2-methylphenyl)-butyric acid; (R)-β-amino-4-(2-naphthyl)-butyric acid; (R)-β-amino-4-(2-thienyl)-butyric acid; (R)-β-amino-4-(2-trifluoromethylphenyl)-butyric acid; (R)-β-amino-4-(3,4-dichlorophenyl)butyric acid; (R)-β-amino-4-(3,4-difluorophenyl)butyric acid; (R)-β-amino-4-(3-benzothienyl)-butyric acid; (R)-β-amino-4-(3-chlorophenyl)-butyric acid; (R)-β-amino-4-(3-cyanophenyl)-butyric acid; (R)-β-amino-4-(3-fluorophenyl)-butyric acid; (R)-β-amino-4-(3-methylphenyl)-butyric acid; (R)-β-amino-4-(3-pyridyl)-butyric acid; (R)-β-amino-4-(3-thienyl)-butyric acid; (R)-β-amino-4-(3-trifluoromethylphenyl)-butyric acid; (R)-β-amino-4-(4-bromophenyl)-butyric acid; (R)-β-amino-4-(4-chlorophenyl)-butyric acid; (R)-β-amino-4-(4-cyanophenyl)-butyric acid; (R)-β-amino-4-(4-fluorophenyl)-butyric acid; (R)-β-amino-4-(4-iodophenyl)-butyric acid; (R)-β-amino-4-(4-methylphenyl)-butyric acid; (R)-β-amino-4-(4-nitrophenyl)-butyric acid; (R)-β-amino-4-(4-pyridyl)-butyric acid; (R)-β-amino-4-(4-trifluoromethylphenyl)-butyric acid; (R)-β-amino-4-pentafluoro-phenylbutyric acid; (R)-3-amino-5-hexenoic acid; (R)-β-amino-5-hexynoic acid; (R)-β-amino-5-phenylpentanoic acid; (R)-β-amino-6-phenyl-5-hexenoic acid; (S)-1,2,3,4-tetrahydro-isoquinoline-3-acetic acid; (S)-3-amino-4-(1-naphthyl)-butyric acid; (S)-β-amino-4-(2,4-dichlorophenyl)butyric acid; (S)-3-amino-4-(2-chlorophenyl)-butyric acid; (S)-β-amino-4-(2-cyanophenyl)-butyric acid; (S)-3-amino-4-(2-fluorophenyl)-butyric acid; (S)-β-amino-4-(2-furyl)-butyric acid; (S)-β-amino-4-(2-methylphenyl)-butyric acid; (S)-β-amino-4-(2-naphthyl)-butyric acid; (S)-β-amino-4-(2-thienyl)-butyric acid; (S)-β-amino-4-(2-trifluoromethylphenyl)-butyric acid; (S)-β-amino-4-(3,4-dichlorophenyl)butyric acid; (S)-β-amino-4-(3,4-difluorophenyl)butyric acid; (S)-β-amino-4-(3-benzothienyl)-butyric acid; (S)-β-amino-4-(3-chlorophenyl)-butyric acid; (S)-β-amino-4-(3-cyanophenyl)-butyric acid; (S)-β-amino-4-(3-fluorophenyl)-butyric acid; (S)-β-amino-4-(3-methylphenyl)-butyric acid; (S)-β-amino-4-(3-pyridyl)-butyric acid; (S)-β-amino-4-(3-thienyl)-butyric acid; (S)-β-amino-4-(3-trifluoromethylphenyl)-butyric acid; (S)-β-amino-4-(4-bromophenyl)-butyric acid; (S)-β-amino-4-(4-chlorophenyl) butyric acid; (S)-β-amino-4-(4-cyanophenyl)-butyric acid; (S)-β-amino-4-(4-fluorophenyl) butyric acid; (S)-β-amino-4-(4-iodophenyl)-butyric acid; (S)-β-amino-4-(4-methylphenyl)-butyric acid; (S)-β-amino-4-(4-nitrophenyl)-butyric acid; (S)-β-amino-4-(4-pyridyl)-butyric acid; (S)-β-amino-4-(4-trifluoromethylphenyl)-butyric acid; (S)-β-amino-4-pentafluoro-phenylbutyric acid; (S)-3-amino-5-hexenoic acid; (S)-β-amino-5-hexynoic acid; (S)-β-amino-5-phenylpentanoic acid; (S)-β-amino-6-phenyl-5-hexenoic acid; 1,2,5,6-tetrahydropyridine-3-carboxylic acid; 1,2,5,6-tetrahydropyridine-4-carboxylic acid; β-amino-3-(2-chlorophenyl)-propionic acid; β-amino-3-(2-thienyl)-propionic acid; β-amino-3-(3-bromophenyl)-propionic acid; β-amino-3-(4-chlorophenyl)-propionic acid; β-amino-3-(4-methoxyphenyl)-propionic acid; β-amino-4,4,4-trifluoro-butyric acid; β-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-No-benzyloxycarbonyl-3-homolysine; No-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs can include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH·dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; β-amino-4,4,4-trifluoro-butyric acid; 3-fluoro-valine; 4,4,4-trifluoro-valine; 4,5-dehydro-L-leu-OH·dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH·dicyclohexylammonium salt; cyclopentyl-Gly-OH·dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine·dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs can include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃) OH; Nδ-benzyloxycarbonyl-L-ornithine; Nω-nitro-D-arginine; No-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)₂-OH (symmetrical); Lys(ivDde)-OH; Lys(Me)₂-OH·HCl; Lys(Me3)-OH chloride; No-nitro-D-arginine; and No-nitro-L-arginine.

Amino acid analogs can include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs can include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs can include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs can include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs can include analogs of serine and threonine. Examples of amino acid analogs of serine and threonine include, but are not limited to, β-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs can include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

Amino acid analogs can be racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

In some embodiments, an unnatural amino acid is an unnatural amino acid described in Liu C. C., Schultz, P. G. Annu. Rev. Biochem. 2010, 79, 413. In some embodiments, an unnatural amino acid comprises N6(2-azidoethoxy)-carbonyl-L-lysine.

### Cell Types

In some embodiments, many types of cells/microorganisms are used, e.g., for transforming or genetically engineering. In some embodiments, a cell is a prokaryotic or eukaryotic cell. In some cases, the cell is a microorganism such as a bacterial cell, fungal cell, yeast, or unicellular protozoan. In other cases, the cell is a eukaryotic cell, such as a cultured animal, plant, or human cell. In additional cases, the cell is present in an organism such as a plant or animal.

In some embodiments, an engineered microorganism is a single cell organism, often capable of dividing and proliferating. A microorganism can include one or more of the following features: aerobe, anaerobe, filamentous, non-filamentous, monoploid, dipoid, auxotrophic and/or non-auxotrophic. In certain embodiments, an engineered microorganism is a prokaryotic microorganism (e.g., bacterium), and in certain embodiments, an engineered microorganism is a non-prokaryotic microorganism. In some embodiments, an engineered microorganism is a eukaryotic microorganism (e.g., yeast, fungi, amoeba). In some embodiments, an engineered microorganism is a fungus. In some embodiments, an engineered organism is a yeast.

Any suitable yeast may be selected as a host microorganism, engineered microorganism, genetically modified organism or source for a heterologous or modified polynucleotide. Yeast include, but are not limited to, Yarrowia yeast (e.g., Y. lipolytica (formerly classified as Candida lipolytica)), Candida yeast (e.g., C. revkaufi, C. viswanathii, C. pulcherrima, C. tropicalis, C. utilis), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutaneum), Pichia yeast (e.g., P. pastoris) and Lipomyces yeast (e.g., L. starkeyii, L. lipoferus). In some embodiments, a suitable yeast is of the genus Arachniotus, Aspergillus, Aureobasidium, Auxarthron, Blastomyces, Candida, Chrysosporuim, Chrysosporuim Debaryomyces, Coccidiodes, Cryptococcus, Gymnoascus, Hansenula, Histoplasma, Issatchenkia, Kluyveromyces, Lipomyces, Lssatchenkia, Microsporum, Myxotrichum, Myxozyma, Oidiodendron, Pachysolen, Penicillium, Pichia, Rhodosporidium, Rhodotorula, Rhodotorula, Saccharomyces, Schizosaccharomyces, Scopulariopsis, Sepedonium, Trichosporon, or Yarrowia. In some embodiments, a suitable yeast is of the species Arachniotus flavoluteus, Aspergillus flavus, Aspergillus fumigatus, Aspergillus niger, Aureobasidium pullulans, Auxarthron thaxteri, Blastomyces dermatitidis, Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii, Chrysosporuim keratinophilum, Coccidiodes immitis, Cryptococcus albidus var. diffluens, Cryptococcus laurentii, Cryptococcus neofomans, Debaryomyces hansenii, Gymnoascus dugwayensis, Hansenula anomala, Histoplasma capsulatum, Issatchenkia occidentalis, Isstachenkia orientalis, Kluyveromyces lactis, Kluyveromyces marxianus, Kluyveromyces thermotolerans, Kluyveromyces waltii, Lipomyces lipoferus, Lipomyces starkeyii, Microsporum gypseum, Myxotrichum deflexum, Oidiodendron echinulatum, Pachysolen tannophilis, Penicillium notatum, Pichia anomala, Pichia pastoris, Pichia stipitis, Rhodosporidium toruloides, Rhodotorula glutinus, Rhodotorula graminis, Saccharomyces cerevisiae, Saccharomyces kluyveri, Schizosaccharomyces pombe, Scopulariopsis acremonium, Sepedonium chrysospermum, Trichosporon cutaneum, Trichosporon pullans, Yarrowia lipolytica, or Yarrowia lipolytica (formerly classified as Candida lipolytica). In some embodiments, a yeast is a Y. lipolytica strain that includes, but is not limited to, ATCC20362, ATCC8862, ATCC18944, ATCC20228, ATCC76982 and LGAM S(7)1 strains (Papanikolaou S., and Aggelis G., Bioresour. Technol. 82(1):43-9 (2002)). In certain embodiments, a yeast is a Candida species (i.e., Candida spp.) yeast. Any suitable Candida species can be used and/or genetically modified for production of a fatty dicarboxylic acid (e.g., octanedioic acid, decanedioic acid, dodecanedioic acid, tetradecanedioic acid, hexadecanedioic acid, octadecanedioic acid, eicosanedioic acid). In some embodiments, suitable Candida species include, but are not limited to Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii and any other Candida spp. yeast described herein. Non-limiting examples of Candida spp. strains include, but are not limited to, sAA001 (ATCC20336), sAA002 (ATCC20913), sAA003 (ATCC20962), sAA496 (US2012/0077252), sAA106 (US2012/0077252), SU-2 (ura3-/ura3-), H5343 (beta oxidation blocked; U.S. Pat. No. 5,648,247) strains. Any suitable strains from Candida spp. yeast may be utilized as parental strains for genetic modification.

Yeast genera, species and strains are often so closely related in genetic content that they can be difficult to distinguish, classify and/or name. In some cases strains of C. lipolytica and Y. lipolytica can be difficult to distinguish, classify and/or name and can be, in some cases, considered the same organism. In some cases, various strains of C. tropicalis and C. viswanathii can be difficult to distinguish, classify and/or name (for example see Arie et. al., J. Gen. Appl. Microbiol., 46, 257-262 (2000). Some C. tropicalis and C. viswanathii strains obtained from ATCC as well as from other commercial or academic sources can be considered equivalent and equally suitable for the embodiments described herein. In some embodiments, some parental strains of C. tropicalis and C. viswanathii are considered to differ in name only.

Any suitable fungus may be selected as a host microorganism, engineered microorganism or source for a heterologous polynucleotide. Non-limiting examples of fungi include, but are not limited to, Aspergillus fungi (e.g., A. parasiticus, A. nidulans), Thraustochytrium fungi, Schizochytrium fungi and Rhizopus fungi (e.g., R. arrhizus, R. oryzae, R. nigricans). In some embodiments, a fungus is an A. parasiticus strain that includes, but is not limited to, strain ATCC24690, and in certain embodiments, a fungus is an A. nidulans strain that includes, but is not limited to, strain ATCC38163.

Any suitable prokaryote may be selected as a host microorganism, engineered microorganism or source for a heterologous polynucleotide. A Gram negative or Gram positive bacteria may be selected. Examples of bacteria include, but are not limited to, Bacillus bacteria (e.g., B. subtilis, B. megaterium), Acinetobacter bacteria, Norcardia baceteria, Xanthobacter bacteria, Escherichia bacteria (e.g., E. coli (e.g., strains DH10B, Stbl2, DH5-alpha, DB3, DB3.1), DB4, DB5, JDP682 and ccdA-over (e.g., U.S. application Ser. No. 09/518,188))), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi), Megasphaera bacteria (e.g., Megasphaera elsdenii). Bacteria also include, but are not limited to, photosynthetic bacteria (e.g., green non-sulfur bacteria (e.g., Choroflexus bacteria (e.g., C. aurantiacus), Chloronema bacteria (e.g., C. gigateum)), green sulfur bacteria (e.g., Chlorobium bacteria (e.g., C. limicola), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

Cells from non-microbial organisms can be utilized as a host microorganism, engineered microorganism or source for a heterologous polynucleotide. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; mammalian cells (e.g., NIH3T3, 293, CHO, COS, VERO, C127, BHK, Per-C6, Bowes melanoma and HeLa cells); and plant cells (e.g., Arabidopsis thaliana, Nicotania tabacum, Cuphea acinifolia, Cuphea aequipetala, Cuphea angustifolia, Cuphea appendiculata, Cuphea avigera, Cuphea avigera var. pulcherrima, Cuphea axilliflora, Cuphea bahiensis, Cuphea baillonis, Cuphea brachypoda, Cuphea bustamanta, Cuphea calcarata, Cuphea calophylla, Cuphea calophylla subsp. mesostemon, Cuphea carthagenensis, Cuphea circaeoides, Cuphea confertiflora, Cuphea cordata, Cuphea crassiflora, Cuphea cyanea, Cuphea decandra, Cuphea denticulata, Cuphea disperma, Cuphea epilobiifolia, Cuphea ericoides, Cuphea flava, Cuphea flavisetula, Cuphea fuchsiifolia, Cuphea gaumeri, Cuphea glutinosa, Cuphea heterophylla, Cuphea hookeriana, Cuphea hyssopifolia (Mexican-heather), Cuphea hyssopoides, Cuphea ignea, Cuphea ingrata, Cuphea jorullensis, Cuphea lanceolata, Cuphea linarioides, Cuphea llavea, Cuphea lophostoma, Cuphea lutea, Cuphea lutescens, Cuphea melanium, Cuphea melvilla, Cuphea micrantha, Cuphea micropetala, Cuphea mimuloides, Cuphea nitidula, Cuphea palustris, Cuphea parsonsia, Cuphea pascuorum, Cuphea paucipetala, Cuphea procumbens, Cuphea pseudosilene, Cuphea pseudovaccinium, Cuphea pulchra, Cuphea racemosa, Cuphea repens, Cuphea salicifolia, Cuphea salvadorensis, Cuphea schumannii, Cuphea sessiliflora, Cuphea sessilifolia, Cuphea setosa, Cuphea spectabilis, Cuphea spermacoce, Cuphea splendida, Cuphea splendida var. viridiflava, Cuphea strigulosa, Cuphea subuligera, Cuphea teleandra, Cuphea thymoides, Cuphea tolucana, Cuphea urens, Cuphea utriculosa, Cuphea viscosissima, Cuphea watsoniana, Cuphea wrightii, Cuphea lanceolata).

Microorganisms or cells used as host organisms or source for a heterologous polynucleotide are commercially available. Microorganisms and cells described herein, and other suitable microorganisms and cells are available, for example, from Invitrogen Corporation, (Carlsbad, CA), American Type Culture Collection (Manassas, Virginia), and Agricultural Research Culture Collection (NRRL; Peoria, Illinois). Host microorganisms and engineered microorganisms may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

### Polymerase

A particularly useful function of a polymerase is to catalyze the polymerization of a nucleic acid strand using an existing nucleic acid as a template. Other functions that are useful are described elsewhere herein. Examples of useful polymerases include DNA polymerases and RNA polymerases.

The ability to improve specificity, processivity, or other features of polymerases unnatural nucleic acids would be highly desirable in a variety of contexts where, e.g., unnatural nucleic acid incorporation is desired, including amplification, sequencing, labeling, detection, cloning, and many others. The present invention provides polymerases with modified properties for unnatural nucleic acids, methods of making such polymerases, methods of using such polymerases, and many other features that will become apparent upon a complete review of the following.

In some instances, disclosed herein includes polymerases that incorporate unnatural nucleic acids into a growing template copy, e.g., during DNA amplification. In some embodiments, polymerases can be modified such that the active site of the polymerase is modified to reduce steric entry inhibition of the unnatural nucleic acid into the active site. In some embodiments, polymerases can be modified to provide complementarity with one or more unnatural features of the unnatural nucleic acids. Such polymerases can be expressed or engineered in cells for stably incorporating a UBP into the cells. Accordingly, the invention includes compositions that include a heterologous or recombinant polymerase and methods of use thereof.

Polymerases can be modified using methods pertaining to protein engineering. For example, molecular modeling can be carried out based on crystal structures to identify the locations of the polymerases where mutations can be made to modify a target activity. A residue identified as a target for replacement can be replaced with a residue selected using energy minimization modeling, homology modeling, and/or conservative amino acid substitutions, such as described in Bordo, et al. J Mol Biol 217: 721-729 (1991) and Hayes, et al. Proc Natl Acad Sci, USA 99: 15926-15931 (2002).

Any of a variety of polymerases can be used in a method or composition set forth herein including, for example, protein-based enzymes isolated from biological systems and functional variants thereof. Reference to a particular polymerase, such as those exemplified below, will be understood to include functional variants thereof unless indicated otherwise. In some embodiments, a polymerase is a wild type polymerase. In some embodiments, a polymerase is a modified, or mutant, polymerase.

Polymerases, with features for improving entry of unnatural nucleic acids into active site regions and for coordinating with unnatural nucleotides in the active site region, can also be used. In some embodiments, a modified polymerase has a modified nucleotide binding site.

In some embodiments, a modified polymerase has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid. In some embodiments, a modified or wild type polymerase has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified sugar. In some embodiments, a modified or wild type polymerase has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified base. In some embodiments, a modified or wild type polymerase has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward a nucleic acid comprising a triphosphate and/or the unnatural nucleic acid without the triphosphate. For example, a modified or wild type polymerase can have a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid with a diphosphate or monophosphate, or no phosphate, or a combination thereof.

In some embodiments, a modified or wild type polymerase has a relaxed specificity for an unnatural nucleic acid. In some embodiments, a modified or wild type polymerase has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid.

Absence of exonuclease activity can be a wild type characteristic or a characteristic imparted by a variant or engineered polymerase. For example, an exo minus Klenow fragment is a mutated version of Klenow fragment that lacks 3′ to 5′ proofreading exonuclease activity.

The method of the invention may be used to expand the substrate range of any DNA polymerase which lacks an intrinsic 3 to 5′ exonuclease proofreading activity or where a 3 to 5′ exonuclease proofreading activity has been disabled, e.g. through mutation. Examples of DNA polymerases include polA, polB (see e.g. Parrel & Loeb, Nature Struc Biol 2001) polC, polD, polY, polX and reverse transcriptases (RT) but preferably are processive, high-fidelity polymerases (PCT/GB2004/004643). In some embodiments a modified or wild type polymerase substantially lacks 3′ to 5′ proofreading exonuclease activity. In some embodiments a modified or wild type polymerase substantially lacks 3′ to 5′ proofreading exonuclease activity for an unnatural nucleic acid. In some embodiments, a modified or wild type polymerase has a 3′ to 5′ proofreading exonuclease activity. In some embodiments, a modified or wild type polymerase has a 3′ to 5′ proofreading exonuclease activity for a natural nucleic acid and substantially lacks 3′ to 5′ proofreading exonuclease activity for an unnatural nucleic acid.

In some embodiments, a modified polymerase has a 3′ to 5′ proofreading exonuclease activity that is at least about 60%, 70%, 80%, 90%, 95%, 97%, 98%, 99%, 99.5%, 99.99% the proofreading exonuclease activity of the wild type polymerase. In some embodiments, a modified polymerase has a 3′ to 5′ proofreading exonuclease activity for an unnatural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for an unnatural nucleic acid and a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to the natural nucleic acid.

In some embodiments, polymerases are characterized according to their rate of dissociation from nucleic acids. In some embodiments a polymerase has a relatively low dissociation rate for one or more natural and unnatural nucleic acids. In some embodiments a polymerase has a relatively high dissociation rate for one or more natural and unnatural nucleic acids. The dissociation rate is an activity of a polymerase that can be adjusted to tune reaction rates in methods set forth herein.

In some embodiments, polymerases are characterized according to their fidelity when used with a particular natural and/or unnatural nucleic acid or collections of natural and/or unnatural nucleic acid. Fidelity generally refers to the accuracy with which a polymerase incorporates correct nucleic acids into a growing nucleic acid chain when making a copy of a nucleic acid template. DNA polymerase fidelity can be measured as the ratio of correct to incorrect natural and unnatural nucleic acid incorporations when the natural and unnatural nucleic acid are present, e.g., at equal concentrations, to compete for strand synthesis at the same site in the polymerase-strand-template nucleic acid binary complex. DNA polymerase fidelity can be calculated as the ratio of (k_{cat}/Kₘ) for the natural and unnatural nucleic acid and (k_{cat}/Kₘ) for the incorrect natural and unnatural nucleic acid; where k_{cat} and Kₘ are Michaelis-Menten parameters in steady state enzyme kinetics (Fersht, A. R. (1985) Enzyme Structure and Mechanism, 2nd ed., p 350, W. H. Freeman & Co., New York., incorporated herein by reference). In some embodiments, a polymerase has a fidelity value of at least about 100, 1000, 10,000, 100,000, or 1×10⁶, with or without a proofreading activity.

In some embodiments, polymerases from native sources or variants thereof are screened using an assay that detects incorporation of an unnatural nucleic acid having a particular structure. In one example, polymerases can be screened for the ability to incorporate an unnatural nucleic acid or UBP; e.g., d5SICSTP, dNaMTP, or d5SICSTP-dNaMTP UBP. A polymerase, e.g., a heterologous polymerase, can be used that displays a modified property for the unnatural nucleic acid as compared to the wild-type polymerase. For example, the modified property can be, e.g., Kₘ, k_{cat}, Vₘₐₓ, polymerase processivity in the presence of an unnatural nucleic acid (or of a naturally occurring nucleotide), average template read-length by the polymerase in the presence of an unnatural nucleic acid, specificity of the polymerase for an unnatural nucleic acid, rate of binding of an unnatural nucleic acid, rate of product (pyrophosphate, triphosphate, etc.) release, branching rate, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the polymerase optionally has an increased rate of binding of an unnatural nucleic acid, an increased rate of product release, and/or a decreased branching rate, as compared to a wild-type polymerase.

At the same time, a polymerase can incorporate natural nucleic acids, e.g., A, C, G, and T, into a growing nucleic acid copy. For example, a polymerase optionally displays a specific activity for a natural nucleic acid that is at least about 5% as high (e.g., 5%, 10%, 25%, 50%, 75%, 100% or higher), as a corresponding wild-type polymerase and a processivity with natural nucleic acids in the presence of a template that is at least 5% as high (e.g., 5%, 10%, 25%, 50%, 75%, 100% or higher) as the wild-type polymerase in the presence of the natural nucleic acid. Optionally, the polymerase displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type polymerase.

Polymerases used herein that can have the ability to incorporate an unnatural nucleic acid of a particular structure can also be produced using a directed evolution approach. A nucleic acid synthesis assay can be used to screen for polymerase variants having specificity for any of a variety of unnatural nucleic acids. For example, polymerase variants can be screened for the ability to incorporate an unnatural nucleic acid or UBP; e.g., d5SICSTP, dNaMTP, or d5SICSTP-dNaMTP UBP into nucleic acids. In some embodiments, such an assay is an in vitro assay, e.g., using a recombinant polymerase variant. In some embodiments, such an assay is an in vivo assay, e.g., expressing a polymerase variant in a cell. Such directed evolution techniques can be used to screen variants of any suitable polymerase for activity toward any of the unnatural nucleic acids set forth herein.

Modified polymerases of the compositions described can optionally be a modified and/or recombinant Φ29-type DNA polymerase. Optionally, the polymerase can be a modified and/or recombinant Φ29, B103, GA-1, PZA, Φ15, BS32, M2Y, Nf, G1, Cp-1, PRD1, PZE, SF5, Cp-5, Cp-7, PR4, PR5, PR722, or L17 polymerase.

Modified polymerases of the compositions described can optionally be modified and/or recombinant prokaryotic DNA polymerase, e.g., DNA polymerase II (Pol II), DNA polymerase III (Pol III), DNA polymerase IV (Pol IV), DNA polymerase V (Pol V). In some embodiments, the modified polymerases comprise polymerases that mediate DNA synthesis across non-instructional damaged nucleotides. In some embodiments, the genes encoding Pol I, Pol II (polB), Poll IV (dinB), and/or Pol V (umuCD) are constitutively expressed, or overexpressed, in the engineered cell, or SSO. In some embodiments, an increase in expression or overexpression of Pol II contributes to an increased retention of unnatural base pairs (UBPs) in an engineered cell, or SSO.

Nucleic acid polymerases generally useful in the invention include DNA polymerases, RNA polymerases, reverse transcriptases, and mutant or altered forms thereof. DNA polymerases and their properties are described in detail in, among other places, DNA Replication 2^{nd} edition, Kornberg and Baker, W. H. Freeman, New York, N. Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (TIi) DNA polymerase (also referred to as Vent™ DNA polymerase, Cariello et al, 1991, Polynucleotides Res, 19: 4193, New England Biolabs), 9° Nm™ DNA polymerase (New England Biolabs), Stoffel fragment, Thermo Sequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al, 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al, 1981, J Biol. Chem. 256:3112), and archaeal DP1I/DP2 DNA polymerase II (Cann et al, 1998, Proc. Natl. Acad. Sci. USA 95:14250). Both mesophilic polymerases and thermophilic polymerases are contemplated. Thermophilic DNA polymerases include, but are not limited to, ThermoSequenase®, 9° Nm™, Therminator™, Taq, Tne, Tma, Pfu, TfI, Tth, TIi, Stoffel fragment, Vent™ and Deep Vent™ DNA polymerase, KOD DNA polymerase, Tgo, JDF-3, and mutants, variants and derivatives thereof. A polymerase that is a 3′exonuclease-deficient mutant is also contemplated. Reverse transcriptases useful in the invention include, but are not limited to, reverse transcriptases from HIV, HTLV-I, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al, CRC Crit Rev Biochem. 3:289-347(1975)). Further examples of polymerases include, but are not limited to 9° N DNA Polymerase, Taq DNA polymerase, Phusion® DNA polymerase, Pfu DNA polymerase, RB69 DNA polymerase, KOD DNA polymerase, and VentR® DNA polymerase Gardner et al. (2004) “Comparative Kinetics of Nucleotide Analog Incorporation by Vent DNA Polymerase (J. Biol. Chem., 279(12), 11834-11842; Gardner and Jack “Determinants of nucleotide sugar recognition in an archaeon DNA polymerase” Nucleic Acids Research, 27(12) 2545-2553.) Polymerases isolated from non-thermophilic organisms can be heat inactivatable. Examples are DNA polymerases from phage. It will be understood that polymerases from any of a variety of sources can be modified to increase or decrease their tolerance to high temperature conditions. In some embodiments, a polymerase can be thermophilic. In some embodiments, a thermophilic polymerase can be heat inactivatable. Thermophilic polymerases are typically useful for high temperature conditions or in thermocycling conditions such as those employed for polymerase chain reaction (PCR) techniques.

In some embodiments, the polymerase comprises D29, B103, GA-1, PZA, D15, BS32, M2Y, Nf, G1, Cp-1, PRD1, PZE, SF5, Cp-5, Cp-7, PR4, PR5, PR722, L17, ThermoSequenase®, 9° Nm™, Therminator™ DNA polymerase, Tne, Tma, TfI, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UlTma DNA polymerase, E. coli DNA polymerase I, E. coli DNA polymerase III, archaeal DP1I/DP2 DNA polymerase II, 9° N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some embodiments, the polymerase is DNA polymerase 1-Klenow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

Additionally, such polymerases can be used for DNA amplification and/or sequencing applications, including real-time applications, e.g., in the context of amplification or sequencing that include incorporation of unnatural nucleic acid residues into DNA by the polymerase. In other embodiments, the unnatural nucleic acid that is incorporated can be the same as a natural residue, e.g., where a label or other moiety of the unnatural nucleic acid is removed by action of the polymerase during incorporation, or the unnatural nucleic acid can have one or more feature that distinguishes it from a natural nucleic acid.

### Nucleotide Transporter

Nucleotide transporters (NTs) are a group of membrane transport proteins that facilitate nucleoside substrates across cell membranes and vesicles. In some embodiments, there are two types of nucleoside transporters, concentrative nucleoside transporters and equilibrative nucleoside transporters. In some instances, NTs also encompass the organic anion transporters (OAT) and the organic cation transporters (OCT). In some instances, nucleotide transporter is a nucleoside triphosphate transporter.

In some embodiments, a nucleotide triphosphate transporter (NTT) is from bacteria, plant, or algae. In some embodiments, a nucleotide nucleoside triphosphate transporter is TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT1 (Rickettsia prowazekii).

In some embodiments, NTT is CNT1, CNT2, CNT3, ENT1, ENT2, OAT1, OAT3, or OCT1.

In some embodiments, NTT imports unnatural nucleic acids into an organism, e.g. a cell. In some embodiments, NTTs can be modified such that the nucleotide binding site of the NTT is modified to reduce steric entry inhibition of the unnatural nucleic acid into the nucleotide biding site. In some embodiments, NTTs can be modified to provide increased interaction with one or more unnatural features of the unnatural nucleic acids. Such NTTs can be expressed or engineered in cells for stably importing a UBP into the cells. Accordingly, the invention includes compositions that include a heterologous or recombinant NTT and methods of use thereof.

NTTs can be modified using methods pertaining to protein engineering. For example, molecular modeling can be carried out based on crystal structures to identify the locations of the NTTs where mutations can be made to modify a target activity or binding site. A residue identified as a target for replacement can be replaced with a residue selected using energy minimization modeling, homology modeling, and/or conservative amino acid substitutions, such as described in Bordo, et al. J Mol Biol 217: 721-729 (1991) and Hayes, et al. Proc Natl Acad Sci, USA 99: 15926-15931 (2002).

Any of a variety of NTTs can be used in a method or composition set forth herein including, for example, protein-based enzymes isolated from biological systems and functional variants thereof. Reference to a particular NTT, such as those exemplified below, will be understood to include functional variants thereof unless indicated otherwise. In some embodiments, a NTT is a wild type NTT. In some embodiments, a NTT is a modified, or mutant, NTT.

NTTs, with features for improving entry of unnatural nucleic acids into cells and for coordinating with unnatural nucleotides in the nucleotide biding region, can also be used. In some embodiments, a modified NTT has a modified nucleotide binding site. In some embodiments, a modified or wild type NTT has a relaxed specificity for an unnatural nucleic acid.

In some embodiments, a modified NTT has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the unnatural nucleic acid. In some embodiments, a modified or wild type NTT has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type NTT toward a natural nucleic acid and/or the unnatural nucleic acid without the modified sugar. In some embodiments, a modified or wild type NTT has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type NTT toward a natural nucleic acid and/or the unnatural nucleic acid without the modified base. In some embodiments, a modified or wild type polymerase has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type NTT toward a nucleic acid comprising a triphosphate and/or the unnatural nucleic acid without the triphosphate. For example, a modified or wild type NTT can have a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type NTT toward the unnatural nucleic acid with a diphosphate or monophosphate, or no phosphate, or a combination thereof.

In some embodiments, a modified or wild type NTT has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the natural nucleic acid. In some embodiments, a modified or wild type NTT has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the natural nucleic acid. In some embodiments, a modified or wild type NTT has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the natural nucleic acid.

NTTs can be characterized according to their rate of dissociation from nucleic acids. In some embodiments a NTT has a relatively low dissociation rate for one or more natural and unnatural nucleic acids. In some embodiments a NTT has a relatively high dissociation rate for one or more natural and unnatural nucleic acids. The dissociation rate is an activity of a NTT that can be adjusted to tune reaction rates in methods set forth herein.

NTTs from native sources or variants thereof can be screened using an assay that detects importation of an unnatural nucleic acid having a particular structure. In one example, NTTs can be screened for the ability to import an unnatural nucleic acid or UBP; e.g., d5SICSTP, dNaMTP, or d5SICSTP-dNaMTP UBP. A NTT, e.g., a heterologous NTT, can be used that displays a modified property for the unnatural nucleic acid as compared to the wild-type NTT. For example, the modified property can be, e.g., Kₘ, k_{cat}, Vₘₐₓ, NTT importation in the presence of an unnatural nucleic acid (or of a naturally occurring nucleotide), average template read-length by a cell with the NTT in the presence of an unnatural nucleic acid, specificity of the NTT for an unnatural nucleic acid, rate of binding of an unnatural nucleic acid, or rate of product release, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the NTT optionally has an increased rate of binding of an unnatural nucleic acid, an increased rate of product release, and/or an increased cell importation rate, as compared to a wild-type NTT.

At the same time, a NTT can import natural nucleic acids, e.g., A, C, G, and T, into cell. For example, a NTT optionally displays a specific importation activity for a natural nucleic acid that is at least about 5% as high (e.g., 5%, 10%, 25%, 50%, 75%, 100% or higher), as a corresponding wild-type NTT. Optionally, the NTT displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type NTT.

NTTs used herein that can have the ability to import an unnatural nucleic acid of a particular structure can also be produced using a directed evolution approach. A nucleic acid synthesis assay can be used to screen for NTT variants having specificity for any of a variety of unnatural nucleic acids. For example, NTT variants can be screened for the ability to import an unnatural nucleic acid or UBP; e.g., d5SICSTP, dNaMTP, or d5SICSTP-dNaMTP UBP into nucleic acids. In some embodiments, such an assay is an in vitro assay, e.g., using a recombinant NTT variant. In some embodiments, such an assay is an in vivo assay, e.g., expressing a NTT variant in a cell. Such directed evolution techniques can be used to screen variants of any suitable NTT for activity toward any of the unnatural nucleic acids set forth herein.

### Nucleic Acid Reagents & Tools

A nucleic acid reagent for use with a method, cell, or engineered microorganism described herein comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat or mouse, for example. In some embodiments, a nucleic acid reagent or other reagent described herein is isolated or purified.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media.

A nucleic acid or nucleic acid reagent can comprise certain elements, e.g., regulatory elements, often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

Nucleic acid reagents, e.g., expression cassettes and/or expression vectors, can include a variety of regulatory elements, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleotide triphosphate transporter nucleic acid segment. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3″ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

As noted above, nucleic acid reagents may also comprise one or more 5′ UTR's, and one or more 3′UTR's. For example, expression vectors used in eukaryotic host cells (e.g., yeast, fungi, insect, plant, animal, human or nucleated cells) and prokaryotic host cells (e.g., virus, bacterium) can contain sequences that signal for the termination of transcription which can affect mRNA expression. These regions can be transcribed as polyadenylated segments in the untranslated portion of the mRNA encoding tissue factor protein. The 3″ untranslated regions also include transcription termination sites. In some preferred embodiments, a transcription unit comprises a polyadenylation region. One benefit of this region is that it increases the likelihood that the transcribed unit will be processed and transported like mRNA. The identification and use of polyadenylation signals in expression constructs is well established. In some preferred embodiments, homologous polyadenylation signals can be used in the transgene constructs.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, -35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′ UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR is used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

Expression of a nucleotide triphosphate transporter from an expression cassette or expression vector can be controlled by any promoter capable of expression in prokaryotic cells or eukaryotic cells. A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments. In some embodiments, a promoter element can be isolated from a gene or organism and inserted in functional connection with a polynucleotide sequence to allow altered and/or regulated expression. A non-native promoter (e.g., promoter not normally associated with a given nucleic acid sequence) used for expression of a nucleic acid often is referred to as a heterologous promoter. In certain embodiments, a heterologous promoter and/or a 5′UTR can be inserted in functional connection with a polynucleotide that encodes a polypeptide having a desired activity as described herein. The terms “operably linked” and “in functional connection with” as used herein with respect to promoters, refer to a relationship between a coding sequence and a promoter element. The promoter is operably linked or in functional connection with the coding sequence when expression from the coding sequence via transcription is regulated, or controlled by, the promoter element. The terms “operably linked” and “in functional connection with” are utilized interchangeably herein with respect to promoter elements.

A promoter often interacts with a RNA polymerase. A polymerase is an enzyme that catalyzes synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologous promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide or protein.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional or internal signals frequently are influenced by a signal (direct or indirect) that binds at or near the promoter and increases or decreases expression of the target sequence under certain conditions.

Non-limiting examples of selective or regulatory agents that influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like). In some embodiments, the regulatory or selective agent can be added to change the existing growth conditions to which the organism is subjected (e.g., growth in liquid culture, growth in a fermenter, growth on solid nutrient plates and the like for example).

In some embodiments, regulation of a promoter element can be used to alter (e.g., increase, add, decrease or substantially eliminate) the activity of a peptide, polypeptide or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologous promoter operably linked to a nucleotide sequence of interest, in certain embodiments.

Nucleic acids encoding heterologous proteins, e.g., nucleotide triphosphate transporters, can be inserted into or employed with any suitable expression system. In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids).

Recombinant expression is usefully accomplished using an expression cassette that can be part of a vector, such as a plasmid. A vector can include a promoter operably linked to nucleic acid encoding a nucleotide triphosphate transporter. A vector can also include other elements required for transcription and translation as described herein. An expression cassette, expression vector, and sequences in a cassette or vector can be heterologous to the cell to which the unnatural nucleotides are contacted. For example, a nucleotide triphosphate transporter sequence can be heterologous to the cell.

A variety of prokaryotic and eukaryotic expression vectors suitable for carrying, encoding and/or expressing nucleotide triphosphate transporters can be produced. Such expression vectors include, for example, pET, pET3d, pCR2.1, pBAD, pUC, and yeast vectors. The vectors can be used, for example, in a variety of in vivo and in vitro situations. Non-limiting examples of prokaryotic promoters that can be used include SP6, T7, T5, tac, bla, trp, gal, lac, or maltose promoters. Non-limiting examples of eukaryotic promoters that can be used include constitutive promoters, e.g., viral promoters such as CMV, SV40 and RSV promoters, as well as regulatable promoters, e.g., an inducible or repressible promoter such as a tet promoter, a hsp70 promoter, and a synthetic promoter regulated by CRE. Vectors for bacterial expression include pGEX-5X-3, and for eukaryotic expression include pCIneo-CMV. Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in Verma, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

### Cloning

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described herein. In some embodiments, a cloning strategy can be combined with genetic manipulation such as recombination (e.g., recombination of a nucleic acid reagent with a nucleic acid sequence of interest into the genome of the organism to be modified, as described further herein). In some embodiments, the cloned ORF(s) can produce (directly or indirectly) modified or wild type nucleotide triphosphate transporters and/or polymerases), by engineering a microorganism with one or more ORFs of interest, which microorganism comprises altered activities of nucleotide triphosphate transporter activity or polymerase activity.

A nucleic acid may be specifically cleaved by contacting the nucleic acid with one or more specific cleavage agents. Specific cleavage agents often will cleave specifically according to a particular nucleotide sequence at a particular site. Examples of enzyme specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, BsaI, Bsm I, BsmBI, BssH II, BstE II, Cfo I, CIa I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, SSp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N₆-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., Sauer, Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. Nos. 09/517,466, and 09/732,914; U.S. Patent Publication No. US2002/0007051; and Landy, Curr. Opin. Biotech. 3:699-707 (1993)).

Examples of recombinase cloning nucleic acids are in Gateway® systems (Invitrogen, California), which include at least one recombination site for cloning desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A nucleic acid reagent sometimes contains one or more origin of replication (ORI) elements. In some embodiments, a template comprises two or more ORIs, where one functions efficiently in one organism (e.g., a bacterium) and another function efficiently in another organism (e.g., a eukaryote, like yeast for example). In some embodiments, an ORI may function efficiently in one species (e.g., S. cerevisiae, for example) and another ORI may function efficiently in a different species (e.g., S. pombe, for example). A nucleic acid reagent also sometimes includes one or more transcription regulation sites.

A nucleic acid reagent, e.g., an expression cassette or vector, can include nucleic acid sequence encoding a marker product. A marker product is used to determine if a gene has been delivered to the cell and once delivered is being expressed. Example marker genes include the E. coli lacZ gene which encodes β-galactosidase and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern et al., J. Molec. Appl. Genet. 1: 327 (1982)), mycophenolic acid, (Mulligan et al., Science 209: 1422 (1980)) or hygromycin, (Sugden, et al., Mol. Cell. Biol. 5: 410-413 (1985)).

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one functions efficiently in one organism, and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent can be of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (e.g., Biochemistry 1996 Jun. 25; 35(25):8429-38). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some embodiments, a kit includes a suitable packaging material to house the contents of the kit. In some cases, the packaging material is constructed by well-known methods, preferably to provide a sterile, contaminant-free environment. The packaging materials employed herein can include, for example, those customarily utilized in commercial kits sold for use with nucleic acid sequencing systems. Exemplary packaging materials include, without limitation, glass, plastic, paper, foil, and the like, capable of holding within fixed limits a component set forth herein.

The packaging material can include a label which indicates a particular use for the components. The use for the kit that is indicated by the label can be one or more of the methods set forth herein as appropriate for the particular combination of components present in the kit. For example, a label can indicate that the kit is useful for a method of synthesizing a polynucleotide or for a method of determining the sequence of a nucleic acid.

Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like.

It will be understood that not all components necessary for a particular reaction need be present in a particular kit. Rather one or more additional components can be provided from other sources. The instructions provided with a kit can identify the additional component(s) that are to be provided and where they can be obtained.

In some embodiments, a kit is provided that is useful for stably incorporating an unnatural nucleic acid into a cellular nucleic acid, e.g., using the methods provided by the present invention for preparing genetically engineered cells. In one embodiment, a kit described herein includes a genetically engineered cell and one or more unnatural nucleic acids. In another embodiment, a kit described herein includes an isolated and purified plasmid comprising a sequence selected from SEQ ID NOS1-32.

In additional embodiments, the kit described herein provides a cell and a nucleic acid molecule containing a heterologous gene for introduction into the cell to thereby provide a genetically engineered cell, such as expression vectors comprising the nucleic acid of any of the embodiments hereinabove described in this paragraph.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### EXAMPLES

These examples are provided for illustrative purposes only and not to limit the scope of the claims provided herein.

### Example 1: Determination of how Cells Retain or Lose the UBP in E. coli

Under steady-state conditions, DNA containing the dNaM-dTPT3 UBP was replicated in vitro with an efficiency approaching that of a fully natural counterpart; however, these rates are likely limited by product dissociation. In vivo replication is more processive, and correspondingly less likely to be limited by product dissociation. Therefore, replication of DNA containing the UBP in the SSO may be less efficient than that of fully natural DNA, and in turn, may cause replication forks to stall. Additionally, structural studies have indicated that the UBP adopts a Watson-Crick-like structure during triphosphate insertion, but once inserted, the UBP adopts a cross-strand intercalated structure that induces local helix distortions.^{8,9} Cells interpret both stalled replication forks and helix distortions as signs of DNA damage and initiate programs to repair or tolerate the offending nucleotides, which we suspected might contribute to UBP loss.

To determine how cells retain or lose the UBP, the effects of disabling these pathways was studied. Results indicate that neither nucleotide excision repair (NER) nor the SOS response contribute significantly to UBP retention or loss. Conversely, the normal replisome polymerase, DNA polymerase III (Pol III), Pol II, and methyl-directed mismatch repair (MMR), all contribute to UBP retention; while recombinational repair (RER) of replication forks that stall provides the major route to UBP loss. Next, the replisome of the SOO was reprogramed to impart it with the ability to not only better retain the UBP on a plasmid, but also to stably harbor a UBP in its chromosome.

Nucleotide Excision Repair does not Contribute to UBP Retention or Loss

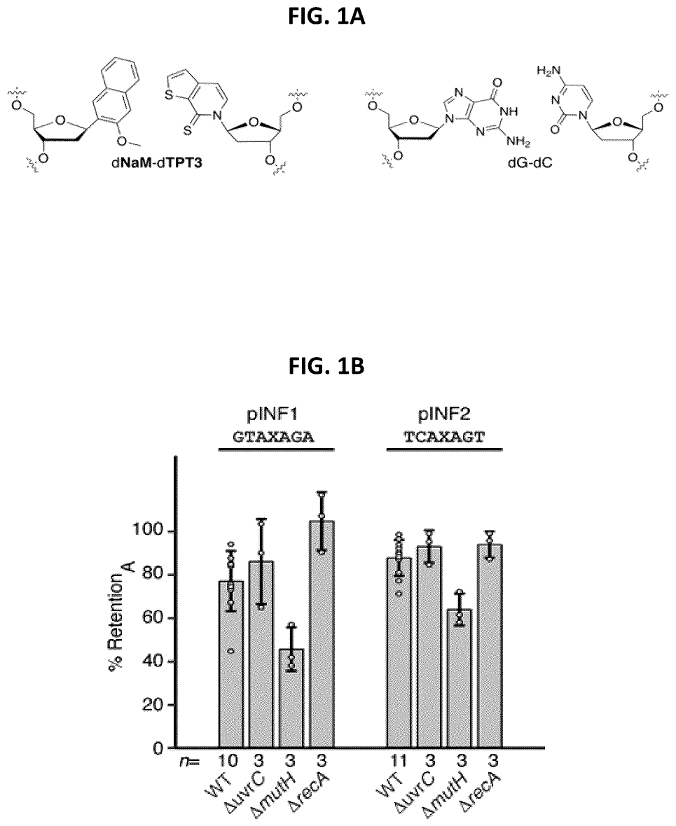

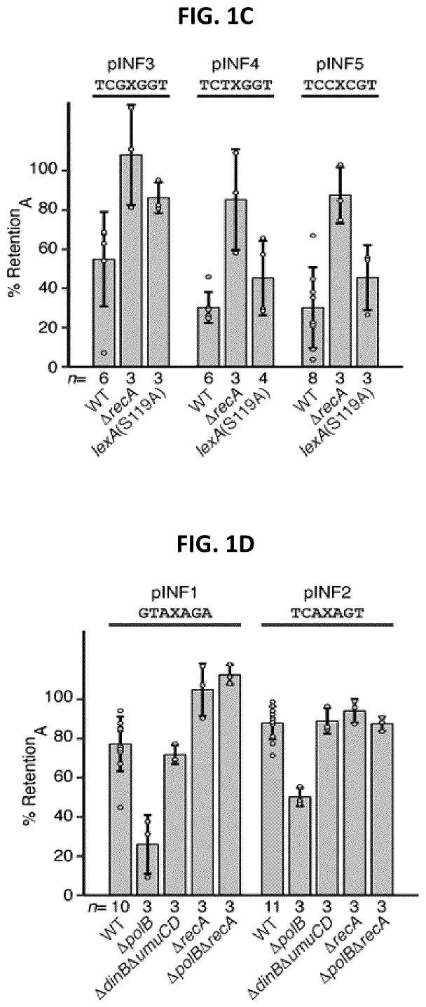

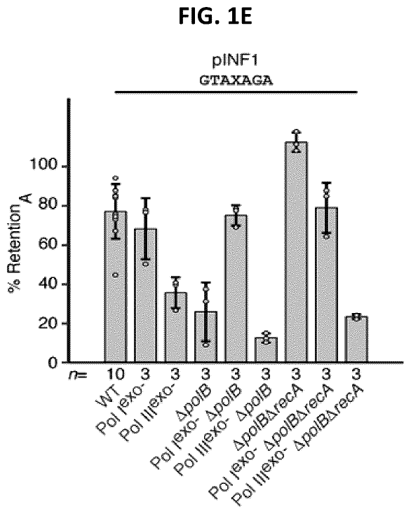

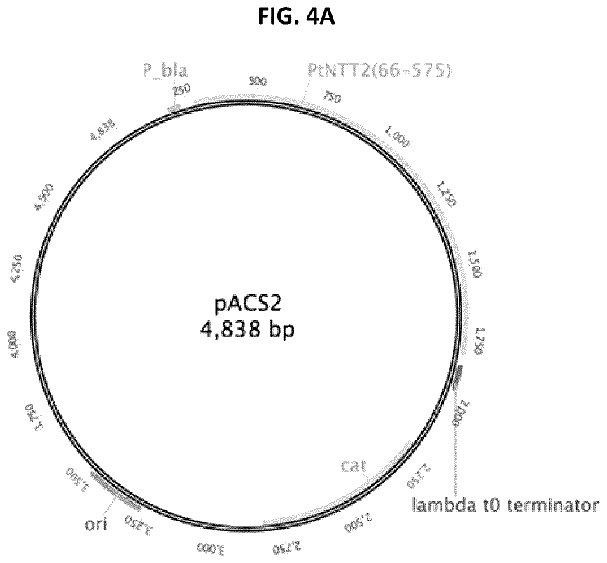

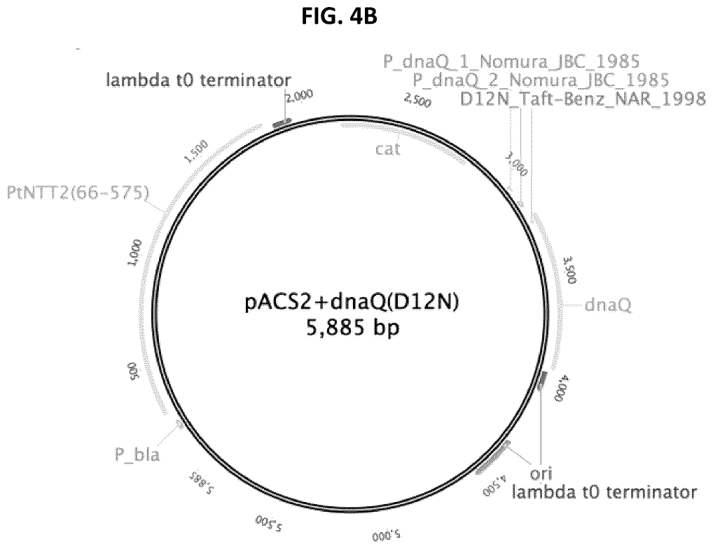

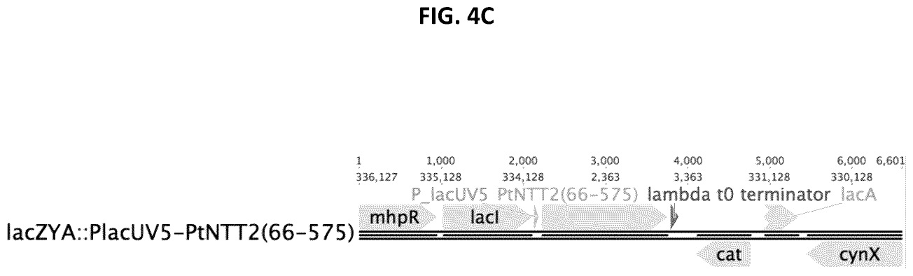

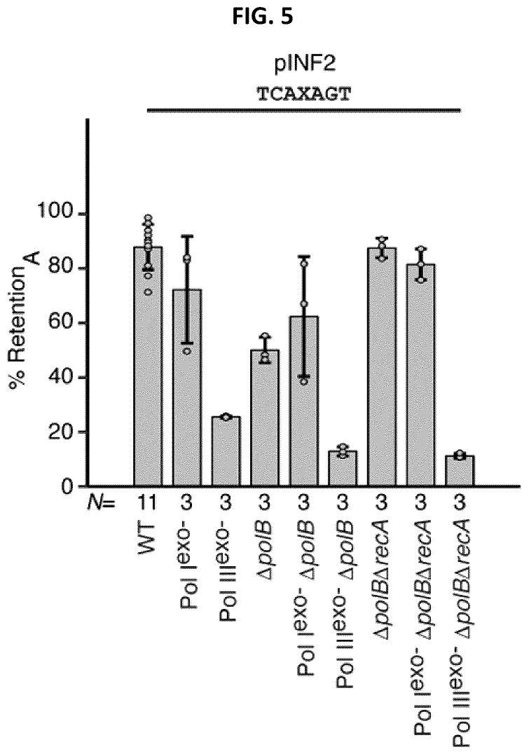

Generally, E. coli responds to DNA damage via direct damage reversal, base excision repair, NER, MMR, RER, and the SOS response. Neither direct damage reversal nor base excision repair is likely to contribute to UBP retention or loss, because these pathways rely on enzymes that recognize specific forms of DNA damage which are not likely to be mimicked by the UBP. In contrast, NER, MMR, RER, and the SOS response, are induced by less structure-specific signals. To begin to explore how cells manage to retain the UBP in their DNA, NER, which is mediated in a replication-independent manner by a complex of proteins that scan DNA for distortions resulting from bulky lesions that may be mimicked by the UBP, was studied. Contributions of NER to UBP retention or loss were explored by deleting uvrC, which encodes an essential component of NER, from the parental SSO (E. coli BL21(DE3)+pACS2 (FIG. 4)). Replication of DNA containing the dNaM-dTPT3 UBP positioned in two different sequence contexts in plasmids pINF1 and pINF2 was unaffected by deletion of uvrC, indicating that NER makes no contribution to UBP retention or loss (FIG. 1B).

### Methyl-Directed Mismatch Repair Increases UBP Retention

MMR was investigated next, which provides the critical first check of newly synthesized DNA as it emerges from a DNA polymerase during replication and is mediated by a complex of proteins that recognizes helix distortions caused by mismatched natural nucleotides. Upon detection of a mismatch, the MMR complex nicks the newly synthesized, unmethylated strand, which in turn leads to gap formation and subsequent resynthesis of the DNA. In contrast to NER, deactivation of MMR via deletion of mutH resulted in a reduction in UBP retention with both pINF1 and pINF2 (FIG. 1B). These results indicate that the helix distortions associated with the UBP are not sufficiently severe to activate MMR or that the unnatural nucleotides cannot be excised, but that the distortions caused by the pairing of an unnatural and a natural nucleotide are recognized and processed by MMR. Thus, MMR appears to effectively recognize the UBP as natural-like and selectively removes mispaired natural nucleotides, thereby supporting the stable expansion of the genetic alphabet.

### Recombinational Repair Provides the Major Route to UBP Loss

RER is mediated by RecA, which forms filaments on single-stranded DNA ahead of stalled replication forks, in turn, facilitating the formation of recombination intermediates and switching to a homologous template for continued DNA replication. The SOS response is induced when the same RecA filaments promote cleavage of the SOS repressor LexA, which leads to the derepression of a variety of genes involved in the tolerance and/or repair of the damaged DNA that caused the fork to stall. We explored the combined contribution of RER and the SOS response through the deletion of recA and observed a significant increase in UBP retention with pINF1 (FIG. 1B). To further explore the contribution of RecA, retention of the UBP in the more challenging sequences provided by pINF3, pINF4, and pINF5, was measured in the ΔrecA SSO (FIG. 1C). In these sequence contexts, the absence of recA resulted in a more dramatic increase in UBP retention.

To discern if recA deletion facilitates UBP retention by ablating RER or by preventing the induction of the SOS response, an SSO that is unable to induce the SOS response, but which is competent for RER (SSO lexA(S119A)) was examined (FIG. 1C). While selective suppression of the SOS response resulted in moderately increased UBP retention with pINF3, the increase was less than that observed with the ΔrecA SSO. With pINF4 and pINF5, selective SOS suppression resulted in only modest increases in UBP retention that were well below those observed with the recA SSO. These results demonstrate that the majority of UBP loss mediated by RecA occurs via RER and not via induction of the SOS response.

### Pol II Contributes to the Replication of DNA Containing the UBP

While the data suggests that much of UBP loss is mediated via RER, the marginal and sequence-specific increase in UBP retention with the lexA(S119A) SSO suggests that one or more SOS regulated proteins may also contribute. The contribution of the three SOS-regulated DNA polymerases, Pol II, Pol IV, and Pol V was investigated. Indeed, Pol IV and Pol V are “translesion” polymerases that are well known for their ability to mediate DNA synthesis across “non-instructional” damaged nucleotides. However, deletion of both dinB and umuCD (which encode Pol IV and the precursor of Pol V, respectively) did not impact UBP retention with either pINF1 or pINF2 (FIG. 1D). In contrast to the ΔdinBΔumuDC SSO, the deletion of polB (which encodes Pol II) resulted in a dramatic increase in UBP loss with both pINF1 and pINF2 (FIG. 1D). Overall, these data demonstrate that RER constitutes the major route to UBP loss and that Pol II provides an important route to UBP retention. While the production of Pol II is increased by the induction of SOS, the data suggests that its beneficial role is overwhelmed by the deleterious effects of the concomitantly induced RER.

### DNA Polymerases III Also Contributes to the Replication of DNA Containing the UBP

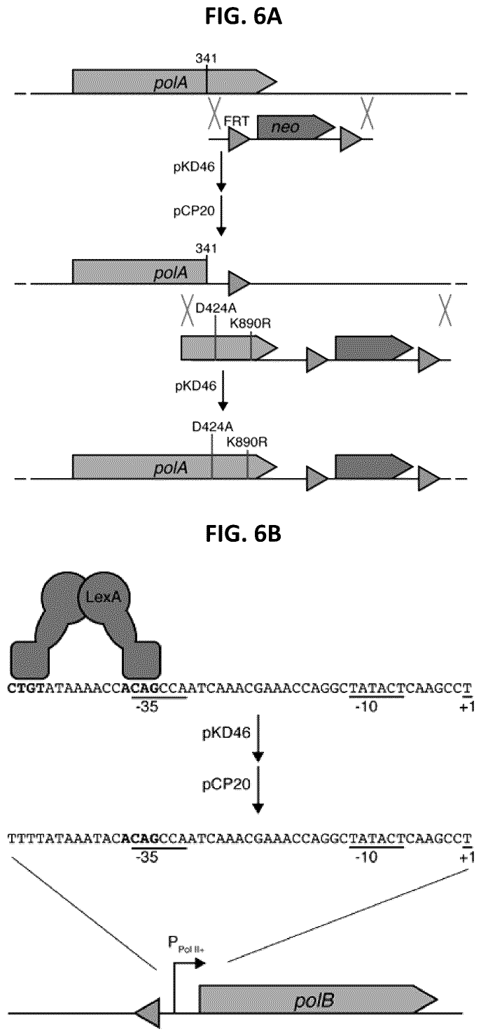

The reduced but still detectable retention of the UBP in the ΔpolB SSO, along with the negligible effects of deleting the genes encoding Pol IV and Pol V, strongly suggest that one or both of the remaining DNA polymerases, Pol I and Pol III, must also contribute to retention of the UBP. To specifically examine whether Pol I or Pol III contribute to the replication of DNA containing the UBP, we constructed and characterized strains in which their 3′-5′ exonuclease (“proofreading”) activity was eliminated or impaired via mutation (Pol I^{exo-}, polA(D424A,K890R) and Pol III^{exo-}, dnaQ(D12N), respectively (FIG. 4 and FIG. 6). While the deletion of Pol I exonuclease activity had no effect on UBP retention, the Pol III exonuclease deficient mutant showed a dramatic reduction in UBP retention. This data clearly indicates that in wild type cells, Pol III, but not Pol I, contributes to the replication of DNA containing the UBP.

To determine if any effects of the Pol I or Pol III mutations were masked by the activities of Pol II and/or RER, UBP retention was examined in the ΔpolB, or ΔpolBΔrecA SSO. Results indicate that the UBP was well retained with the ΔpolBΔrecA SSO, demonstrating that polymerases other than Pol II are capable of mediating high-level UBP retention in the absence of competition with RER-mediated loss (FIG. 1D). The Pol III exonuclease mutant again showed decreased UBP retention in both ΔpolB and ΔpolBΔrecA SSOs. However, in contrast to wild type cells, the deletion of Pol I exonuclease activity had significant and opposite effects with the ΔpolB and ΔpolBΔrecA SSO, in which retention increased and decreased, respectively. These data demonstrate that in addition to Pol II, Pol III contributes to the retention of the UBP, and in the absence of RER, Pol I does as well.

### A Model for the Replication of DNA Containing the UBP

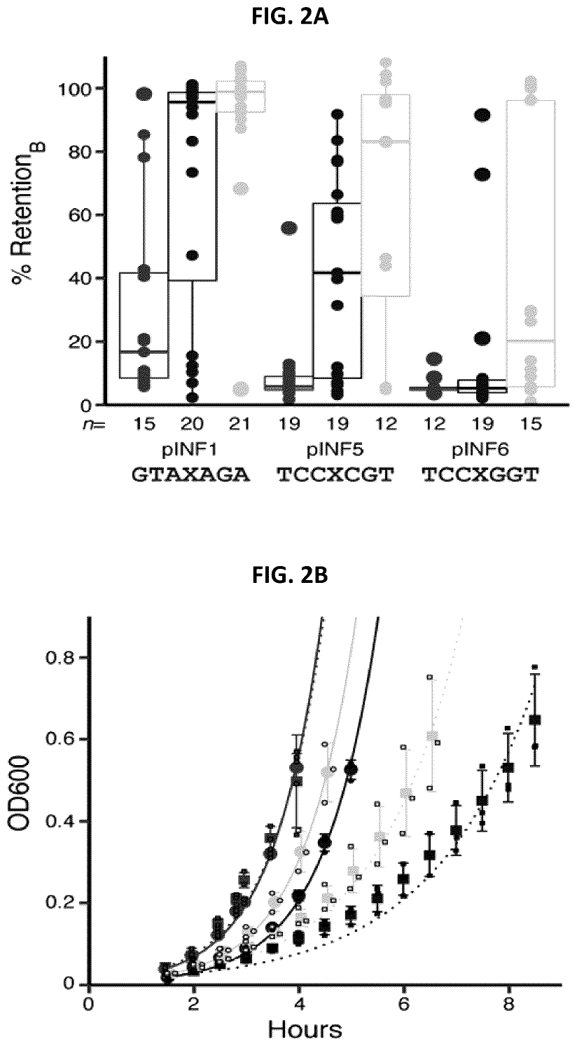

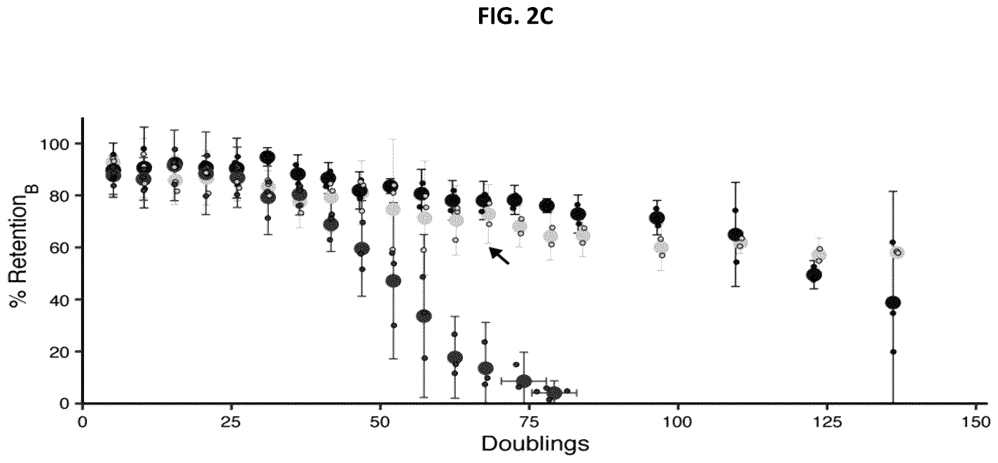

Without wishing to be bound by any particular theory, the results described herein suggest the following model for replication of DNA containing the dNaM-dTPT3 UBP in the E. coli SSO (FIG. 2). When the replisome with Pol III encounters an unnatural nucleotide during processive leading or lagging strand replication, Pol III incorporates either a natural or an unnatural nucleotide. If a natural nucleotide is incorporated, the rate of proofreading is competitive with, and perhaps more efficient than continued extension, and thus the natural nucleotide is commonly excised via the proofreading activity of Pol III. However, if a correct UBP is synthesized, more efficient extension prevents excision and the replisome continues synthesizing DNA. As it exits the polymerase, the nascent duplex is scanned by the MMR complex, which further increases UBP retention by preferentially eliminating any mispaired natural nucleotides that escaped proofreading.

Because extension of even a correct UBP is likely to be less efficient than natural synthesis, Pol III may also dissociate. The stalled fork, likely with the extending strand terminated immediately before the unnatural nucleotide in the template, is a now a substrate for RER, which reinitiates synthesis using a homologous natural sequence and thus provides the dominant mechanism for UBP loss. However, in competition with RecA-mediated RER, Pol II can rescue the stalled fork and reinitiate synthesis with high UBP retention, after which it presumably yields to Pol III and the reestablishment of a normal replication fork. The contribution of Pol I is more complex. In wild type cells, Pol I does not appear to contribute to the replication of DNA containing the UBP. In contrast, in the absence of Pol II and RecA, Pol I does contribute and correspondingly, the deletion of Pol I exonuclease activity results in decreased UBP retention. However, if the exonuclease activity is eliminated, Pol I can contribute if Pol II is eliminated, and in this case it increases retention by competing with RER.

It is accepted that Pol II has two putative roles: (1) in replication restart where Pol II rescues stalled forks after Pol III synthesizes a mispair that it cannot efficiently extend; and (2) Pol II is to compete with RER to fill in gaps created by NER as part of the cellular response to interstrand cross-linked DNA. Interestingly, the evoked role of Pol II in rescuing replication forks stalled at the UBP in competition with RER is strikingly similar to aspects of both of the putative natural roles. However, this effect on the replication of DNA containing the UBP is the most significant phenotype ever observed with its elimination.

### Optimization of the SSO

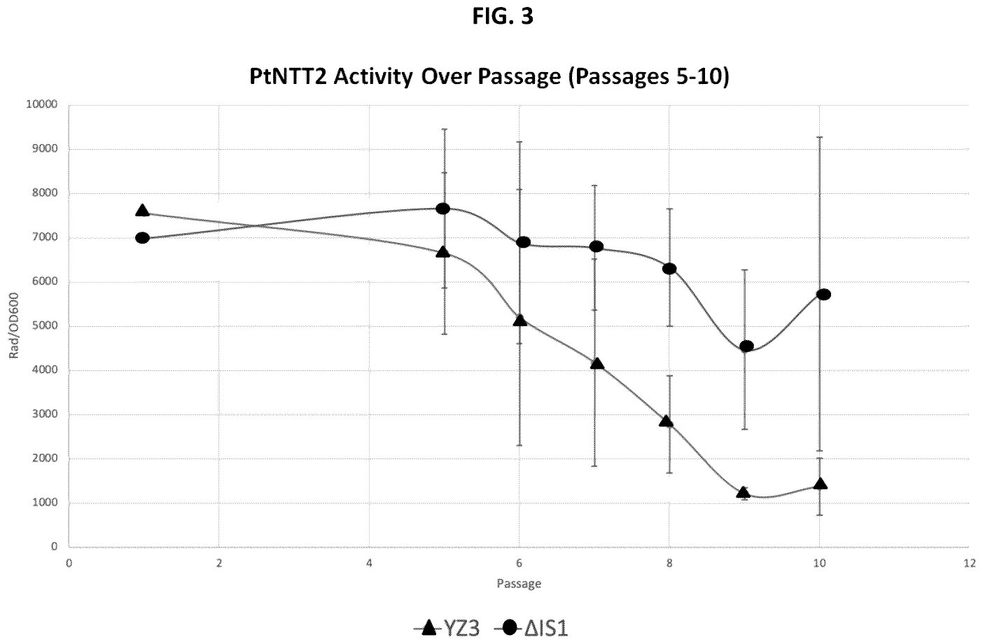

UBP retention might be optimized through the manipulation of RecA and Pol II. To explore this possibility, SSOs were optimized lacking recA and with or without Pol II constitutively expressed at SOS-derepressed levels (ΔrecA and Pol II+ΔrecA, respectively (FIG. 6). These strains (YZ3) also expressed an optimized PtNTT2 transporter from a chromosomal locus (ΔlacZYA::P_{lacUV5}-PtNTT2(66-575)) (FIG. 4). For comparison, the wild type strain with the same chromosomally integrated transporter (WT-Opt) was used. SSOs were transformed with pINF1, pINF5, or pINF6 (FIG. 3A), with pINF6 embedding the UBP in a sequence where its retention is particularly challenging, and plasmids were recovered from individual colonies to characterize UBP retention. In this case, selection on solid growth media was introduced to allow for analysis of UBP retention in individual clones, as opposed to the average UBP retentions determined in the previous experiments. A distribution of UBP retentions was observed with each plasmid in all SSOs, however, the distributions were shifted toward higher retention with the ΔrecA-Opt and especially the Pol II⁺ΔrecA SSOs, compared to the WT-Opt SSO. Additionally, only the Pol II+ΔrecA SSO produced clones with undetectable UBP loss in each sequence context examined. Notably, this was even true with pINF6, for which retention in the wild type SSO was undetectable, and only moderate (<60%) when enforced with Cas9 selection.

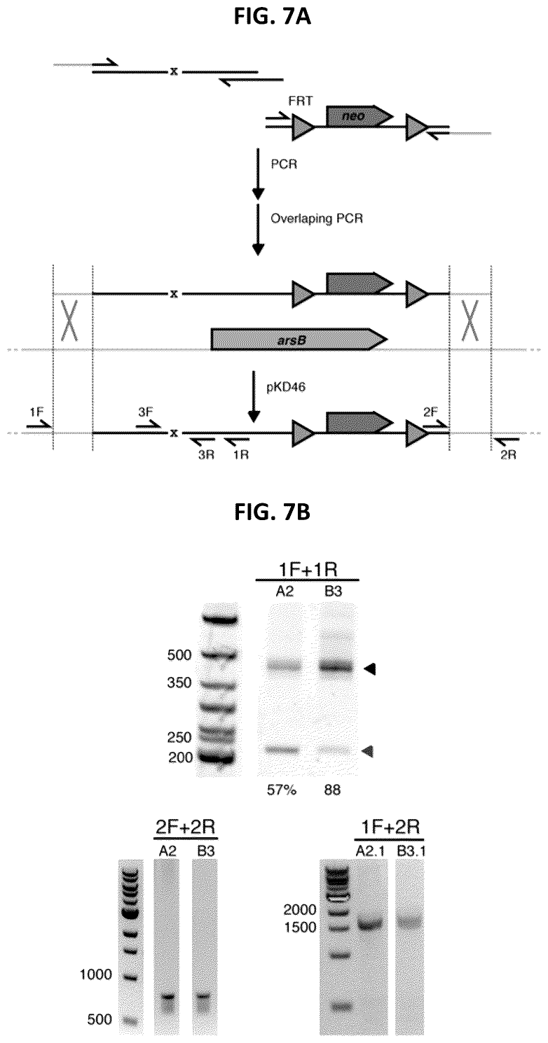

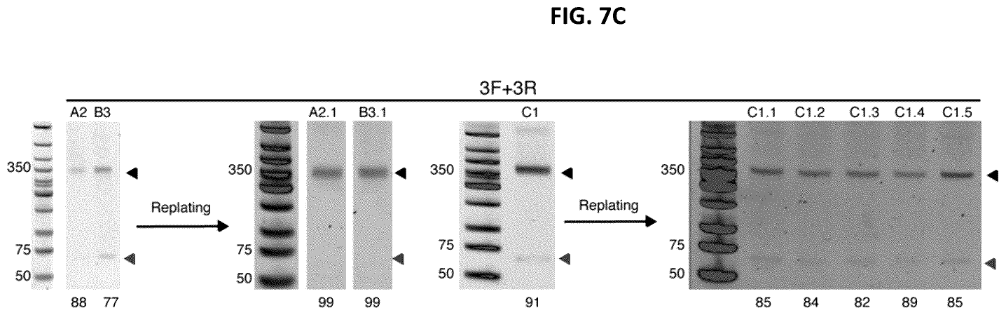

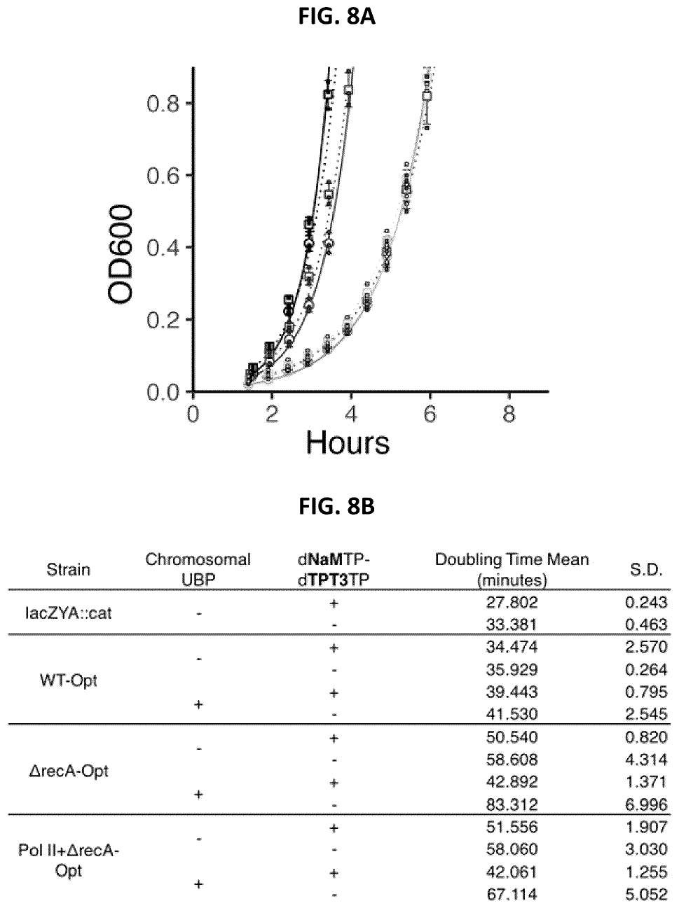

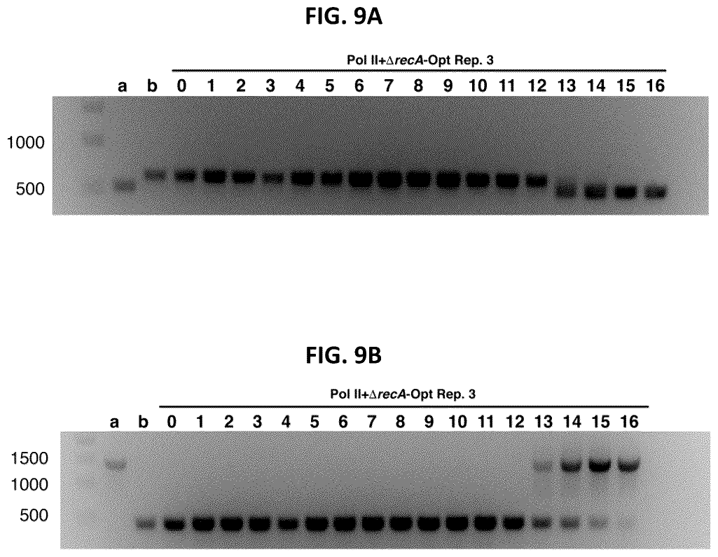

Whether the genetically optimized ΔrecA-Opt and Pol II+ΔrecA SSOs could facilitate integration of the UBP into the chromosome was evaluated. A n integration cassette was constructed that targets the sequence GTAXTGA (X=NaM) to the arsB locus, and used lambda red recombineering to integrate the cassette into the chromosomes of the WT-Opt, ΔrecA-Opt, and Pol II⁺ΔrecA SSOs. Screening of integrants for UBP retention identified clones with 100% retention from the ΔrecA-Opt and Pol II⁺ΔrecA SSOs, but despite significant effort we were unable to isolate WT-Opt clones with greater than 91% UBP retention (FIG. 7), suggesting that significant UBP loss occurred during the required growth step. To characterize the effect of the chromosomally integrated UBP, aliquots of mid-log phase cells were inoculated into growth media with or without dNaMTP and dTPT3TP (FIG. 3B, FIG. 8). The ΔrecA-Opt and Pol II⁺ΔrecA integrants grew poorly when the unnatural triphosphates were not provided, consistent with the model that RER is required to efficiently bypass an unnatural nucleotide in the template. However, this growth defect was almost entirely eliminated in both SSOs when dNaMTP and dTPT3TP were provided. Thus, the deletion of recA and the overexpression of Pol II facilitate high-level retention of the UBP in the chromosome with only minimal consequence to fitness.

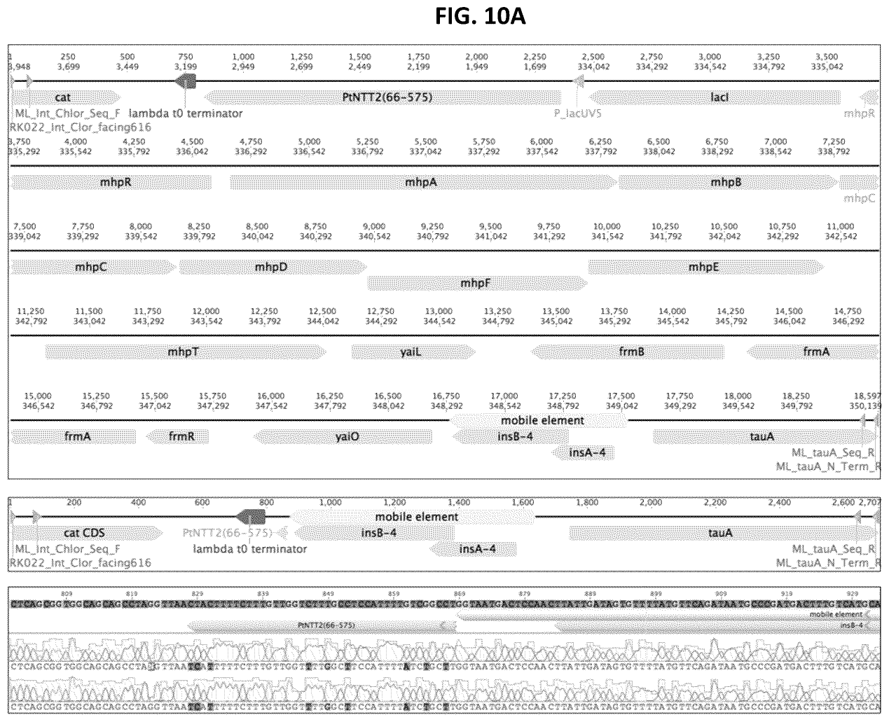

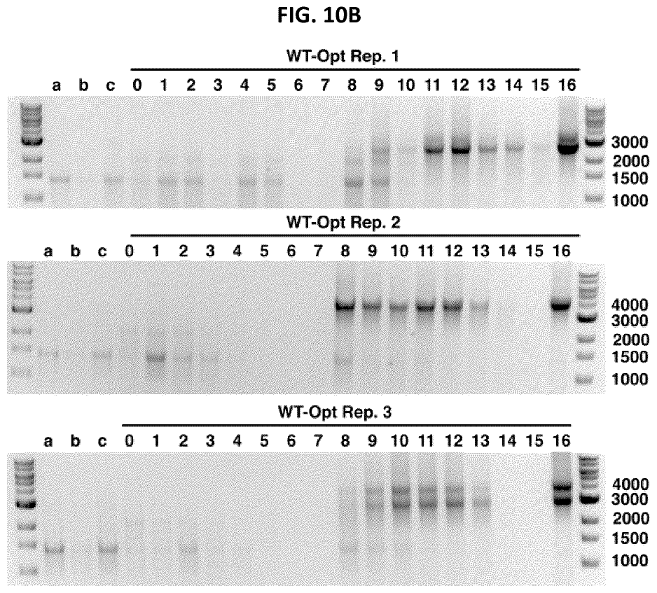

Finally, whether the genetically optimized strains facilitate the long-term stability of the chromosomally integrated UBP was evaluated. Previous studies have demonstrated that without Cas9-mediated selection for retention, a plasmid-borne UBP is lost during extended growth. The WT-Opt, ΔrecA-Opt, and Pol II⁺ΔrecA integrants were serially passaged over many generations of growth and UBP retention characterized (FIG. 3C). With WT-Opt, the UBP was slowly lost until approximately the 40^{th} generation, and then lost more rapidly with complete loss observed by the 90^{th} generation. The apparently biphasic kinetics of loss suggest that at least one additional process contributes in addition to RER. Indeed, sequencing revealed a gross chromosomal rearrangement that eliminated the PtNTT2 gene at the time of the precipitous drop in UBP retention (FIG. 10). In contrast to WT-Opt, both the ΔrecA-Opt and Pol II⁺ΔrecA SSOs, the PtNTT2 remained intact and retention of the genomic UBP remained high, especially with the Pol II⁺ΔrecA SSO, where it remained>55% after 137 generations.

These results demonstrate that not only does recA deletion facilitate UBP retention during replication, it significantly increases transporter stability during extended growth. The observed retention corresponds to a fidelity per doubling in excess of 99.6%, which in turn corresponds to loss of the chromosomal UBP in only a small fraction of the cells (<0.4%) per doubling. Thus, along with the Cas9-error elimination system, which was not employed in the current work, this error prevention system should allow for the retention of the UBP in a wide range of sequence contexts, which in turn should enable the storage of the entirety of the new information made possible by the UBP.

Since the last common ancestor of all life on earth, biological information has been stored in a four-letter alphabet. The reprogramed replisome of the Pol II⁺ΔrecA SSO represents significant progress toward the unrestricted expansion of this alphabet, and the first progress mediated through the optimization of the cell itself. While the primary goals of the research were to understand how the UBP is replicated and to use that information to optimize the SSO, the results also provide a novel route to the study of how challenging replication is normally managed. For example, while the data suggests that a significant fraction of the DNA containing the UBP is replicated by Pol III, it also clearly reveals that a significant amount is not, and in these cases, the data reveal an interesting competition between Pol II-mediated replication restart and RecA-mediated RER. Such competitions may be common during challenging replication, which may have contributed to the challenges in identifying the normal roles of Pol II. Moreover, the inability of MMR to recognize the UBP suggests that helix distortions alone are insufficient and that the process requires specific interactions with the nucleobases that are not available with the unnatural nucleotides. Finally, the increased genetic stability afforded by deletion of recA may also have significant implications for methods directed at expansion of the genetic code via amber suppression, as these methods also suffer from genetic instability with extended growth.²³ Regardless of these interesting issues, the reprogrammed SSO now allows for the more stable retention of increased biological information, including within its chromosome, and with the previous demonstration that this information can be retrieved in the form of proteins with non-canonical amino acids, should provide a platform to achieve the central goal of synthetic biology—the creation of life with new forms and functions.

### Example 2. Methods and Materials

pINF/UBP Containing DNA Construction

pINFs (Figure S8) were constructed through Golden Gate assembly of pUCX2 and insert dsDNA containing a dNaM-dTPT3 pair as described previously³ with the following modifications. UBP containing dsDNA was produced with a 50-μL PCR with chemically synthesized UBP containing oligonucleotides (0.025 ng/μL), primers introducing BsaI sites and vector homology (1 μM, Table S1), dTPT3TP (100 μM), dNaMTP (100 μM), dNTPs (200 μM), MgSO₄ (1.2 mM), OneTaq DNA Polymerase (0.025 U/μL), and OneTaq Standard Reaction Buffer (1×, New England Biolabs). The reaction was cycled through the following temperature regime on an MJ Research PTC-200 system (time in mm:ss): [94° C. 00:30|25×(94° C. 00:30|47° C. 00:30|68° C. 04:00)]. The resulting UBP containing dsDNA was purified using a DNA Clean & Concentrator-5 (Zymo Research) according to manufacturer recommendations. For pINF assembly, pUCX2 (1 μg) and insert DNA were combined at a 1:4 molar ratio in a 80 μL reaction with ATP (1 mM), T4 DNA ligase (6.65 U/μL, New England Biolabs), BsaI-HF (0.66 U/μL, New England Biolabs), and CutSmart Buffer (1×, New England Biolabs) and subjected to the following temperature regime: [37° C. 20 min|40×(37° C. 5 min|16° C. 10 min|22° C. 5 min)|37° C. 20 min|50° C. 15 min|70° C. 30 min]. BsaI-HF (0.33 U/μL) and T5 exonuclease (0.16 U/μL, New England Biolabs) were then added, and the reaction was incubated at 37° C. for 1 h to remove any pUCX2 without an insert. This reaction was purified using a DNA Clean & Concentrator-5 according to manufacturer recommendations except that reactions were mixed with 3 volumes of 1:1 DNA Wash:DNA Binding Buffer before binding to the silica column.

The UBP knock-in cassette for the arsB locus (Figure S4) was produced through overlapping PCR of a 150-bp dsDNA containing a UBP and the kanamycin resistance gene of pKD13. The 150 bp DNA was produced with a 50-μL PCR using the same reaction solution conditions as above and the following temperature regime (time in mm:ss): [98° C. 02:00|5×(98° C. 00:10|50° C. 00:10|68° C. 04:00)|15×(98° C. 00:10|58° C. 00:10|68° C. 04:00)]. The kanamycin resistance gene amplicon was produced through PCR amplification off pKD13 using Q5 DNA polymerase as per manufacturer recommendations. The amplification of long DNAs (approximately 200 bp or longer) is inhibited by the presence of dTPT3TP. Therefore, the overlap assembly PCR of the UBP containing amplicon and kanamycin resistance gene amplicon was performed on large-scale (2 mL of reaction mixture split into 40 individual 50-μL reactions) with the following solution conditions: UBP containing amplicon (0.02 ng/μL), kanamycin resistance gene amplicon (0.02 ng/μL), primers (1 μM, Table S1), dTPT3TP (5 μM), dNaMTP (100 μM), dNTPs (200 μM), MgSO₄ (1.2 mM), OneTaq DNA Polymerase (0.025 U/μL), and OneTaq Standard Reaction Buffer (1×). The reactions were subjected to the following temperature regime (time in mm:ss): [98° C. 02:00|5× (98° C. 00:10|50° C. 00:10|68° C. 04:00)|15× (98° C. 00:10|58° C. 00:10|68° C. 04:00)]. These reactions were pooled and concentrated using a DNA Clean & Concentrator-5 according to manufacturer recommendations.

### In Vivo UBP Replication in Genetic Knockouts

All genetic knockouts (FIG. 1 and Figure S2) were assayed for their ability to replicate pINEF-borne UBPs according to the following protocol. Electrocompetent cells were prepared from a 45-mL culture of mid-log phase cells (OD₆₀₀ 0.35-0.7) by pelleting cells and washing twice with 50 mL of 4° C. sterile diH₂O. Washed cells were resuspended in 4° C. sterile diH₂O at a final OD₆₀₀ of 40-60. 50 μL of cells were mixed with 2 ng of a Golden Gate assembled pINF and transferred to an electroporation cuvette (2 mm gap, Cat. #FB102, Fisher Scientific). Electroporation was performed using a Gene Pulser II (BioRad) according to manufacturer recommendations (voltage 25 kV, capacitor 2.5 μF, resistor 200Ω). Transformed cells were diluted in 950 μL of 2×YT containing chloramphenicol (33 μg/mL) and potassium phosphate (50 mM, pH 7). 40 μL of diluted cells were further diluted into a final volume of 200 μL of 2×YT containing chloramphenicol (33 μg/mL), dTPT3TP (37.5 μM), dNaMTP (150 μM), and KPi (50 mM, pH 7), transferred to a 1.5 mL tube and allowed to recover for 1 h at 37° C. and 230 RPM. 10 μL of recovered cells were diluted into a final volume of 100 μL of 2×YT containing chloramphenicol (33 μg/mL) and ampicillin (100 μg/mL), dTPT3TP (37.5 μM), dNaMTP (150 μM) and potassium phosphate (50 mM, pH 7) in the well of a 96-well plate (Ref. #655161, Greiner Bio-One). Additionally, recovered cells were plated on 2×YT Agar (2%) containing ampicillin (100 μg/mL) and potassium phosphate (50 mM, pH 7) to estimate transformation efficiency. The 96-well and transformation efficiency plates were kept at 4° C. and 37° C. overnight (approximately 12 h), respectively. The transformation efficiency plate was inspected to ensure that all samples in the 96-well plate received at least 50 colony forming units before refrigeration. The 96-well plate was then transferred to 37° C. and 230 RPM. Cells were pelleted, decanted, and frozen after reaching 0.6-0.92 OD₆₀₀. In vivo replicated pINFs were isolated using a ZR Plasmid Miniprep-Classic kit (Zymo Research) and a 5-μg silica column (Cat. #D4003, Zymo Research) according to manufacturer recommendations and advanced to biotin-shift PCR analysis (see Supporting Information). This procedure was performed in at least triplicate for each knockout strain starting from preparation of electrocompetent cells.

It should be noted that under these conditions replicates and strains undergo a similar but not identical number of cell doublings during the pINF replication experiment. However due to the pINFs unregulated origin of replication, matching cell doublings between replicates and strains does correspond to matching the number of pINF replication events. Therefore, the data in FIGS. 1 and 3A are reported as % Retention values (see Supplementary Information for further discussion) as opposed to estimated fidelities and should be interpreted as such.

Examination of Clonal pINFs

The ability of the optimized strains to clone pINFs was assessed (FIG. 3A) as described above with the following modifications. After recovery, dilutions of the recovered culture were plated on 2×YT containing agar (2%), carbenicillin (100 μg/mL), chloramphenicol (5 μg/mL), dTPT3TP (37.5 μM), dNaMTP (150 μM), and KPi (50 mM, pH 7). Plates were incubated at 37° C. for approximately 12 h. Individual colonies were picked and transferred to 100 μL of 2×YT containing carbenicillin (100 μg/mL), chloramphenicol (5 μg/mL), dTPT3TP (37.5 μM), dNaMTP (150 μM), and KPi (50 mM, pH 7) in the well of a 96-well plate. The 96-well plate was kept at 4° C. for approximately 12 h and then transferred to 37° C. and 230 RPM. Cells were pelleted, decanted, and frozen after reaching an OD₆₀₀ of 0.6-0.9. In vivo replicated pINFs were isolated using a ZR Plasmid Miniprep-Classic kit according to manufacturer recommendations and advanced to biotin-shift PCR analysis (see Supporting Information).

It should be noted that the Pol II⁺ΔrecA strain used in these experiments (FIG. 3A) had a neo cassette at the former recA locus (P_polB(−)lexA-polB+FRT+ΔrecA+KanR+IacZYA::P_lacUV5-ΔΔ(CoOp) col 2.1, Table S1).

UBP Integration at arsB

The UBP integration cassette for the arsB locus was constructed as described above and depicted in Figure S4. Integration of this cassette was performed using standard lambda red recombineering²⁴ with the following modifications. Overnight cultures of strains (WT-Opt, ΔrecA-Opt, and Pol II⁺ΔrecA-Opt in 2×YT containing chlorampenicol (5 μg/mL), and KPi (50 mM, pH 7)) possessing pKD46 were diluted to 0.03 OD₆₀₀ in 2×YT containing ampicillin (100 g/mL), chloramphenicol (5 μg/mL), and KPi (50 mM, pH 7). Cultures were grown to approximately 0.1 OD₆₀₀ then induced with 0.4% L-(+)-arabinose and allowed to continue to grow to approximately 0.4 OD₆₀₀. Electrocompetent cells were prepared from these cultures as described above. 50 μL of electrocompetent cells were mixed with 960 ng (5 μL at 192 ng/μL) of the integration cassette described above and electroporated as described above. Transformed cells were diluted to a final volume of 1 mL of 2×YT containing chloramphenicol (5 μg/mL), dTPT3TP (37.5 μM), dNaMTP (150 μM), and KPi (50 mM, pH 7), transferred to a 1.5 mL tube, and allowed to recover for 2 h at 37° C. and 230 RPM. Cells were pelleted and resuspended in 115 μL of 2×YT containing chloramphenicol (5 μg/mL), dTPT3TP (37.5 μM), dNaMTP (150 μM), KPi (50 mM, pH 7). 15 μL samples of this cell suspension were plated on 2×YT containing agar (2%), kanamycin (50 μg/mL), chloramphenicol (5 μg/mL), dTPT3TP (37.5 μM), dNaMTP (150 μM), and KPi (50 mM, pH 7). Plates were incubated for 14-24 h at 37° C. Colonies were picked and transferred to 500 μL of 2×YT containing kanamycin (50 μg/mL), chloramphenicol (5 μg/mL), dTPT3TP (37.5 μM), dNaMTP (150 μM), KPi (50 mM, pH 7) in a 48-well plate (Ref #677180, Greiner Bio-One). Plates were either refrigerated at 4° C. for ˜12 h followed by incubation at 37° C. at 230 RPM or advanced directly to incubation. After reaching 0.6-1 OD₆₀₀ cultures were sampled as follows: 100 μL was combined with 100 μL glycerol (50%) and frozen at −80° C.; 350 μL was pelleted and frozen for later isolation of genomic DNA; 50 μL was pelleted, washed once with 200 μL diH₂O, pelleted, and resuspended in 200 μL.

The cell suspensions were analyzed by colony biotin-shift PCR (see Supporting Information). Genomic DNA was isolated from saved frozen cell pellets for samples that displayed high colony biotin-shift PCR percent shift values (≥80%) with a PureLink Genomic DNA Mini Kit (Thermo Fisher Scientific) according to manufacturer recommendations. Genomic DNA was analyzed by biotin-shift PCR (see Supporting Information). This analysis revealed high retention clones (Retention_{B}≥90%) for all genetic backgrounds. While these results confirmed successful chromosomal integration of the UBP and remarkably high retention of the UBP in chromosomal DNA, it was suspected that the cells depleted their media of dTPT3TP and dNaMTP during the integration protocol given the protocol's requirement to incubate cells at high cell density. Actively growing cultures of E. coli are known to degrade extracellular dTPT3TP and dNaMTP to their corresponding di- and mono-phosphate and nucleoside species 5. To address this possibility the glycerol stocks of the highest retention samples were used to inoculate 100 μL of 2×YT containing kanamycin (50 μg/mL), chloramphenicol (5 μg/mL), dTPT3TP (37.5 μM), dNaMTP (150 μM), and KPi (50 mM, pH 7) in a 96-well plate. Cultures were grown to approximately 0.6 OD₆₀₀ at 37° C. at 230 RPM. Cells from this culture were plated, picked, grown, and sampled as described above. This “replating” procedure quickly revealed clones for the ΔrecA-Opt and Pol II+ΔrecA-Opt SSOs with undetectable chromosomal UBP loss (Retention_{B}=100%). However despite screening 12 clones for the WT-Opt SSO, no clones with Retention_{B}>91% were discovered. Therefore, we chose to use a WT-Opt integrant (Retention_{B}=91%) that did not undergo the replating procedure for the doubling time and passaging experiments. For ΔrecA-Opt and Pol II⁺ΔrecA-Opt we selected one clone each with Retention_{B}=100% for the doubling time and passaging experiments.

It should be noted that the Pol II⁺ΔrecA strain used in these experiments (FIG. 3B and FIG. 3C) did not have a neo cassette at the former recA locus (P_polB(−)lexA-polB+ΔrecA+FRT+IacZYA::P_lacUV5-ΔΔ(CoOp) col 1.1, Table S1).

### Determination of Strain Doubling Time

Mid-log phase cells WT-Opt, ΔrecA-Opt, and Pol II⁺ΔrecA-Opt SSOs and their corresponding chromosomal UBP integrants (described above) were prepared using the following procedure. Saturated overnight cultures were prepared by inoculation of 2×YT containing chloramphenicol (5 μg/mL), dTPT3TP (37.5 μM), dNaMTP (150 μM), and KPi (50 mM, pH 7) from glycerol stock stabs and overnight growth (approximately 14 h) at 37° C. at 230 RPM. These cells were diluted to 0.03 OD₆₀₀ in 500 μL 2×YT containing chloramphenicol (5 g/mL), dTPT3TP (37.5 μM), dNaMTP (150 μM), and KPi (50 mM, pH 7) and grown at 37° C. at 230 RPM. Growth was monitored by OD₆₀₀. Once cells reached mid-log phase (0.3-0.5 OD₆₀₀), they were diluted to 0.013 OD₆₀₀ in 500 μL 2×YT containing chloramphenicol (5 g/mL), dTPT3TP (37.5 μM), dNaMTP (150 μM), and KPi (50 mM, pH 7) or 2×YT containing chloramphenicol (5 μg/mL) and KPi (50 mM, pH 7) in a 48-well plate and grown at 37° C. at 230 RPM. OD₆₀₀ was measured every 30 min. This procedure was performed in triplicate for each strain starting from inoculation of overnight cultures.

OD₆₀₀ data from each experiment was analyzed to obtain a theoretical cell doubling time (FIG. 3B and Figure S5). OD₆₀₀ measurements corresponding to the exponential growth phase (0.01-0.9) were fit to the following exponential growth model using R version 3.2.4:²⁵

$${OD}_{i} = {OD}_{0} * {2}^{{C}_{Growth} * t}$$

Where UDᵢ is the OD₆₀₀ at time (t), OD₀ is minimum OD₆₀₀ value for a given data set, and C_{Growth} is the growth constant. C_{Growth} was fit using the “nls( )” command. Doubling times (DT) were calculated using the following equation:

$$D T = \frac{1}{{C}_{Growth}}$$

### Passaging of Strains Bearing a Genomic UBP

Glycerol stock stabs of chromosomal UBP integrants from the WT-Opt, ΔrecA-Opt, and Pol II⁺ΔrecA-Opt SSOs (described above) were used to inoculate 500 μL of 2×YT containing kanamycin (50 μg/mL), chloramphenicol (5 μg/mL), dTPT3TP (37.5 μM), dNaMTP (150 μM), and KPi (50 mM, pH 7). Cells were grown to mid log phase (0.5-0.8 OD₆₀₀) at 37° C. at 230 RPM and then diluted to 0.03 OD₆₀₀ in 500 μL of 2×YT containing kanamycin (50 μg/mL), chloramphenicol (5 μg/mL), dTPT3TP (37.5 μM), dNaMTP (150 μM), and KPi (50 mM, pH 7) in a 48-well plate and grown at 37° C. at 230 RPM. The cultures inoculated at 0.03 OD₆₀₀ were considered the starting point (Doublings=0) for passaging. The cultures were grown to 1-1.5 OD₆₀₀ corresponding to approximately 5 cell doublings. This growth from 0.03 to 1-1.5 OD₆₀₀ was considered one “passage” with one passage corresponding to approximately 5 cell doublings. After these samples reached 1-1.5 OD₆₀₀, another passage was started by diluting cells to 0.03 OD₆₀₀ in fresh media of the same composition. After dilution, the 1-1.5 OD₆₀₀ culture was sampled as follows: 100 μL was combined with 100 μL glycerol (50%) and frozen at −80° C.; 350 μL was pelleted and frozen for later isolation of genomic DNA; and 50 μL was pelleted, washed once with 200 μL diH₂O, pelleted, and resuspended in 200 μL. The passaging process was repeated for a total of 15 passages, corresponding to approximately 80 cell doublings for all three strains.

Throughout passaging, colony biotin-shift PCR analysis (see Supporting Information) was performed on the cell suspension samples. This revealed that retention had declined to <10% in WT-Opt after 15 passages. Therefore, this strain was no longer passaged. In contrast, retention remained at 60-80% in ΔrecA-Opt and Pol II⁺ΔrecA-Opt. Therefore, an additional passage was performed as above for these strains. Retention remained unchanged now a total 16 passages. Therefore, these strains were subjected to 4 additional passages at a higher dilution factor that corresponded to approximately 13 cell doublings per passage (growth from approximately 0.0001 to 1-1.5 OD₆₀₀). At this point ΔrecA-Opt and Pol II⁺ΔrecA-Opt integrants had experience approximately 130 cell doublings and UBP retention remained>40% according to colony biotin-shift PCR analysis. Further passaging was deemed unnecessary and the experiment was stopped for more rigorous analysis of the genomic DNA samples gathered during passaging. This experiment was performed in triplicate starting from inoculation of media with the genomic integrant glycerol stock stabs.

After completing the passaging experiment, genomic DNA was isolated and analyzed by biotin-shift PCR (FIG. 3C) (see Supporting Information). The slow, then rapid loss of the UBP in WT-Opt suggested that multiple processes contributed to UBP loss. It was suspected that the P_{lacUV5}-PtNTT2(66-575) may have been mutated during the experiment, as expression of PtNTT2 causes a slight growth defect.³ Thus, cells that inactivate the transporter through mutation gain a fitness advantage and can rapidly dominate the experimental population. This hypothesis was explored through isolation of individual clones from the end of WT-Opt passaging and PCR analysis of purified genomic DNA (see Supporting Information and Figure S7). Primer walking for several clones revealed that all genes between cat and insB-4 including PtNTT2(66-575) had been deleted in these cells. The insB-4 gene encodes one of two proteins required for the transposition of the IS1 transposon.²⁶ Sequencing of one clone confirmed that IS1 inserted at PtNTT2(66-575)(T1495) corresponding to a 15890 base pair deletion.

After confirmation of the PtNTT2(66-575) mutation event, the emergence of deletion mutants was assessed by PCR analysis of genomic DNA samples from WT-Opt integrant passaging (see Supporting Information and Figure S7B). This analysis revealed that several amplicons of sizes corresponding to IS1-mediated PtNTT2(66-575) deletion events appear in passaging samples during the rapid phase of UBP loss.

It was also observed that one replicate of the Pol II⁺ΔrecA-Opt integrant rapidly lost the UBP at the same time as the WT-Opt integrants, strongly suggesting that this replicate may have been contaminated with WT-Opt cells during the passaging. This possibility was confirmed using colony PCR analysis, which revealed that this replicate became contaminated with WT-Opt cells at passages corresponding rapid loss of the UBP (see Supporting Information and Figure S6). Therefore, data from this replicate was only used from samples without WT-Opt cell contamination.

### Bacterial Strains and Plasmids

All strains used in this study (Table S1; provided as a separate Supporting file) were constructed from E. coli-BL21(DE3) through lambda red recombineering unless otherwise indicated. Gene knock-out cassettes were obtained through PCR amplification (using either OneTaq or Q5 according to manufacturer recommendations (New England Biolabs)) of either genomic DNA of Keio collection strains or pKD13 with the relevant primers (Table S1). Functional genetic knock-in cassettes, polA(D424A,K890R) and Pol II+ (FIG. S4) were constructed through overlapping PCR. Strains were made competent for dXTP import through either transformation with pACS2 or pACS2-dnaQ(D12N) or integration of the PlacUV5⁻PtNTT2(66-575)+cat cassette at the lacZYA locus (Figure S1). The construction of pACS2 and P_{lacUV5}-PtNTT2(66-575)+cat has been described previously.³ pACS2-dnaQ(D12N) was constructed through Gibson assembly of PCR amplicons. PtNTT2 function was confirmed in every relevant strain using a radioactive dATP uptake assay.

### Exonuclease Deficient Pol I and III.

DNA Pol I and III are conditionally essential and essential genes, respectively. Therefore, unlike the SOS-regulated polymerases they could not be examined by genetic knockout. Instead we constructed 3′-5′ exonuclease deficient mutants for these enzymes. Pol I (polA) was made 3′-5′ exonuclease deficient by mutating the active site of its exonuclease domain (D424A). This was accomplished through two phases of lambda red recombineering (Figure S4). First polA was truncated to its 5′-3′ exonuclease domain (removing both polymerase and 3′-5′ exonuclease domains). Second polymerase and 3′-5′ exonuclease domains were reintroduced with the D424A mutation. Due to the length of the gene, PCR mutations were generated in the amplicon used for integrations. This resulted in the K890R mutation. However, since K890 is a surface exposed residue on a disordered loop of the protein its mutation to arginine was expected to have minimal effect on protein function. Moreover, a lysine to arginine maintains approximate charge and size of the residue.

DNA Pol III holoenzyme is a multi-enzyme complex with separate polymerase and 3′-5′ exonuclease enzymes. The exonuclease enzyme (dnaQ) is thought to play a structural role in the Pol III holoenzyme in addition to its editing activity. Therefore, deletion of dnaQ removes Pol III editing activity but also prevents cell growth unless compensatory mutations are added to other parts of the holoenzyme. Therefore, we chose to examine Pol III's role in UBP replication through expression of a mutator dnaQ mutant (D12N) from the plasmid, pACS2+dnaQ(D12N) (Figure S1). The expression of dnaQ(D12N) from a multicopy plasmid has been previously demonstrated to produce a dominant mutator phenotype in E. coli despite the expression of wild-type DnaQ from the chromosomal copy of the gene. pACS2+dnaQ(D12N) expresses dnaQ(D12N) with both of the native gene promoters.

Fitness Costs from Genetic Optimization of the SSO.

Deletion of recA clearly leads to greatly improved retention of the UBP in many sequences. While this is highly desirable, recA deletion does carry some fitness costs. Strains deficient in recA are known to have lower tolerance for DNA damage. However given that all near-term applications of the SSO will take place in highly controlled environments, we do not expect this to be problematic. Additionally, recA deletion increases the doubling time as measured in FIG. S5. However, these experiments were performed primarily to illustrate the difference in growth rate for strains bearing a chromosomal UBP growing in the presence or absence of dNaMTP and dTPT3TP. Several factors complicate directly relating strain fitness to the measured doubling times. The main complication is that cells in solution can increase OD600 by altering their morphology rather than actually increasing cell number. Regardless, the measured doubling time for L recA-Opt (˜18 min longer than WT-Opt) suggests that deletion of recA leads to a significantly reduced growth rate. However given the benefits of this modification, this reduced growth rate is an acceptable trade-off. It should also be noted that some data points in FIG. 8 are difficult to rationalize. For example, the presence of the chromosomal UBP appears to decrease the doubling time in L recA-Opt and Pol II⁺μL recA-Opt.

### Biotin-Shift Analysis

Retention of UBPs in pINFs and chromosomal DNA was measured as described previously with the following modifications. All biotin-shift PCRs were run in a 15-iiL volume with primers (1 iiM, Table S1), d5SICSTP (65 iiM), dMMO2bioTP (65 μM), dNTPs (400 iiM), MgSO₄ (2.2 mM), OneTaq DNA Polymerase (0.018 U/iiL), DeepVent DNA Polymerase (0.007 U/iiL, New England Biolabs), SYBR Green I (1×, Thermo Fisher Scientific) and OneTaq Standard Reaction Buffer (1×). The amount of sample DNA added to a biotin-shift PCR and the temperature regime varied depending on the nature of the sample.

For in vivo replicated pINFs (FIG. 1) from the genetic knockout experiments, the exact concentration of pINF DNA could not be determined as pINF and pACS2 DNA were both captured by the ZR Plasmid Miniprep-Classic kit. Therefore, 0.5 tiL (approximately 3 ng total DNA) of the purified sample was used as template DNA for biotin shift PCR. Reactions were subjected to the following temperature regime (time in mm:ss) in a CFX Connect Real-Time PCR Detection System (BioRad): [96° C. 02:00|12-18×(96° C. 00:15|48° C. 00:15|68° C. 04:00)]. Reaction progression was monitored by SYBR Green I fluorescence and reactions were stopped and stored at 4° C. at the end of the 68° C. step just after the reaction exited the exponential phase of PCR (typically 17 cycles). For clonal pINF experiments (FIG. 3A), 3 ng of purified plasmid DNA was used as template DNA for biotin-shift PCR analysis. Reactions were subjected to the same temperature regime and monitoring as above.

Golden Gate constructed pINFs and UBP containing oligonucleotides were amplified as described above using 1 ng and 7.5 μg, respectively, of sample as template DNA for the biotin-shift PCR.

For the chromosomal UBP experiments, both colony biotin shift PCR and biotin shift PCR amplification of purified genomic DNA was performed. For colony biotin shift PCR, 2 tiL of cell suspension (see above) was added to biotin shift PCR as template. Reactions were subjected to the following temperature regime and monitored as above (time in mm:ss): [96° C. 02:00|5×(96° C. 00:15|60° C. 00:15|68° C. 04:00)|20-22×(96° C. 00:15|48° C. 00:15|68° C. 04:00)]. For biotin shift PCR amplification of purified genomic DNA, 30-125 ng of purified genomic DNA was used as template for biotin shift PCR. These reactions were subjected to the following temperature regime and monitored as above (time in mm:ss): [96° C. 02:00|5× (96° C. 00:15|60° C. 00:15|68° C. 04:00)|10-16× (96° C. 00:15|48° C. 00:15|68° C. 04:00)].

The percentage of biotin labeled amplicons from any of the biotin shift PCRs described above was determined by mixing 1 tiL of biotin-shift PCR with 2.5 tiL of streptavidin (2 tig/μL, Promega) and 1 tiL of Purple Loading Dye (6×, New England Biolabs) and resolving streptavidin-DNA complexes on a native 6% polyacrylamide gel. Gels were stained in 50 mL TBE with 1×SYBR Gold (Thermo Fisher Scientific). Gels were imaged on a Molecular Imager Gel Doc XR System (Bio-Rad, Hercules, CA) with a 520DF30 62 mm (Bio-Rad) filter. Image exposure time was limited to prevent saturation of the CCD for pixels in the bands of interests. The fluorescence intensity of DNA and streptavidin-DNA bands was quantified using Quantity One software v4.6.9 (Bio-Rad) using local background subtraction. The percent shift (S) was then calculated for each sample based on the pixel volume of the DNA (VDNA) and streptavidin-DNA(VSA-DNA) bands.

Quantification of UBP retention by biotin-shift PCR analysis requires normalization of sample shift values to the shift value of an in vitro constructed UBP containing DNA. For the in vivo replication experiments in genetic knockouts (FIG. 1) sample percent shift values (S_{S}) were normalized to the percent shift of the Golden Gate assembled pINF_{(SGG)} used to initiate the in vivo replication experiment (Retention_{A}).

While dTPT3-dNaM and related analogs have been demonstrated to be replicated in PCR with high fidelity¹⁰ and with limited sequence context bias,¹¹ the UBP is mutated with some frequency in PCR. Therefore, pINF production by Golden Gate assembly of PCR-generated UBP containing DNA results in the production of some mutant, fully natural pINFs. Given that the experiments presented in FIG. 1 did not isolate individual pINF transformants, these fully natural pINFs will be transformed into experimental cells and replicated alongside UBP-containing pINFs. This places an upper bound on the theoretical maximum percent shift value for a given sample. If no UBP loss occurs in vivo, the percent shift of an in vivo sample will equal that of the Golden Gate assembled pINF used to initiate the experiment (Retention_{A}=100%). Some replicates (particularly in the ΔrecA backgrounds) displayed over 100% Retention_{A}. This likely results from sampling of the Golden Gate assembled pINFs. If the transformation happens to result in transformation of only UBP containing plasmids and the cells replicate these plasmids with no UBP loss, normalization of S_{S} to _{SGG} will result in a value greater than 100%. If UBP replication occurs with perfect fidelity, the same logic applies to any sampling of the Golden Gate assembled pINFs that results in a higher percentage of UBP containing pINFs in the cells than the Golden Gate products. Thus, this is most apparent in the ΔrecA strains due to their high UBP retention.

The clonal pINF and chromosomal UBP experiments (FIG. 3) did examine individual pINF transformants. Therefore, PCR-mutated fully natural pINFs can produce individual clones that display no UBP content (S_{S}=0%), but they do not impact the retention of other clones that received a UBP-containing pINF. Thus, SSOs that receive and faithfully replicate a UBP-containing pINF can be expected to have shift values that match the shift value of biotin-shift PCR analysis of a chemically synthesized UBP-containing oligonucleotide (assumed to have 100% UBP content). To convert sample shift values to retention values (Retention_{B}) for the experiments shown in FIG. 3, sample shift values (S_{S}) were normalized to the shift values of chemically synthesized UBP-containing oligonucleotides (S_{O}). If no UBP loss occurs in vivo, the percent shift of the in vivo sample will equal that of the chemically synthesized UBP containing oligonucleotide (Retention_{B}=100%).

Outlier and Low Golden Gate Assembled pINF Removal

All data gathered is presented with the following exceptions. One outlier (by Grubbs test) was removed from FIG. 1B. This data point showed no retention for the ΔuvrC mutant replicating the TCAXAGT sequence. As described above, UBP retention in the Golden Gate assembled pINFs used to initiate FIG. 1 experiments places an upper bound on retention achievable after in vivo replication. Some Golden Gate assembled pINFs preparations showed very low retention. In vivo replication data gathered with these constructs is not presented. Specifically, data was discarded if the Golden Gate assembled pINF's % Shift values were below the following cut-offs for each sequence: GTAXAGA-60%, TCAXAGT-70%, TCGXGGT-55%, TCTXGGT-50%, TCCXCGT-55%, and TCCXGGT-55%.

### Example 3. Knockout of IS1 Increases Stability of Nucleoside Triphosphate Transporter PtNTT2

To assess whether deflection of the transposable element, IS1, contributed to increased stability (evidenced by activity) of the nucleoside triphosphate transporter, PtNTT2, during long-term growth, YZ3 and ΔIS1 were serially passaged in triplicate on 2×YT+50 mM KPi+5 ug/mL Chlor (Media) and assayed for PtNTT2 activity (Rad/OD600). Passage 1 was started through inoculation of 700 uL of Media with a glycerol stock stab. Cultures were grown overnight (˜15 hrs) to saturation. Cells were diluted 350-fold into 700 uL of fresh media. This was repeated for 6 total passages. Cells were then passaged 4 additional times with the dilution factor increased to 350,000-fold. Assessment of passaged populations PtNTT2 activity demonstrates that the ΔIS1 strain maintains greater PtNTT2 activity during long-term growth, presumably through elimination of the IS1-mediated PtNTT2 deletion pathway. Since the deletion of PtNTT2 contributed to the loss of unnatural base pairs (UBPs), these results suggest that an engineered host cell or semi-synthetic organism lacking in the IS1-mediated PtNTT2 deletion pathway will show increased retention of unnatural base pairs, and thus an increase in the production of polypeptides comprising unnatural amino acids, as well as the nucleic acid molecules encoding them.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the invention and that methods and structures within the scope of these claims and their equivalents be covered thereby.

What is claimed is:

1. An engineered host cell comprising:
a. a first nucleic acid molecule comprising an unnatural nucleotide; and
b. optionally, a second nucleic acid molecule encoding a modified transposition-associated protein or transposable element.

2. The engineered host cell of claim 1, further comprising:
a. a third nucleic acid molecule encoding a modified nucleoside triphosphate transporter, wherein the third nucleic acid molecule is incorporated in a genomic sequence of the engineer host cell, or comprises a plasmid encoding the modified nucleoside triphosphate transporter.

3. The engineered host cell of claim 1, wherein the modified nucleoside triphosphate transporter exhibits increased stability of expression in the engineered host cell as compared to an expression in an equivalent engineered host cell that does not comprise the second nucleic acid molecule encoding the modified transposition-associated protein.

4. The engineered host cell of claim 2, wherein the modified nucleoside triphosphate transporter comprises a deletion of an entire nucleic acid molecule encoding the nucleoside triphosphate transporter, an N-terminal truncation, a C-terminal truncation, or a truncation of both termini.

5. The engineered host cell of claim 2, wherein modified nucleoside triphosphate transporter comprises a nucleoside triphosphate transporter from Phaeodactylum tricornutum (PtNTT₂).

6. The engineered host cell of claim 5, wherein the PtNTT₂ is under the control of a promoter selected from a pSC plasmid or a promoter from a lac operon.

7. The engineered host cell of claim 2, further comprising:
a. a Cas9 polypeptide or variants thereof, and
b. a single guide RNA (sgRNA) comprising a crRNA-tracrRNA scaffold, wherein the combination of Cas9 polypeptide or variants thereof and sgRNA modulates replication of the first nucleic acid molecule encoding the unnatural nucleotide.

8. The engineered host cell of claim 1, wherein the second nucleic acid molecule comprises a gene comprising catalase (cat), IS1 protein insB-4 (insB-4), IS1 protein insA-4 (insA-4), or a combination thereof.

9. The engineered host cell of claim 1, wherein the modified transposition-associated protein comprises Insertion element IS1 4 protein InsB, Insertion element IS1 4 protein InsA, or a combination thereof, and wherein the modified transposable element comprises IS1.

10. The engineered host cell of claim 8, wherein the gene comprises one or more deletions, wherein the one or more deletions comprise an N-terminal deletion, a C-terminal deletion, a truncation at both termini, an internal deletion, and/or a deletion of the entire gene.

11. The engineered host cell of claim 1, further comprising a fifth nucleic acid molecule encoding a modified DNA repair response-associated protein, wherein the DNA repair response comprises recombinational repair, SOS response, nucleotide excision repair, or methyl-directed mismatch repair, or a combination thereof.

12. The engineered host cell of claim 11, wherein the modified DNA repair response-associated protein comprises RecA, Rad51, RadA, or LexA, or a combination thereof.

13. The engineered host cell of claim 1, wherein the engineered host cell is a prokaryotic cell comprising an Escherichia coli cell, Escherichia coli BL21 (DE3) cell.

14. The engineered host cell of claim 1, wherein the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N₄-ethylcytosine, N-2 substituted purines, N-6 substituted purines, O—6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N₆-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N₆-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(β-amino-3-N-2-carboxypropyl) uracil, (acp3)w, and 2,6-diaminopurine and those in which the purine or pyrimidine base is replaced with a heterocycle.

15. The engineered host cell of claim 1, wherein the unnatural base is selected from the group consisting of:

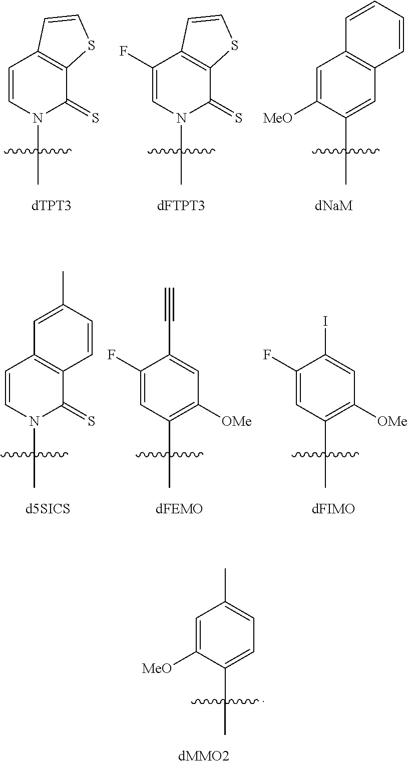

16. The engineered host cell of claim 1, wherein the unnatural nucleotide further comprises an unnatural sugar moiety.

17. The engineered host cell of claim 16, wherein the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)ₙOCH₃, —O(CH₂)n NH₂, —O(CH₂)n CH₃, —O(CH₂)n—ONH₂, and —O(CH₂)ₙON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

18. A method of increasing the production of a nucleic acid molecule comprising an unnatural nucleotide, comprising:
a) incubating an engineered host cell with a plurality of unnatural nucleotides, wherein the engineered host cell comprises a modified nucleoside triphosphate transporter and optionally, a modified transposition-associated protein or transposable element; and
b) incorporating the plurality of unnatural nucleotides into one or more newly synthesized DNA strands, thereby generating the unnatural nucleic acid molecule;
wherein the modified transposition-associated protein or transposable element and the modified nucleoside triphosphate transporter increases retention of unnatural base pairs comprising the unnatural nucleotides in the one or more newly synthesized DNA strands.

19. The method of claim 18, wherein the modified transposition-associated protein comprises Insertion element IS1 4 protein InsB, Insertion element IS1 4 protein InsA, or a combination thereof, and wherein the modified transposable element comprises IS1.

20. The method of claim 18, wherein the modified nucleoside triphosphate transporter comprises a codon optimized nucleoside triphosphate transporter from Phaeodactylum tricornutum (PtNTT₂).

21. The method of claim 18, wherein the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N₄-ethylcytosine, N-2 substituted purines, N-6 substituted purines, 0-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N₆-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N₆-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(β-amino-3-N-2-carboxypropyl) uracil, (acp3)w, and 2,6-diaminopurine and those in which the purine or pyrimidine base is replaced with a heterocycle.

22. The method of claim 19, wherein the unnatural base is selected from the group consisting of:

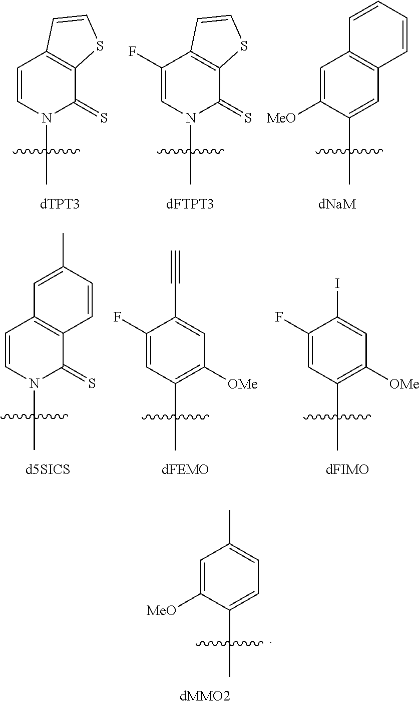

23. The method of claim 19, wherein the unnatural nucleotide further comprises an unnatural sugar moiety.

24. The method of claim 23, wherein the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)ₙOCH₃, —O(CH₂)n NH₂, —O(CH₂)n CH₃, —O(CH₂)n—ONH₂, and —O(CH₂)ₙON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkyl amino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

25. A method of preparing a modified polypeptide comprising an unnatural amino acid, comprising:
c) incubating an engineered host cell with a plurality of unnatural nucleotides, wherein the engineered host cell comprises a modified nucleoside triphosphate transporter and, optionally, a modified transposition-associated protein or transposable element; and
d) incorporating the plurality of unnatural nucleotides into one or more newly synthesized DNA strands, thereby generating the unnatural nucleic acid molecule;
wherein the modified transposition-associated protein or transposable element and the modified nucleoside triphosphate transporter increases retention of unnatural base pairs which facilitates incorporation of the plurality of unnatural nucleotides into the newly synthesized polypeptide to generate the modified polypeptide.

26. The method of claim 25, wherein the modified transposition-associated protein comprises Insertion element IS1 4 protein InsB, Insertion element IS1 4 protein InsA, or a combination thereof, and wherein the modified transposable element comprises IS1.

27. The method of claim 25, wherein the modified nucleoside triphosphate transporter comprises a codon optimized nucleoside triphosphate transporter from Phaeodactylum tricornutum (PtNTT₂).

28. The method of claim 25, wherein the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifluoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N₄-ethylcytosine, N-2 substituted purines, N-6 substituted purines, O—6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N₆-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N₆-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(β-amino-3-N-2-carboxypropyl) uracil, (acp3)w, and 2,6-diaminopurine and those in which the purine or pyrimidine base is replaced with a heterocycle.

29. The method of claim 25, wherein the unnatural base is selected from the group consisting of:

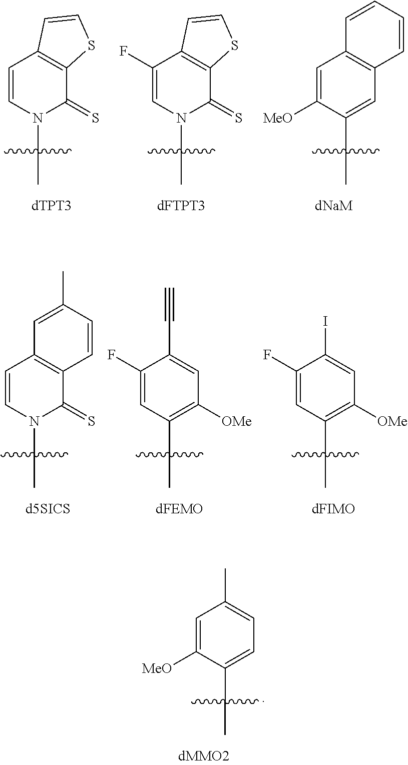

30. The method of claim 25, wherein the unnatural nucleotide further comprises an unnatural sugar moiety selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)n O]mCH₃, —O(CH₂)ₙOCH₃, —O(CH₂)n NH₂, —O(CH₂)n CH₃, —O(CH₂)n—ONH₂, and —O(CH₂)ₙON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.


SEQUENCE LISTING (32 sequences)

SEQ ID NO: 1 - DNA, 2540 bp, Escherichia coli
  source 1..2540: mol_type = genomic DNA
aaaccaggctcccccaagcctggttgtaggctggagctgcttcgaagttcctatactttctagagaataggaacttcgaactgcaggtcgacggatccccggaatttttttataaatacacagccaatcaaacgaaaccaggctataatcaagcctggttttttgatggaattacagcgtggcgcaggcaggttttatcttaacccgacactggcgggacaccccgcaagggacagaagtctccttctggctggcgacggacaacgggccgttgcaggttacgcttgcaccgcaagagtccgtggcgtttattcccgccgatcaggttccccgcgctcagcatattttgcagggtgaacaaggcgttcgcctgacaccgctggcgttaaaggattttcaccgccagccggtgtatggcctttactgtcgcgcccatcgccaattgatgaattacgaaaagcgcctgcgtgaaggtggcgttaccgtctacgaggccgatgtgcgcccgccagaacgctatctgatggagcggtttatcacctcaccggtgtgggtcgagggtgatatgcacaatggcgctatcgttaatgcccgtctgaaaccgcatcccgactatcgtccgccgctcaagtgggtttctattgatattgaaaccacccgccacggtgagctgtactgcatcggcctggaaggctgcgggcagcgcatcgtttatatgctggggccagagaatggcgacgcctccgcgctcgattttgaactggaatacgtcgccagccgcccgcagctactggaaaaactcaacgcctggtttgccaactacgatcctgatgtgatcatcggttggaacgtggtgcagttcgatctgcgaatgctgcaaaaacatgccgagcgttaccgtattccgctgcggctggggcgcgataacagtgagctggagtggcgcgagcacggctttaaaaacggcgtcttttttgcccaggctaaaggtcggctaattatcgacggtatcgaggcgctgaaatccgcgttctggaatttctcttcattctcgctggaaaccgtcgctcaggagttattaggcgaaggaaaatctatcgataacccgtgggatcgaatggacgaaattgaccgccgtttcgccgaagataaacctgcgctggcgacttataacctgaaagattgcgagctggtgacgcagattttccataaaactgaaatcatgccatttttactcgaacgagcgacggtgaacggcctgccggtagaccgccacgggggatcggtggcggcgtttggtcatctctattttcctcgtatgcaccgcgctggttatgtcgcgcctaatctcggcgaagtgccgccacacgccagccctggcggctacgtgatggattcacggccagggctttatgattcggtactggtgctggactataaaagcctgtacccgtcgatcatccgcacctttctgattgatcccgtcgggctggtggaaggcatggcgcagcctgatccagagcacagtaccgaaggttttctcgatgcctggttctcgcgagaaaaacattgcctgccggagattgtgactaacatctggcacgggcgcgatgaagccaaacgccagggtaacaaaccgctgtcgcaggcgctgaaaatcatcatgaatgccttttatggcgtgctcggcaccaccgcctgccgcttcttcgatccgcggctggcatcgtcgatcaccatgcgtggtcatcagatcatgcggcaaaccaaagcgttgattgaagcacagggctacgacgttatctacggcgataccgactcaacgtttgtctggctgaaaggcgcacattcggaagaagaagcggcgaaaatcggtcgtgcactggtgcagcacgttaacgcctggtgggcggaaacgctgcaaaaacaacggctgaccagcgcattagaactggagtatgaaacccatttctgccgttttctgatgccaaccattcgcggagccgataccggcagtaaaaagcgttatgccggactgattcaggagggcgacaagcagcggatggtgtttaaagggctggaaaccgtgcgcaccgactggacgccgctggcccagcagtttcagcaggagctatacctgcgcatcttccgcaacgagccatatcaggaatatgtacgcgaaaccatcgacaaactgatggcgggtgaactggatgcgcgactggtttaccgtaaacgccttcgccgtccgctgagcgagtatcagcgtaatgtgccgcctcatgtacgcgccgctcgccttgccgatgaagaaaaccaaaagcgtggtcgccccttgcaatatcagaatcgcggcaccattaagtacgtatggaccaccaacggcccggagccgctggactaccaacgttcaccactggattacgaacactatctgacccgccagctacaacccgtggcggagggaatactcccttttattgaggataattttgctacacttatgaccgggcaacttgggctattttgagcaaaaaaaa

SEQ ID NO: 2 - DNA, 2436 bp, Escherichia coli
  source 1..2436: mol_type = genomic DNA
tgactgtataaaaccacagccaatcaaacgaaaccaggctataatcaagcctggttttttgatggaattacagcgtggcgcaggcaggttttatcttaacccgacactggcgggacaccccgcaagggacagaagtctccttctggctggcgacggacaacgggccgttgcaggttacgcttgcaccgcaagagtccgtggcgtttattcccgccgatcaggttccccgcgctcagcatattttgcagggtgaacaaggcgttcgcctgacaccgctggcgttaaaggattttcaccgccagccggtgtatggcctttactgtcgcgcccatcgccaattgatgaattacgaaaagcgcctgcgtgaaggtggcgttaccgtctacgaggccgatgtgcgcccgccagaacgctatctgatggagcggtttatcacctcaccggtgtgggtcgagggtgatatgcacaatggcgctatcgttaatgcccgtctgaaaccgcatcccgactatcgtccgccgctcaagtgggtttctattgatattgaaaccacccgccacggtgagctgtactgcatcggcctggaaggctgcgggcagcgcatcgtttatatgctggggccagagaatggcgacgcctccgcgctcgattttgaactggaatacgtcgccagccgcccgcagctactggaaaaactcaacgcctggtttgccaactacgatcctgatgtgatcatcggttggaacgtggtgcagttcgatctgcgaatgctgcaaaaacatgccgagcgttaccgtattccgctgcggctggggcgcgataacagtgagctggagtggcgcgagcacggctttaaaaacggcgtcttttttgcccaggctaaaggtcggctaattatcgacggtatcgaggcgctgaaatccgcgttctggaatttctcttcattctcgctggaaaccgtcgctcaggagttattaggcgaaggaaaatctatcgataacccgtgggatcgaatggacgaaattgaccgccgtttcgccgaagataaacctgcgctggcgacttataacctgaaagattgcgagctggtgacgcagattttccataaaactgaaatcatgccatttttactcgaacgagcgacggtgaacggcctgccggtagaccgccacgggggatcggtggcggcgtttggtcatctctattttcctcgtatgcaccgcgctggttatgtcgcgcctaatctcggcgaagtgccgccacacgccagccctggcggctacgtgatggattcacggccagggctttatgattcggtactggtgctggactataaaagcctgtacccgtcgatcatccgcacctttctgattgatcccgtcgggctggtggaaggcatggcgcagcctgatccagagcacagtaccgaaggttttctcgatgcctggttctcgcgagaaaaacattgcctgccggagattgtgactaacatctggcacgggcgcgatgaagccaaacgccagggtaacaaaccgctgtcgcaggcgctgaaaatcatcatgaatgccttttatggcgtgctcggcaccaccgcctgccgcttcttcgatccgcggctggcatcgtcgatcaccatgcgtggtcatcagatcatgcggcaaaccaaagcgttgattgaagcacagggctacgacgttatctacggcgataccgactcaacgtttgtctggctgaaaggcgcacattcggaagaagaagcggcgaaaatcggtcgtgcactggtgcagcacgttaacgcctggtgggcggaaacgctgcaaaaacaacggctgaccagcgcattagaactggagtatgaaacccatttctgccgttttctgatgccaaccattcgcggagccgataccggcagtaaaaagcgttatgccggactgattcaggagggcgacaagcagcggatggtgtttaaagggctggaaaccgtgcgcaccgactggacgccgctggcccagcagtttcagcaggagctatacctgcgcatcttccgcaacgagccatatcaggaatatgtacgcgaaaccatcgacaaactgatggcgggtgaactggatgcgcgactggtttaccgtaaacgccttcgccgtccgctgagcgagtatcagcgtaatgtgccgcctcatgtacgcgccgctcgccttgccgatgaagaaaaccaaaagcgtggtcgccccttgcaatatcagaatcgcggcaccattaagtacgtatggaccaccaacggcccggagccgctggactaccaacgttcaccactggattacgaacactatctgacccgccagctacaacccgtggcggagggaatactcccttttattgaggataattttgctacacttatgaccgggcaacttgggctattttgagcaaaaaaaa

SEQ ID NO: 3 - DNA, 2008 bp, Escherichia coli
  source 1..2008: mol_type = genomic DNA
ttattgcacgaagcgcgaggtaacgtactggctgtaatcattcgctacagccgggaccttgccctgctctttcaaaaactgcgcggtgtcgatgatcgctttgttcaccggtccggtcagttctgccgtttgttgctgcggcgtcagataggtattccccttcaccagccccggaacgtcaccttcaggcacgccgcttaaccgcgccagtttgctgatgttttccggctgtttcagccacgcgtctgggttagcaatgtacggttgctgagcatcgatggcgcttttggcgaacgctttcaccacctcaggatgtttctcggcaaaatctttgcgcaccacccagacgtccagcgttggcgcaccccactgcccgacctgttcagaatcggtcagcactttgccgtctttttccagggcgttaaccgccggtgcccagacataagcaccatcaatatctccccgctgccatgcagcgataatcgcgggcggctgcaggttcacaatctccacttgcccaggtttaatgccccagtgtttcagcgctgccagcaggctgtagtgggtggtagagataaacggtacggcgatgcgtttgccaatcagatcttccggtttgctgatagttttctttaccaccagcgcttctgagttacccagttttgacgccagcaagaagacttcaatcggcacctgttggctggctgcaaccgctaacgggctggaaccaaggttgccgatttgcacgtcgccagaagccagcgcccgcacgatgctggctccgctgtcaaacttacgccagtccacggttgctccgctttctttagcaaaggtgttgtcggcctgagccactttcgccggttcggctgaggtttgatacgccacggtgacgttcaccgcctgtgcctgaaaagcgatgaatgccagtgcggcaagaagtgtgtttcgcgatgaaattgccatgattgtctgctcccctgtcttgttatgggagcagtattcaggaataaaaacattcattaaaagaattagtcgttatcgcacagatgattttattcttagcaaaaaaacggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaactgcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacaggctccggaagacggttgttgcgcacgtattcggtgaacgcactatggcgacgctggggcgtcttatgagcctgatgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattaccaggccgacaaaatggaggcaaagaccaacaaagaaaagtagttaacctaggctgctgccaccgctgagcaataagactcctgttgatagatccagtaatgacctcagaactccatctggatttgttcagaacgctcggttgccgccgggcgttttttattggtgagaat

SEQ ID NO: 4 - DNA, 17210 bp, Escherichia coli
  source 1..17210: mol_type = genomic DNA
ttattgcacgaagcgcgaggtaacgtactggctgtaatcattcgctacagccgggaccttgccctgctctttcaaaaactgcgcggtgtcgatgatcgctttgttcaccggtccggtcagttctgccgtttgttgctgcggcgtcagataggtattccccttcaccagccccggaacgtcaccttcaggcacgccgcttaaccgcgccagtttgctgatgttttccggctgtttcagccacgcgtctgggttagcaatgtacggttgctgagcatcgatggcgcttttggcgaacgctttcaccacctcaggatgtttctcggcaaaatctttgcgcaccacccagacgtccagcgttggcgcaccccactgcccgacctgttcagaatcggtcagcactttgccgtctttttccagggcgttaaccgccggtgcccagacataagcaccatcaatatctccccgctgccatgcagcgataatcgcgggcggctgcaggttcacaatctccacttgcccaggtttaatgccccagtgtttcagcgctgccagcaggctgtagtgggtggtagagataaacggtacggcgatgcgtttgccaatcagatcttccggtttgctgatagttttctttaccaccagcgcttctgagttacccagttttgacgccagcaagaagacttcaatcggcacctgttggctggctgcaaccgctaacgggctggaaccaaggttgccgatttgcacgtcgccagaagccagcgcccgcacgatgctggctccgctgtcaaacttacgccagtccacggttgctccgctttctttagcaaaggtgttgtcggcctgagccactttcgccggttcggctgaggtttgatacgccacggtgacgttcaccgcctgtgcctgaaaagcgatgaatgccagtgcggcaagaagtgtgtttcgcgatgaaattgccatgattgtctgctcccctgtcttgttatgggagcagtattcaggaataaaaacattcattaaaagaattagtcgttatcgcacagatgattttattcttagcaaaaaaactgtaggctggagctgcttgaagttcctatactttctagagaataggaacttcgaactgcaggtcgacggatccccggaattagaacgtaatttacctgccggaacttattcactccgacaagaacttatccgtacaggagattaaaatgataaaacggacgttattagcggcggccatttttagcgcattgcccgcttatgccgggttaacttccattaccgcgggctacgattttaccgattattctggcgatcatggcaaccgtaatttagcgtatgctgaactggtggcgaaagttgaaaacgcaacgctgctttttaatctttcccaggggcgtcgtgattatgaaactgaacatttcaatgccactcgcggtcagggtgcggtctggtataaatggaataactggctgacaacccgaacgggtattgcctttgcggataatacgccggtctttgcccgccaggattttcgtcaggatattaacctggccctgttgccaaaaacgcttttcacgaccggttatcgctacactaaatattacgatgatgtcgaagtcgatgcctggcaaggcggcgtatcactctatactggcccggtgatcaccagctaccgctatacccattatgactccagcgatgcaggtggtagttatagcaatatgatttccgtgcgtctgaatgacccgcgcggcactggttatacgcaactatggctaagccgcggaacaggcgcttacacctatgactggacgccagaaacacgctacggcagcatgaagagcgtcagtctgcaacgtattcaaccgctgactgagcaacttaatctcgggctgacggcaggtaaagtgtggtacgacaccccaaccgatgattataacggtctgcaacttgcagcccatctgacctggaaattctgattccttctgccgcccgctatccggggcggccttccctgccgattagcccccccctttcctctttgttttccgaccacattcaccggataaattttattctccagtgttatatactataggggggtatgcattgacatatagcatacccccctatagtatattgcgtgcagataatgaggtgcgaaatgcccagtactccggaagagaagaaaaaggtccttactcgagttcgtcgtattcgagggcagattgatgctctggaacggtcgctggagggtgatgccgaatgccgtgccatactccaacagatcgctgccgttcggggcgcggctaatgggctgatggcagaagtgcttgaaagccatatccgggaaacgtttgaccgaaatgactgctacagccgcgaagtcagccaatccgttgacgacactattgaactggttcgagcctatcttaaatagctgaatctattaccatattgaggaagagcgagagatgaaatcacgtgctgccgttgcatttgctcccggtaaaccgctggaaatcgttgaaattgacgttgcaccaccgaaaaaaggtgaagtgctgattaaagtcacccataccggcgtttgccataccgacgcatttaccctctccggggatgacccggaaggtgtattcccggtggttctcggtcacgaaggggccggcgttgtggttgaagtcggtgaaggcgtaaccagcgtcaaacctggcgaccatgtgatcccgctttacaccgcggagtgcggcgagtgtgagttctgtcgttctggcaaaactaacctctgtgttgcggttcgcgaaacccagggtaaaggcttgatgccagacggcaccacccgtttttcttacaacgggcagccgctttatcactacatgggatgctcaacattcagtgaatacaccgtggtcgcggaagtgtctctggccaaaattaatccagaagcaaaccatgaacacgtctgcctgctgggctgtggcgtgaccaccggtattggcgcggtgcacaacacagctaaagtccagccaggtgattctgttgccgtgtttggtcttggcgcgattggtctggcagtggttcagggcgcgcgtcaggcgaaagcgggacggattatcgctatcgataccaacccgaagaaattcgatctggctcgtcgcttcggtgctaccgactgcattaacccgaatgactacgacaaaccgattaaagatgtcctgctggatatcaacaaatggggtatcgaccatacctttgaatgcatcggtaacgtcaacgtgatgcgtgcggcgctggaaagtgcgcaccgcggctggggtcagtcggtgatcatcggggtagcaggtgccggtcaggaaatctccacccgaccattccagttggtcaccggtcgcgtatggaaaggttccgcgtttggcggcgtgaaaggtcgttcccagttaccgggtatggttgaagatgcgatgaaaggtgatatcgatctggaaccgtttgtcacgcataccatgagccttgatgaaattaatgacgccttcgacctgatgcatgaaggcaaatccattcgaaccgtaattcgttactgatttcccgcaggtataccccgtccacttcagacggggttcttaatactctccctgggcagccgtccgggggattaaccgtgagataatgactgatggaactcattgaaaaacatgccagctttggcggctggcaaaatgtgtatcggcattattcccaatcactgaaatgtgaaatgaatgtcggcgtctatctcccaccaaaagccgcgaatgaaaaattgccggtgttgtactggctttcaggcctgacttgcaacgagcagaatttcattactaaatcggggatgcagcgttacgcggctgagcacaacattattgttgttgcgccggacaccagtccgcgaggcagtcatgtcgcagatgctgaccgttacgatctcgggcaaggtgccgggttttacctgaacgcgacgcaagcgccgtggaatgaacattacaaaatgtatgactatatccgcaacgagctgccggatttagtgatgcatcattttccggcaacggccaaaaagtctatctctggtcattctatgggcgggctgggcgcgctggtgctggcgttacgtaacccagatgaatatgtcagcgtctcggcgttttcgcccattgtctccccatcgcaagtgccgtggggacagcaagcctttgctgcatatcttgctgaaaataaagatgcctggttggattacgacccggtgagtcttatttcacaaggtcaacgcgttgcggaaatcatggttgatcaggggttgagtgatgatttttacgcagaacagctgcggactccaaatcttgaaaagatctgccaggagatgaatatcaagacgttaatccgttatcacgagggttatgatcacagctattattttgtctccagttttattggcgagcatattgcctaccacgccaataaactgaatatgcgttgataatagtgcacgactgccggatgcggcgtgaacgccttatccggcctacacttcgcccgtaaaccgtaggcctgataagacgcgcaaagcgtcgcatcaggcatggcgcacgactgccggatgcggcgtgaacgccttatccggtctacacttcgcccataaaccgtaggcctgataagacgcgcatagcgtcgcatcaggcatctgcgcacgacttatctatgtttaccacatcaaatcatcaggcactttgaagtcggcatacgggtcgtcttcatcctgctcttcggcgctgagcgcgctgtgcaacactatactgctggcatcgcgctgagcaattttatcggcaacgctggcgggaataatggcgtattcaccttcgctattgttatcgaccaacaagcgggcaatcgccagacgaccattaatcaactgcgcctgtgtgagcttatcgacaaaaatcttcttaatcagattgccgtcagtgaagttaaaaccaatatcgccattggcaatggtgattcggttcatttcaataagctgcttcacctgagctttatattctttcgccaacgccgcttgtttttgctgttcgctaagctgtttatcacgctcaagctgtgcctttttattttcttctaccgccgcccgagcttcacgcgcctgaacgcgcgattttttcgccgttctctccaccttcgccgcttttttgctggtgactaatcctgctttgagcaactgctcttgtaaggtaagttttgccatctttgtttccaaacccgctgaataatttttagcgagtatacctgtaaacgccgtaggtgtattggggtgaagtccctttcgcccctgcctaataaggcacgtcaggcatcggcgcacggctgtattcgtgatctccggctcatcaaaataaacaccgccaacccagcaacaagaatacccggtgcagaagcggccattacgccgaccgtgccagtgcctaatgccagcattttcccggccagtaacggaccgctcatagcccccagacgccctaccgccacggctgttcccacacctgttgcgcggatctgcgaactgtaaaacaacggtgccagggcatacaaaacgctttgcccacctgtcgcaaacaaccccgcgacaaatcccgccagcaacataccgttaaatgacgacaccgttccaagcgccagcagcgaagctaacatgccgctataaatcagtagcgacatggttactggacgcagcttatccatcaatgcgcccaacattaacgtcccgcttgccgcccccatttgcagggcaaacatcacccctgccgcctgcgatggctggaatccttgctccaccaaaagtagcggtagccagttgatcaacatgtagaccaccagcagagtgaagaaataacacaaccacagcagcagcgtcgcggttgccgtttctggcgcaaataaggcacgcagtggtggcgcagactgtttttcgccagcgaaaaccgccgactccggcagccagcgcattaatagcggcaccagaatcaacggcaccacaccacctacccaaaacaccgtttgccatgctaagtttgcccccgcgaaacccagtgtcgccgccagcgccgcgccaatgggaacaccgcaatacatcaggctcactgccgtcccacgaaaacgtggacccgcggcttcagacgtcagggcgataagattcggcaacgccgcccccagcccgacaccggtcatcagccgcgcaaagaccagtgaggggaaatcccaggcaatcgccgttgccagtgagaacaaaccaaacagcgcaactgagccaatcaaaatacgcttacgaccataacggtccgccagcattccgccaaccaacgcgccgggtagcaaaccgagtattccggcgctaaatatccagcccatttgcattttatcgagtgcgaaagcctgggcgatgccacccgccgcaatgccagccgcctgaagatccagcccttccatcagagcgaccaaaaaacaaagcccgatggtcagcatcaggcgggatgaagatgatgaaggggtacgagtcgacataagatgaacctcattgcgagtgcaggtgaatcgcgactgatacattgccagccgcgtaatcagaactattgaatgactgcctacgggcgaaatgtaggccggataaggcgttcacgccgcatccggcagtcgtgtgcagatgcctgatgcgacgcttgcgcgtcttatcaggcctacaaattcccgcgccatccgtaggccggataaggcgtttacgccgcatccggcagtcgtgcgcagatgcctgatgcgacgctaacgcgtcttatcaggcctacaaattctcgcgccatccgtaggccggacaagacgttcacgccgcatccggcagtcgtgctgggatgggcatacaaaatgcacattctcggcagtcaatattatttgttgttgcgcagatccagcgccacgtcgacgatcatatcctcctggccgccaaccatccggcgtttgcccagctcaacgagaatatccaccgcgcttaagccataacgcgccgccgccgtttcacagtgacgcaggaagctggagtaaacaccagcgtatcccagcgccagcgtttcgcgatcgactcgtaccggtcgatcctgcaacggacgcaccaggtcgtcagcggcatccattaacgcatagagatcggtcccatgctgccagcccagtttatccgcggcggcaataaatacttccagcggcgcgttacctgcgcccgcgcccattcccgcgaggctggcgtcgattcggtcgcagccctcttccaccgcctcgatagagttcgccacgccaagactcaggttatggtgagcgtgcatgccggtttgcgtttctggtttcaacactgctttcagggcgcggaaacggtcacggatatcgctcatgttcatcgcaccgccagaatccaccacataaatacaggtcgcaccgtagccttccatcagctttgcctgctttgcgagattctctggcgtggtcatatggctcatcatcagaaaaccaacggtgtccattccgagctcgcgggcatactgaatatgctgggcggaaacatcagcttcggtacagtgcgtagccacacgaaccacccgcgcgccagcttgccaggcatttttcagatcgtgaatagtgccgattcctggcagcaacaacgtcgcgattttggcgtgcttcaccacatccgccgctgcttcaatccattcaaggtcgctatgtgcgccgaaaccatagttaaagctggaaccctgcaaaccgtcgccgtgggccacttcaatcgaatccacgtgggcatcgtccagtgctttggcaatctggcgaacgttttccagcgaatactgatgacgaatggcgtgcataccgtcacgcaacgtgacgtccgagatataaagttttttaccgttcatgccgcttctcctgccttgcgcgccagtgactgggccattttttccgctgtcgccagcgcactggaagtcataatgtcgaggttgcccgcataggcaggcagataatgcgctgcgccttcgacttccagccagaccgctgttttcagtccggagaattgccccacgcccggtaaattgaccggtttatcctgcgggataacttcaaactgcacgcgctgtttcaggcgataacccggtacgtaagcctgcaccgcctcagccatttcattgattgaggcttcgatatcatcttgtgaagcttcgtcgctcaatacatacaccgtgtcacgcatcatcaacggtggctctgctgggttaagcacaataatcgccttcccttttgccgcgccgcccaccacttcaatggctcgggaagtggtttccgtaaattcatcgatattggcacgcgtgccaggtccggcagatttactggcgatagaagcgataatttcggcgtaatgaacacgcgccacgcgtgaaactgccgccaccattggaatggtggcctggccgccgcaggtgaccatgttgacgttcagttgatcgacgttcgcctcgaggttaaccaccggcacgcagtaagggccgatggcagcaggcgtcaggtcaattaagcgaatatccggtttcgcttcgcgtaaagcggcatcgtttttcacatgagcaccggcgctggtcgcatcaaatacaatgtcgatatcagcaaattcaggcatgttcatcagtccgatcaccccttcatgggtggtggcgacgcccatacgtctggcgcgcgccagaccgtcggactgaggatcaatgccaaccatcaccgccatctccagatgctgaccgtgacgcaaaattttaatcatcagatcggtgccaatgttgccagaaccgataatggcgactttacgcttactcatgacagacttccttttggggctgcgcttgaaaatgtcgcagcaactgaacctatgccttcaatatgggcttcaaaacgatcgcccgcattcaccgccaccatcggacctaatgccccggtaagaatgatatctccggtgcgcagcggttcacccagactggccattttgcgtgccagccagacggccgcattaagcggatgtcccaggcattcgctgccgcgcccgctagaaacctcttcgttattacgcgtcatcttcatggcgcagtttttcaggtctaaccccgccggacgttgcgccggaccgccgatgacatacaccccacaggaggcgttatctgccacggtatcgacaaactgaatcgaccagtcgcgaatgcggctccccaccacttccagcgccggaagtacccattcaatggcgttatacaattcgtcgaaggtgatatcggttgcgggcaaatcgcggttcaacaccagtgcgatctccgcttcaatgcggggttgcagaacacgggaaaaaggaatgatttcgttatcgccataacacatgtcggcaaataatgtcccgaaatccggttgatcaacgcccagttgttgctgcactttcggatgcgtcaggcccactttacgccctaccacgcgacgcccctgcgcaacgtcatattgcacatttatgtgctgaatggcgtaggccgcttcagcgttatcgataccaatcagatcgcgcagcggcgcaatcgcttcgccctgctctgcggcgcggcgtaaatccgccgccagttgctcaagagtatgcttcgtcatgaccattccttaagggcgtgcgaggaaattcagcaccaattgattgaaagcgtcggcatgttcccactgcgcccagtgaccacagtcgcggaagatatgcagttcagaaccggcaatgccggacagcagacgcagacccgcatccatcggcacaaagcggtcgttgcgcccccagacaatcagggtttgcgctttgatttccgccagacgtgggccaaaatccgggaactgtttcggattagcttccaggctcttaacgaagttttccaggtgatcgcggcgcgacagcatattattcaggcgcgcttcaaacagggcgtcggtcaaatcgctggtatcgaaaacgaagatatccatcatcagcttcaggttttcgatagtcggctgacgataaagctgattcagtcgcttaataccttcggttggcatcggcgtaaacaaactcatgccgcccgtaccgccgcccatcagcaccagtttgccgacgcgctccggccagttaagggtgaacgccacagaactatggccgcccatcgagttgcccagcaggtggattttggcgatatccagttgatccaccacgcttttcaggattcgtgcattaagatccgatcgtgaaccactattaacgatcgaatcgcttttgccccaacccggacaatccagcaggatcacccgatagcccgcctctaccagcggatcgatattgcggctgaagttcgcccagccagtagcacccgggccggaaccatgcagcaggacaacggtttcgtcgccttgtccgcagtcattaaaatggatgcgcagcgttttacccgcttcttctacattcagaaaacggctggtggcggcttcggtttgtggctgataactcatcatcttctcctgcatattcagttctctgttctggcgcttaacgagccaaatccggcaatccactccgggattgggcggtaataacgcccttcgctacgccagttgccaaatgcagaaatagcggcaaaagcggcgacccaggttttgatttcatgtgtcgactttccggcaatggcggaaagctcttcgttactgacggcatccagttcctgtatgcgtccctgctccagcaaagtcatgaactggttatcccaaatcgggttgagcggatgcagagttctctgatcctcaacaaacttctcagcggcgctaatcacccgttgctgacgcaattcgcgctcactggcgggtaaatctttcccgctccccaacagacggtcgcgcatatgggcatcggctttcgccagttcgggcaccggcggctgatgggaaagcccaccggaacccaggaacagcacgcgtttattgagagtgctggtgaaacgtccaatggcttcacccaacatgcgggtacgctggaaaccgggcagcggcgtggcgacaccgttgatgaacacaggcagaactggcaccttatccagcccgccgagcaggaactccagtggctgggcgaacccgtggtccacctgcatacagtaagaaacggcaagatcgatcccgctcttcatgacggcatgcgcacaggcctccgccagctccacaggcacgggcagctctcctgccgcactgccgaaatcaccaattgccgtcgccccaacgcctaaacagaacggtggcatcacgtcatagaaaaagccgttgtagtgatctggcgcaaacagcaccaccagttcaggggagaaagccgcaatacgctcgcgggcgctggcaatcacgccattgacctcatcgagtacctcttgcgccgggtcgacatatcccaccagcggcgagtgggaaagacagtgaagataagcgtgcatatcaggctaccttttcgacagaaacgtcggcatcagggcgggtcagcgtcatcaccgacgccagtttattcagcgtattacccagggtttgcggaatggcggtggcggcaacaaagcgatccgggcgcatcaccaccagcgaagcattatgttgtgcgaaccagctacgcaggcgaccttgcgtatcccccacgcgtagtacgccgtcgtggttatcctgtgcggtatgaatttgcacttccggcaccacctgaatgaagcgggtgcccaacgcgcgccactgctggatttgctcgtcgctcatcccccacagtggattgcatccccagccaattaccgcgaagttcgcgccgatcgcgttatcgagcagcgtcacgtcgccgttttccagcgtgactttcggctgaataaacatcttgccgaccggagagtgcttcgcttcgccctctcgcaccagcgcaccgccgtaatattgcggcatcggcttgaagcgcatttcgaggaagtagcgttttactggcggcagataattcaacagccaggaaacgccgtcacgtaacgtaccctgccagcgtttcggcggagccagcacgttgcccgccgtcacggacaggtcaatcatcgctttggcgtgatcgcgtcgttcttgttgataggtatcgagcagcgcatcacgggctttcccctggataaccaacgccagtttccaggcgaggttaaaggcgtcgcgcataccgctgttatagccctgcccctgccataccggcatgatgtgcgcggcatcgcccgccagcagtacgcgatcaatacggaaacgttgcgccagtcgcgcgttgtgggtgtagacacgctggcgaatcaattcaacattgtccggattaggcagcactttgcttaacagcttgcgcatattttgcggctcacgcagctgttcttcggtttctcctggcatcaccataaattcaaagcgacgtaccgcatgaggtagcgcggcagaaacatacgggcgcaccggatcgcaacacaaatagatatgcggcgtacttaacggatcgttggcgatatctaccacaatccactgatttggcgcagttttaccttcaaacggcacattcagggtgcgacggacaaaacttgccccaccatcacaggccaccagccactgggctttgactatttcccgctgcccttctgccgttttcaggtgcaaggtcacttcgtcatcttgctgactgaaggcctccagctcgcgggaaaacaagcagcgcacattcggaaaacgcgacaccccttccagcatcaccgcatcgacctgcggctgaataaaggcgttacggcgcggccagccaaattcatcggtcattggctgaatatcagcaaaacagcggcctttcggggtgagaaaacgcatcgcgtgccacggcgtagtgtgcggcagaacatcatcgaccaggccgaccgactgcatggtgcgcagcgcctcgtcatcaataccaatcgcacgcgggtagtcgatcaacttatcgagtttctccaccaccagcacgtcaatgcccatctggccgagatagttcgccatcatcagcccaaccgggccggcaccagcgatcgccacctgaacgctatggttaacagcaggctggatgtcagggtgttgtattgccatttcagtacctcacgactcggacaaaatgtcgttgcgcgcacagtacagcgcaacttattttgttaaaaacatgtaaatgattttttattgtgcgctcagtataggaagggtgttttcggctacaatcaaaacatgcccgaatgtgcaccaggtgcaccacgttgttttaactatagaaatgtcaattaatatgcagaacaatgagcagacggaatacaaaaccgtgcgcggcttaacccgcggtctaatgttattaaatatgttaaataaacttgatggcggtgccagcgtcgggctgctggcggaactcagcggcctgcatcgcaccactgtgcggcgactgctggagacgctgcaggaagagggatatgtccgccgtagcccctccgatgatagttttcgactgaccatcaaagtgcggcaattaagcgaaggatttcgtgacgaacagtggatttctgcactggcggccccactgctgggcgatctgttgcgcgaagtggtatggccgacagatgtgtccacgctggatgttgatgcaatggtggtacgcgaaaccactcaccgtttcagccgcttatcctttcaccgggcaatggtcgggcgacgtttgccgcttctgaaaaccgcctcgggcctgacctggctggccttttgcccggaacaagaccgcaaggaattaatcgaaatgttagcctcccgccccggtgatgactatcaactggcacgggaaccgttaaagctggaagccattctggcgcgcgcgcgcaaagagggttacggacagaactaccgcggctgggatcaggaggagaagatcgcctctatcgccgtaccgctgcgcagtgaacaacgggtgattggctgtctgaatctggtgtatatggcgagcgcaatgaccattgaacaggcagcggaaaagcatcttccggcgctacaacgggtagcaaaacagatcgaagaaggggttgaatcgcaggctattctggtggccggaaggcgaagcggcatgcatttacgttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaactaggcaccccaggctttacactttatgcttccggctggtataatgtgtggacctgtagaaataattttgtttaactttaataaggagatataccatgggaggcagtactgttgcaccaactacaccgttggcaaccggcggtgcgctccgcaaagtgcgacaagccgtctttcccatctacggaaaccaagaagtcaccaaatttctgctcatcggatccattaaattctttataatcttggcactcacgctcacgcgtgataccaaggacacgttgattgtcacgcaatgtggtgccgaagcgattgcctttctcaaaatatacggggtgctacccgcagcgaccgcatttatcgcgctctattccaaaatgtccaacgccatgggcaaaaaaatgctattttattccacttgcattcctttctttacctttttcgggctgtttgatgttttcatttacccgaacgcggagcgactgcaccctagtttggaagccgtgcaggcaattctcccgggcggtgccgcatctggcggcatggcggttctggccaagattgcgacacactggacatcggccttattttacgtcatggcggaaatatattcttccgtatcggtggggctattgttttggcagtttgcgaacgacgtcgtcaacgtggatcaggccaagcgcttttatccattatttgctcaaatgagtggcctcgctccagttttagcgggccagtatgtggtacggtttgccagcaaagcggtcaactttgaggcatccatgcatcgactcacggcggccgtaacatttgctggtattatgatttgcatcttttaccaactcagttcgtcatatgtggagcgaacggaatcagcaaagccagcggcagataacgagcagtctatcaaaccgaaaaagaagaaacccaaaatgtccatggttgaatcggggaaatttctcgcgtcaagtcagtacctgcgtctaattgccatgctggtgctgggatacggcctcagtattaactttaccgaaatcatgtggaaaagcttggtgaagaaacaatatccagacccgctagattatcaacgatttatgggtaacttctcgtcagcggttggtttgagcacatgcattgttattttcttcggtgtgcacgtgatccgtttgttggggtggaaagtcggagcgttggctacacctgggatcatggccattctagcgttacccttttttgcttgcattttgttgggtttggatagtccagcacgattggagatcgccgtaatctttggaacaattcagagtttgctgagcaaaacctccaagtatgcccttttcgaccctaccacacaaatggcttatattcctctggacgacgaatcaaaggtcaaaggaaaagcggcaattgatgttttgggatcgcggattggcaagagtggaggctcactgatccagcagggcttggtctttgtttttggaaatatcattaatgccgcacctgtagtaggggttgtctactacagtgtccttgttgcgtggatgagcgcagctggccgactaagtgggctttttcaagcacaaacagaaatggataaggccgacaaaatggaggcaaagaccaacaaagaaaagtagttaacctaggctgctgccaccgctgagcaataagactcctgttgatagatccagtaatgacctcagaactccatctggatttgttcagaacgctcggttgccgccgggcgttttttattggtgagaat

SEQ ID NO: 5 - DNA, 17898 bp, Escherichia coli
  source 1..17898: mol_type = genomic DNA
ttattgcacgaagcgcgaggtaacgtactggctgtaatcattcgctacagccgggaccttgccctgctctttcaaaaactgcgcggtgtcgatgatcgctttgttcaccggtccggtcagttctgccgtttgttgctgcggcgtcagataggtattccccttcaccagccccggaacgtcaccttcaggcacgccgcttaaccgcgccagtttgctgatgttttccggctgtttcagccacgcgtctgggttagcaatgtacggttgctgagcatcgatggcgcttttggcgaacgctttcaccacctcaggatgtttctcggcaaaatctttgcgcaccacccagacgtccagcgttggcgcaccccactgcccgacctgttcagaatcggtcagcactttgccgtctttttccagggcgttaaccgccggtgcccagacataagcaccatcaatatctccccgctgccatgcagcgataatcgcgggcggctgcaggttcacaatctccacttgcccaggtttaatgccccagtgtttcagcgctgccagcaggctgtagtgggtggtagagataaacggtacggcgatgcgtttgccaatcagatcttccggtttgctgatagttttctttaccaccagcgcttctgagttacccagttttgacgccagcaagaagacttcaatcggcacctgttggctggctgcaaccgctaacgggctggaaccaaggttgccgatttgcacgtcgccagaagccagcgcccgcacgatgctggctccgctgtcaaacttacgccagtccacggttgctccgctttctttagcaaaggtgttgtcggcctgagccactttcgccggttcggctgaggtttgatacgccacggtgacgttcaccgcctgtgcctgaaaagcgatgaatgccagtgcggcaagaagtgtgtttcgcgatgaaattgccatgattgtctgctcccctgtcttgttatgggagcagtattcaggaataaaaacattcattaaaagaattagtcgttatcgcacagatgattttattcttagcaaaaaaacggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaactgcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacaggctccggaagacggttgttgcgcacgtattcggtgaacgcactatggcgacgctggggcgtcttatgagcctgatgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacctagaacgtaatttacctgccggaacttattcactccgacaagaacttatccgtacaggagattaaaatgataaaacggacgttattagcggcggccatttttagcgcattgcccgcttatgccgggttaacttccattaccgcgggctacgattttaccgattattctggcgatcatggcaaccgtaatttagcgtatgctgaactggtggcgaaagttgaaaacgcaacgctgctttttaatctttcccaggggcgtcgtgattatgaaactgaacatttcaatgccactcgcggtcagggtgcggtctggtataaatggaataactggctgacaacccgaacgggtattgcctttgcggataatacgccggtctttgcccgccaggattttcgtcaggatattaacctggccctgttgccaaaaacgcttttcacgaccggttatcgctacactaaatattacgatgatgtcgaagtcgatgcctggcaaggcggcgtatcactctatactggcccggtgatcaccagctaccgctatacccattatgactccagcgatgcaggtggtagttatagcaatatgatttccgtgcgtctgaatgacccgcgcggcactggttatacgcaactatggctaagccgcggaacaggcgcttacacctatgactggacgccagaaacacgctacggcagcatgaagagcgtcagtctgcaacgtattcaaccgctgactgagcaacttaatctcgggctgacggcaggtaaagtgtggtacgacaccccaaccgatgattataacggtctgcaacttgcagcccatctgacctggaaattctgattccttctgccgcccgctatccggggcggccttccctgccgattagcccccccctttcctctttgttttccgaccacattcaccggataaattttattctccagtgttatatactataggggggtatgcattgacatatagcatacccccctatagtatattgcgtgcagataatgaggtgcgaaatgcccagtactccggaagagaagaaaaaggtccttactcgagttcgtcgtattcgagggcagattgatgctctggaacggtcgctggagggtgatgccgaatgccgtgccatactccaacagatcgctgccgttcggggcgcggctaatgggctgatggcagaagtgcttgaaagccatatccgggaaacgtttgaccgaaatgactgctacagccgcgaagtcagccaatccgttgacgacactattgaactggttcgagcctatcttaaatagctgaatctattaccatattgaggaagagcgagagatgaaatcacgtgctgccgttgcatttgctcccggtaaaccgctggaaatcgttgaaattgacgttgcaccaccgaaaaaaggtgaagtgctgattaaagtcacccataccggcgtttgccataccgacgcatttaccctctccggggatgacccggaaggtgtattcccggtggttctcggtcacgaaggggccggcgttgtggttgaagtcggtgaaggcgtaaccagcgtcaaacctggcgaccatgtgatcccgctttacaccgcggagtgcggcgagtgtgagttctgtcgttctggcaaaactaacctctgtgttgcggttcgcgaaacccagggtaaaggcttgatgccagacggcaccacccgtttttcttacaacgggcagccgctttatcactacatgggatgctcaacattcagtgaatacaccgtggtcgcggaagtgtctctggccaaaattaatccagaagcaaaccatgaacacgtctgcctgctgggctgtggcgtgaccaccggtattggcgcggtgcacaacacagctaaagtccagccaggtgattctgttgccgtgtttggtcttggcgcgattggtctggcagtggttcagggcgcgcgtcaggcgaaagcgggacggattatcgctatcgataccaacccgaagaaattcgatctggctcgtcgcttcggtgctaccgactgcattaacccgaatgactacgacaaaccgattaaagatgtcctgctggatatcaacaaatggggtatcgaccatacctttgaatgcatcggtaacgtcaacgtgatgcgtgcggcgctggaaagtgcgcaccgcggctggggtcagtcggtgatcatcggggtagcaggtgccggtcaggaaatctccacccgaccattccagttggtcaccggtcgcgtatggaaaggttccgcgtttggcggcgtgaaaggtcgttcccagttaccgggtatggttgaagatgcgatgaaaggtgatatcgatctggaaccgtttgtcacgcataccatgagccttgatgaaattaatgacgccttcgacctgatgcatgaaggcaaatccattcgaaccgtaattcgttactgatttcccgcaggtataccccgtccacttcagacggggttcttaatactctccctgggcagccgtccgggggattaaccgtgagataatgactgatggaactcattgaaaaacatgccagctttggcggctggcaaaatgtgtatcggcattattcccaatcactgaaatgtgaaatgaatgtcggcgtctatctcccaccaaaagccgcgaatgaaaaattgccggtgttgtactggctttcaggcctgacttgcaacgagcagaatttcattactaaatcggggatgcagcgttacgcggctgagcacaacattattgttgttgcgccggacaccagtccgcgaggcagtcatgtcgcagatgctgaccgttacgatctcgggcaaggtgccgggttttacctgaacgcgacgcaagcgccgtggaatgaacattacaaaatgtatgactatatccgcaacgagctgccggatttagtgatgcatcattttccggcaacggccaaaaagtctatctctggtcattctatgggcgggctgggcgcgctggtgctggcgttacgtaacccagatgaatatgtcagcgtctcggcgttttcgcccattgtctccccatcgcaagtgccgtggggacagcaagcctttgctgcatatcttgctgaaaataaagatgcctggttggattacgacccggtgagtcttatttcacaaggtcaacgcgttgcggaaatcatggttgatcaggggttgagtgatgatttttacgcagaacagctgcggactccaaatcttgaaaagatctgccaggagatgaatatcaagacgttaatccgttatcacgagggttatgatcacagctattattttgtctccagttttattggcgagcatattgcctaccacgccaataaactgaatatgcgttgataatagtgcacgactgccggatgcggcgtgaacgccttatccggcctacacttcgcccgtaaaccgtaggcctgataagacgcgcaaagcgtcgcatcaggcatggcgcacgactgccggatgcggcgtgaacgccttatccggtctacacttcgcccataaaccgtaggcctgataagacgcgcatagcgtcgcatcaggcatctgcgcacgacttatctatgtttaccacatcaaatcatcaggcactttgaagtcggcatacgggtcgtcttcatcctgctcttcggcgctgagcgcgctgtgcaacactatactgctggcatcgcgctgagcaattttatcggcaacgctggcgggaataatggcgtattcaccttcgctattgttatcgaccaacaagcgggcaatcgccagacgaccattaatcaactgcgcctgtgtgagcttatcgacaaaaatcttcttaatcagattgccgtcagtgaagttaaaaccaatatcgccattggcaatggtgattcggttcatttcaataagctgcttcacctgagctttatattctttcgccaacgccgcttgtttttgctgttcgctaagctgtttatcacgctcaagctgtgcctttttattttcttctaccgccgcccgagcttcacgcgcctgaacgcgcgattttttcgccgttctctccaccttcgccgcttttttgctggtgactaatcctgctttgagcaactgctcttgtaaggtaagttttgccatctttgtttccaaacccgctgaataatttttagcgagtatacctgtaaacgccgtaggtgtattggggtgaagtccctttcgcccctgcctaataaggcacgtcaggcatcggcgcacggctgtattcgtgatctccggctcatcaaaataaacaccgccaacccagcaacaagaatacccggtgcagaagcggccattacgccgaccgtgccagtgcctaatgccagcattttcccggccagtaacggaccgctcatagcccccagacgccctaccgccacggctgttcccacacctgttgcgcggatctgcgaactgtaaaacaacggtgccagggcatacaaaacgctttgcccacctgtcgcaaacaaccccgcgacaaatcccgccagcaacataccgttaaatgacgacaccgttccaagcgccagcagcgaagctaacatgccgctataaatcagtagcgacatggttactggacgcagcttatccatcaatgcgcccaacattaacgtcccgcttgccgcccccatttgcagggcaaacatcacccctgccgcctgcgatggctggaatccttgctccaccaaaagtagcggtagccagttgatcaacatgtagaccaccagcagagtgaagaaataacacaaccacagcagcagcgtcgcggttgccgtttctggcgcaaataaggcacgcagtggtggcgcagactgtttttcgccagcgaaaaccgccgactccggcagccagcgcattaatagcggcaccagaatcaacggcaccacaccacctacccaaaacaccgtttgccatgctaagtttgcccccgcgaaacccagtgtcgccgccagcgccgcgccaatgggaacaccgcaatacatcaggctcactgccgtcccacgaaaacgtggacccgcggcttcagacgtcagggcgataagattcggcaacgccgcccccagcccgacaccggtcatcagccgcgcaaagaccagtgaggggaaatcccaggcaatcgccgttgccagtgagaacaaaccaaacagcgcaactgagccaatcaaaatacgcttacgaccataacggtccgccagcattccgccaaccaacgcgccgggtagcaaaccgagtattccggcgctaaatatccagcccatttgcattttatcgagtgcgaaagcctgggcgatgccacccgccgcaatgccagccgcctgaagatccagcccttccatcagagcgaccaaaaaacaaagcccgatggtcagcatcaggcgggatgaagatgatgaaggggtacgagtcgacataagatgaacctcattgcgagtgcaggtgaatcgcgactgatacattgccagccgcgtaatcagaactattgaatgactgcctacgggcgaaatgtaggccggataaggcgttcacgccgcatccggcagtcgtgtgcagatgcctgatgcgacgcttgcgcgtcttatcaggcctacaaattcccgcgccatccgtaggccggataaggcgtttacgccgcatccggcagtcgtgcgcagatgcctgatgcgacgctaacgcgtcttatcaggcctacaaattctcgcgccatccgtaggccggacaagacgttcacgccgcatccggcagtcgtgctgggatgggcatacaaaatgcacattctcggcagtcaatattatttgttgttgcgcagatccagcgccacgtcgacgatcatatcctcctggccgccaaccatccggcgtttgcccagctcaacgagaatatccaccgcgcttaagccataacgcgccgccgccgtttcacagtgacgcaggaagctggagtaaacaccagcgtatcccagcgccagcgtttcgcgatcgactcgtaccggtcgatcctgcaacggacgcaccaggtcgtcagcggcatccattaacgcatagagatcggtcccatgctgccagcccagtttatccgcggcggcaataaatacttccagcggcgcgttacctgcgcccgcgcccattcccgcgaggctggcgtcgattcggtcgcagccctcttccaccgcctcgatagagttcgccacgccaagactcaggttatggtgagcgtgcatgccggtttgcgtttctggtttcaacactgctttcagggcgcggaaacggtcacggatatcgctcatgttcatcgcaccgccagaatccaccacataaatacaggtcgcaccgtagccttccatcagctttgcctgctttgcgagattctctggcgtggtcatatggctcatcatcagaaaaccaacggtgtccattccgagctcgcgggcatactgaatatgctgggcggaaacatcagcttcggtacagtgcgtagccacacgaaccacccgcgcgccagcttgccaggcatttttcagatcgtgaatagtgccgattcctggcagcaacaacgtcgcgattttggcgtgcttcaccacatccgccgctgcttcaatccattcaaggtcgctatgtgcgccgaaaccatagttaaagctggaaccctgcaaaccgtcgccgtgggccacttcaatcgaatccacgtgggcatcgtccagtgctttggcaatctggcgaacgttttccagcgaatactgatgacgaatggcgtgcataccgtcacgcaacgtgacgtccgagatataaagttttttaccgttcatgccgcttctcctgccttgcgcgccagtgactgggccattttttccgctgtcgccagcgcactggaagtcataatgtcgaggttgcccgcataggcaggcagataatgcgctgcgccttcgacttccagccagaccgctgttttcagtccggagaattgccccacgcccggtaaattgaccggtttatcctgcgggataacttcaaactgcacgcgctgtttcaggcgataacccggtacgtaagcctgcaccgcctcagccatttcattgattgaggcttcgatatcatcttgtgaagcttcgtcgctcaatacatacaccgtgtcacgcatcatcaacggtggctctgctgggttaagcacaataatcgccttcccttttgccgcgccgcccaccacttcaatggctcgggaagtggtttccgtaaattcatcgatattggcacgcgtgccaggtccggcagatttactggcgatagaagcgataatttcggcgtaatgaacacgcgccacgcgtgaaactgccgccaccattggaatggtggcctggccgccgcaggtgaccatgttgacgttcagttgatcgacgttcgcctcgaggttaaccaccggcacgcagtaagggccgatggcagcaggcgtcaggtcaattaagcgaatatccggtttcgcttcgcgtaaagcggcatcgtttttcacatgagcaccggcgctggtcgcatcaaatacaatgtcgatatcagcaaattcaggcatgttcatcagtccgatcaccccttcatgggtggtggcgacgcccatacgtctggcgcgcgccagaccgtcggactgaggatcaatgccaaccatcaccgccatctccagatgctgaccgtgacgcaaaattttaatcatcagatcggtgccaatgttgccagaaccgataatggcgactttacgcttactcatgacagacttccttttggggctgcgcttgaaaatgtcgcagcaactgaacctatgccttcaatatgggcttcaaaacgatcgcccgcattcaccgccaccatcggacctaatgccccggtaagaatgatatctccggtgcgcagcggttcacccagactggccattttgcgtgccagccagacggccgcattaagcggatgtcccaggcattcgctgccgcgcccgctagaaacctcttcgttattacgcgtcatcttcatggcgcagtttttcaggtctaaccccgccggacgttgcgccggaccgccgatgacatacaccccacaggaggcgttatctgccacggtatcgacaaactgaatcgaccagtcgcgaatgcggctccccaccacttccagcgccggaagtacccattcaatggcgttatacaattcgtcgaaggtgatatcggttgcgggcaaatcgcggttcaacaccagtgcgatctccgcttcaatgcggggttgcagaacacgggaaaaaggaatgatttcgttatcgccataacacatgtcggcaaataatgtcccgaaatccggttgatcaacgcccagttgttgctgcactttcggatgcgtcaggcccactttacgccctaccacgcgacgcccctgcgcaacgtcatattgcacatttatgtgctgaatggcgtaggccgcttcagcgttatcgataccaatcagatcgcgcagcggcgcaatcgcttcgccctgctctgcggcgcggcgtaaatccgccgccagttgctcaagagtatgcttcgtcatgaccattccttaagggcgtgcgaggaaattcagcaccaattgattgaaagcgtcggcatgttcccactgcgcccagtgaccacagtcgcggaagatatgcagttcagaaccggcaatgccggacagcagacgcagacccgcatccatcggcacaaagcggtcgttgcgcccccagacaatcagggtttgcgctttgatttccgccagacgtgggccaaaatccgggaactgtttcggattagcttccaggctcttaacgaagttttccaggtgatcgcggcgcgacagcatattattcaggcgcgcttcaaacagggcgtcggtcaaatcgctggtatcgaaaacgaagatatccatcatcagcttcaggttttcgatagtcggctgacgataaagctgattcagtcgcttaataccttcggttggcatcggcgtaaacaaactcatgccgcccgtaccgccgcccatcagcaccagtttgccgacgcgctccggccagttaagggtgaacgccacagaactatggccgcccatcgagttgcccagcaggtggattttggcgatatccagttgatccaccacgcttttcaggattcgtgcattaagatccgatcgtgaaccactattaacgatcgaatcgcttttgccccaacccggacaatccagcaggatcacccgatagcccgcctctaccagcggatcgatattgcggctgaagttcgcccagccagtagcacccgggccggaaccatgcagcaggacaacggtttcgtcgccttgtccgcagtcattaaaatggatgcgcagcgttttacccgcttcttctacattcagaaaacggctggtggcggcttcggtttgtggctgataactcatcatcttctcctgcatattcagttctctgttctggcgcttaacgagccaaatccggcaatccactccgggattgggcggtaataacgcccttcgctacgccagttgccaaatgcagaaatagcggcaaaagcggcgacccaggttttgatttcatgtgtcgactttccggcaatggcggaaagctcttcgttactgacggcatccagttcctgtatgcgtccctgctccagcaaagtcatgaactggttatcccaaatcgggttgagcggatgcagagttctctgatcctcaacaaacttctcagcggcgctaatcacccgttgctgacgcaattcgcgctcactggcgggtaaatctttcccgctccccaacagacggtcgcgcatatgggcatcggctttcgccagttcgggcaccggcggctgatgggaaagcccaccggaacccaggaacagcacgcgtttattgagagtgctggtgaaacgtccaatggcttcacccaacatgcgggtacgctggaaaccgggcagcggcgtggcgacaccgttgatgaacacaggcagaactggcaccttatccagcccgccgagcaggaactccagtggctgggcgaacccgtggtccacctgcatacagtaagaaacggcaagatcgatcccgctcttcatgacggcatgcgcacaggcctccgccagctccacaggcacgggcagctctcctgccgcactgccgaaatcaccaattgccgtcgccccaacgcctaaacagaacggtggcatcacgtcatagaaaaagccgttgtagtgatctggcgcaaacagcaccaccagttcaggggagaaagccgcaatacgctcgcgggcgctggcaatcacgccattgacctcatcgagtacctcttgcgccgggtcgacatatcccaccagcggcgagtgggaaagacagtgaagataagcgtgcatatcaggctaccttttcgacagaaacgtcggcatcagggcgggtcagcgtcatcaccgacgccagtttattcagcgtattacccagggtttgcggaatggcggtggcggcaacaaagcgatccgggcgcatcaccaccagcgaagcattatgttgtgcgaaccagctacgcaggcgaccttgcgtatcccccacgcgtagtacgccgtcgtggttatcctgtgcggtatgaatttgcacttccggcaccacctgaatgaagcgggtgcccaacgcgcgccactgctggatttgctcgtcgctcatcccccacagtggattgcatccccagccaattaccgcgaagttcgcgccgatcgcgttatcgagcagcgtcacgtcgccgttttccagcgtgactttcggctgaataaacatcttgccgaccggagagtgcttcgcttcgccctctcgcaccagcgcaccgccgtaatattgcggcatcggcttgaagcgcatttcgaggaagtagcgttttactggcggcagataattcaacagccaggaaacgccgtcacgtaacgtaccctgccagcgtttcggcggagccagcacgttgcccgccgtcacggacaggtcaatcatcgctttggcgtgatcgcgtcgttcttgttgataggtatcgagcagcgcatcacgggctttcccctggataaccaacgccagtttccaggcgaggttaaaggcgtcgcgcataccgctgttatagccctgcccctgccataccggcatgatgtgcgcggcatcgcccgccagcagtacgcgatcaatacggaaacgttgcgccagtcgcgcgttgtgggtgtagacacgctggcgaatcaattcaacattgtccggattaggcagcactttgcttaacagcttgcgcatattttgcggctcacgcagctgttcttcggtttctcctggcatcaccataaattcaaagcgacgtaccgcatgaggtagcgcggcagaaacatacgggcgcaccggatcgcaacacaaatagatatgcggcgtacttaacggatcgttggcgatatctaccacaatccactgatttggcgcagttttaccttcaaacggcacattcagggtgcgacggacaaaacttgccccaccatcacaggccaccagccactgggctttgactatttcccgctgcccttctgccgttttcaggtgcaaggtcacttcgtcatcttgctgactgaaggcctccagctcgcgggaaaacaagcagcgcacattcggaaaacgcgacaccccttccagcatcaccgcatcgacctgcggctgaataaaggcgttacggcgcggccagccaaattcatcggtcattggctgaatatcagcaaaacagcggcctttcggggtgagaaaacgcatcgcgtgccacggcgtagtgtgcggcagaacatcatcgaccaggccgaccgactgcatggtgcgcagcgcctcgtcatcaataccaatcgcacgcgggtagtcgatcaacttatcgagtttctccaccaccagcacgtcaatgcccatctggccgagatagttcgccatcatcagcccaaccgggccggcaccagcgatcgccacctgaacgctatggttaacagcaggctggatgtcagggtgttgtattgccatttcagtacctcacgactcggacaaaatgtcgttgcgcgcacagtacagcgcaacttattttgttaaaaacatgtaaatgattttttattgtgcgctcagtataggaagggtgttttcggctacaatcaaaacatgcccgaatgtgcaccaggtgcaccacgttgttttaactatagaaatgtcaattaatatgcagaacaatgagcagacggaatacaaaaccgtgcgcggcttaacccgcggtctaatgttattaaatatgttaaataaacttgatggcggtgccagcgtcgggctgctggcggaactcagcggcctgcatcgcaccactgtgcggcgactgctggagacgctgcaggaagagggatatgtccgccgtagcccctccgatgatagttttcgactgaccatcaaagtgcggcaattaagcgaaggatttcgtgacgaacagtggatttctgcactggcggccccactgctgggcgatctgttgcgcgaagtggtatggccgacagatgtgtccacgctggatgttgatgcaatggtggtacgcgaaaccactcaccgtttcagccgcttatcctttcaccgggcaatggtcgggcgacgtttgccgcttctgaaaaccgcctcgggcctgacctggctggccttttgcccggaacaagaccgcaaggaattaatcgaaatgttagcctcccgccccggtgatgactatcaactggcacgggaaccgttaaagctggaagccattctggcgcgcgcgcgcaaagagggttacggacagaactaccgcggctgggatcaggaggagaagatcgcctctatcgccgtaccgctgcgcagtgaacaacgggtgattggctgtctgaatctggtgtatatggcgagcgcaatgaccattgaacaggcagcggaaaagcatcttccggcgctacaacgggtagcaaaacagatcgaagaaggggttgaatcgcaggctattctggtggccggaaggcgaagcggcatgcatttacgttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaactaggcaccccaggctttacactttatgcttccggctggtataatgtgtggacctgtagaaataattttgtttaactttaataaggagatataccatgggaggcagtactgttgcaccaactacaccgttggcaaccggcggtgcgctccgcaaagtgcgacaagccgtctttcccatctacggaaaccaagaagtcaccaaatttctgctcatcggatccattaaattctttataatcttggcactcacgctcacgcgtgataccaaggacacgttgattgtcacgcaatgtggtgccgaagcgattgcctttctcaaaatatacggggtgctacccgcagcgaccgcatttatcgcgctctattccaaaatgtccaacgccatgggcaaaaaaatgctattttattccacttgcattcctttctttacctttttcgggctgtttgatgttttcatttacccgaacgcggagcgactgcaccctagtttggaagccgtgcaggcaattctcccgggcggtgccgcatctggcggcatggcggttctggccaagattgcgacacactggacatcggccttattttacgtcatggcggaaatatattcttccgtatcggtggggctattgttttggcagtttgcgaacgacgtcgtcaacgtggatcaggccaagcgcttttatccattatttgctcaaatgagtggcctcgctccagttttagcgggccagtatgtggtacggtttgccagcaaagcggtcaactttgaggcatccatgcatcgactcacggcggccgtaacatttgctggtattatgatttgcatcttttaccaactcagttcgtcatatgtggagcgaacggaatcagcaaagccagcggcagataacgagcagtctatcaaaccgaaaaagaagaaacccaaaatgtccatggttgaatcggggaaatttctcgcgtcaagtcagtacctgcgtctaattgccatgctggtgctgggatacggcctcagtattaactttaccgaaatcatgtggaaaagcttggtgaagaaacaatatccagacccgctagattatcaacgatttatgggtaacttctcgtcagcggttggtttgagcacatgcattgttattttcttcggtgtgcacgtgatccgtttgttggggtggaaagtcggagcgttggctacacctgggatcatggccattctagcgttacccttttttgcttgcattttgttgggtttggatagtccagcacgattggagatcgccgtaatctttggaacaattcagagtttgctgagcaaaacctccaagtatgcccttttcgaccctaccacacaaatggcttatattcctctggacgacgaatcaaaggtcaaaggaaaagcggcaattgatgttttgggatcgcggattggcaagagtggaggctcactgatccagcagggcttggtctttgtttttggaaatatcattaatgccgcacctgtagtaggggttgtctactacagtgtccttgttgcgtggatgagcgcagctggccgactaagtgggctttttcaagcacaaacagaaatggataaggccgacaaaatggaggcaaagaccaacaaagaaaagtagttaacctaggctgctgccaccgctgagcaataagactcctgttgatagatccagtaatgacctcagaactccatctggatttgttcagaacgctcggttgccgccgggcgttttttattggtgagaat

SEQ ID NO: 6 - DNA, 125 bp, Escherichia coli
  source 1..125: mol_type = genomic DNA
aggagtaaaaatgattccggggatccgtcgacctgcagttcgaagttcctattctctagaaagtataggaacttcgaagcagctccagcctacagaaactaacgaagatttttaatcgtcttgtt

SEQ ID NO: 7 - DNA, 1082 bp, Escherichia coli
  source 1..1082: mol_type = genomic DNA
aggagtaaaaatggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgataacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcgccgtttaaacaggctgaattccagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcgactgcctggctgaaagataacccggaaaccgcgaaagagatcgagaagaaagtacgtgagttgctgctgagcaacccgaactcaacgccggatttctctgtagatgatagcgaaggcgtagcagaaactaacgaagatttttaatcgtcttgtt

SEQ ID NO: 8 - protein, 783 aa, Escherichia coli
MAQAGFILTRHWRDTPQGTEVSFWLATDNGPLQVTLAPQESVAFIPADQVPRAQHILQGEQGVRLTPLALKDFHRQPVYGLYCRAHRQLMNYEKRLREGGVTVYEADVRPPERYLMERFITSPVWVEGDMHNGAIVNARLKPHPDYRPPLKWVSIDIETTRHGELYCIGLEGCGQRIVYMLGPENGDASALDFELEYVASRPQLLEKLNAWFANYDPDVIIGWNVVQFDLRMLQKHAERYRIPLRLGRDNSELEWREHGFKNGVFFAQAKGRLIIDGIEALKSAFWNFSSFSLETVAQELLGEGKSIDNPWDRMDEIDRRFAEDKPALATYNLKDCELVTQIFHKTEIMPFLLERATVNGLPVDRHGGSVAAFGHLYFPRMHRAGYVAPNLGEVPPHASPGGYVMDSRPGLYDSVLVLDYKSLYPSIIRTFLIDPVGLVEGMAQPDPEHSTEGFLDAWFSREKHCLPEIVTNIWHGRDEAKRQGNKPLSQALKIIMNAFYGVLGTTACRFFDPRLASSITMRGHQIMRQTKALIEAQGYDVIYGDTDSTFVWLKGAHSEEEAAKIGRALVQHVNAWWAETLQKQRLTSALELEYETHFCRFLMPTIRGADTGSKKRYAGLIQEGDKQRMVFKGLETVRTDWTPLAQQFQQELYLRIFRNEPYQEYVRETIDKLMAGELDARLVYRKRLRRPLSEYQRNVPPHVRAARLADEENQKRGRPLQYQNRGTIKYVWTTNGPEPLDYQRSPLDYEHYLTRQLQPVAEGILPFIEDNFATLMTGQLGLF

SEQ ID NO: 9 - protein, 320 aa, Escherichia coli
MAISSRNTLLAALAFIAFQAQAVNVTVAYQTSAEPAKVAQADNTFAKESGATVDWRKFDSGASIVRALASGDVQIGNLGSSPLAVAASQQVPIEVFLLASKLGNSEALVVKKTISKPEDLIGKRIAVPFISTTHYSLLAALKHWGIKPGQVEIVNLQPPAIIAAWQRGDIDGAYVWAPAVNALEKDGKVLTDSEQVGQWGAPTLDVWVVRKDFAEKHPEVVKAFAKSAIDAQQPYIANPDAWLKQPENISKLARLSGVPEGDVPGLVKGNTYLTPQQQTAELTGPVNKAIIDTAQFLKEQGKVPAVANDYSQYVTSRFVQ

SEQ ID NO: 10 - protein, 91 aa, Escherichia coli
MASVSISCPSCSATDGVVRNGKSTAGHQRYLCSHCRKTWQLQFTYTASQPGTHQKIIDMAMNGVGCRATARIMGVGLNTILRHLKNSGRSR

SEQ ID NO: 11 - protein, 167 aa, Escherichia coli
MPGNCPHYGRWPQHDFTSLKKLRPQSVTSRIQPGSDVIVCAEMDEQWGYVGAKSRQRWLFYAYDRLRKTVVAHVFGERTMATLGRLMSLMSPFDVVIWMTDGWPLYESRLKGKLHVISKRYTQRIERHNLNLRQHLARLGRKSLSFSKSVELHDKVIGHYLNIKHYQ

SEQ ID NO: 12 - protein, 511 aa, Escherichia coli
MGGSTVAPTTPLATGGALRKVRQAVFPIYGNQEVTKFLLIGSIKFFIILALTLTRDTKDTLIVTQCGAEAIAFLKIYGVLPAATAFIALYSKMSNAMGKKMLFYSTCIPFFTFFGLFDVFIYPNAERLHPSLEAVQAILPGGAASGGMAVLAKIATHWTSALFYVMAEIYSSVSVGLLFWQFANDVVNVDQAKRFYPLFAQMSGLAPVLAGQYVVRFASKAVNFEASMHRLTAAVTFAGIMICIFYQLSSSYVERTESAKPAADNEQSIKPKKKKPKMSMVESGKFLASSQYLRLIAMLVLGYGLSINFTEIMWKSLVKKQYPDPLDYQRFMGNFSSAVGLSTCIVIFFGVHVIRLLGWKVGALATPGIMAILALPFFACILLGLDSPARLEIAVIFGTIQSLLSKTSKYALFDPTTQMAYIPLDDESKVKGKAAIDVLGSRIGKSGGSLIQQGLVFVFGNIINAAPVVGVVYYSVLVAWMSAAGRLSGLFQAQTEMDKADKMEAKTNKEK

SEQ ID NO: 13 - protein, 257 aa, Escherichia coli
MIKRTLLAAAIFSALPAYAGLTSITAGYDFTDYSGDHGNRNLAYAELVAKVENATLLFNLSQGRRDYETEHFNATRGQGAVWYKWNNWLTTRTGIAFADNTPVFARQDFRQDINLALLPKTLFTTGYRYTKYYDDVEVDAWQGGVSLYTGPVITSYRYTHYDSSDAGGSYSNMISVRLNDPRGTGYTQLWLSRGTGAYTYDWTPETRYGSMKSVSLQRIQPLTEQLNLGLTAGKVWYDTPTDDYNGLQLAAHLTWKF

SEQ ID NO: 14 - protein, 91 aa, Escherichia coli [sequence omitted]

SEQ ID NO: 15 - protein, 369 aa, Escherichia coli
MKSRAAVAFAPGKPLEIVEIDVAPPKKGEVLIKVTHTGVCHTDAFTLSGDDPEGVFPVVLGHEGAGVVVEVGEGVTSVKPGDHVIPLYTAECGECEFCRSGKTNLCVAVRETQGKGLMPDGTTRFSYNGQPLYHYMGCSTFSEYTVVAEVSLAKINPEANHEHVCLLGCGVTTGIGAVHNTAKVQPGDSVAVFGLGAIGLAVVQGARQAKAGRIIAIDTNPKKFDLARRFGATDCINPNDYDKPIKDVLLDINKWGIDHTFECIGNVNVMRAALESAHRGWGQSVIIGVAGAGQEISTRPFQLVTGRVWKGSAFGGVKGRSQLPGMVEDAMKGDIDLEPFVTHTMSLDEINDAFDLMHEGKSIRTVIRY

SEQ ID NO: 16 - protein, 277 aa, Escherichia coli
MELIEKHASFGGWQNVYRHYSQSLKCEMNVGVYLPPKAANEKLPVLYWLSGLTCNEQNFITKSGMQRYAAEHNIIVVAPDTSPRGSHVADADRYDLGQGAGFYLNATQAPWNEHYKMYDYIRNELPDLVMHHFPATAKKSISGHSMGGLGALVLALRNPDEYVSVSAFSPIVSPSQVPWGQQAFAAYLAENKDAWLDYDPVSLISQGQRVAEIMVDQGLSDDFYAEQLRTPNLEKICQEMNIKTLIRYHEGYDHSYYFVSSFIGEHIAYHANKLNMR

SEQ ID NO: 17 - protein, 179 aa, Escherichia coli
MAKLTLQEQLLKAGLVTSKKAAKVERTAKKSRVQAREARAAVEENKKAQLERDKQLSEQQKQAALAKEYKAQVKQLIEMNRITIANGDIGFNFTDGNLIKKIFVDKLTQAQLINGRLAIARLLVDNNSEGEYAIIPASVADKIAQRDASSIVLHSALSAEEQDEDDPYADFKVPDDLMW

SEQ ID NO: 18 - protein, 403 aa, Escherichia coli
MSTRTPSSSSSRLMLTIGLCFLVALMEGLDLQAAGIAAGGIAQAFALDKMQMGWIFSAGILGLLPGALVGGMLADRYGRKRILIGSVALFGLFSLATAIAWDFPSLVFARLMTGVGLGAALPNLIALTSEAAGPRFRGTAVSLMYCGVPIGAALAATLGFAGANLAWQTVFWVGGVVPLILVPLLMRWLPESAVFAGEKQSAPPLRALFAPETATATLLLWLCYFFTLLVVYMLINWLPLLLVEQGFQPSQAAGVMFALQMGAASGTLMLGALMDKLRPVTMSLLIYSGMLASLLALGTVSSFNGMLLAGFVAGLFATGGQSVLYALAPLFYSSQIRATGVGTAVAVGRLGAMSGPLLAGKMLALGTGTVGVMAASAPGILVAGLAVFILMSRRSRIQPCADA

SEQ ID NO: 19 - protein, 337 aa, Escherichia coli
MNGKKLYISDVTLRDGMHAIRHQYSLENVRQIAKALDDAHVDSIEVAHGDGLQGSSFNYGFGAHSDLEWIEAAADVVKHAKIATLLLPGIGTIHDLKNAWQAGARVVRVATHCTEADVSAQHIQYARELGMDTVGFLMMSHMTTPENLAKQAKLMEGYGATCIYVVDSGGAMNMSDIRDRFRALKAVLKPETQTGMHAHHNLSLGVANSIEAVEEGCDRIDASLAGMGAGAGNAPLEVFIAAADKLGWQHGTDLYALMDAADDLVRPLQDRPVRVDRETLALGYAGVYSSFLRHCETAAARYGLSAVDILVELGKRRMVGGQEDMIVDVALDLRNNK

SEQ ID NO: 20 - protein, 316 aa, Escherichia coli
MSKRKVAIIGSGNIGTDLMIKILRHGQHLEMAVMVGIDPQSDGLARARRMGVATTHEGVIGLMNMPEFADIDIVFDATSAGAHVKNDAALREAKPDIRLIDLTPAAIGPYCVPVVNLEANVDQLNVNMVTCGGQATIPMVAAVSRVARVHYAEIIASIASKSAGPGTRANIDEFTETTSRAIEVVGGAAKGKAIIVLNPAEPPLMMRDTVYVLSDEASQDDIEASINEMAEAVQAYVPGYRLKQRVQFEVIPQDKPVNLPGVGQFSGLKTAVWLEVEGAAHYLPAYAGNLDIMTSSALATAEKMAQSLARKAGEAA

SEQ ID NO: 21 - protein, 269 aa, Escherichia coli
MTKHTLEQLAADLRRAAEQGEAIAPLRDLIGIDNAEAAYAIQHINVQYDVAQGRRVVGRKVGLTHPKVQQQLGVDQPDFGTLFADMCYGDNEIIPFSRVLQPRIEAEIALVLNRDLPATDITFDELYNAIEWVLPALEVVGSRIRDWSIQFVDTVADNASCGVYVIGGPAQRPAGLDLKNCAMKMTRNNEEVSSGRGSECLGHPLNAAVWLARKMASLGEPLRTGDIILTGALGPMVAVNAGDRFEAHIEGIGSVAATFSSAAPKGSLS

SEQ ID NO: 22 - protein, 293 aa, Escherichia coli
MQEKMMSYQPQTEAATSRFLNVEEAGKTLRIHFNDCGQGDETVVLLHGSGPGATGWANFSRNIDPLVEAGYRVILLDCPGWGKSDSIVNSGSRSDLNARILKSVVDQLDIAKIHLLGNSMGGHSSVAFTLNWPERVGKLVLMGGGTGGMSLFTPMPTEGIKRLNQLYRQPTIENLKLMMDIFVFDTSDLTDALFEARLNNMLSRRDHLENFVKSLEANPKQFPDFGPRLAEIKAQTLIVWGRNDRFVPMDAGLRLLSGIAGSELHIFRDCGHWAQWEHADAFNQLVLNFLARP

SEQ ID NO: 23 - protein, 314 aa, Escherichia coli
MHAYLHCLSHSPLVGYVDPAQEVLDEVNGVIASARERIAAFSPELVVLFAPDHYNGFFYDVMPPFCLGVGATAIGDFGSAAGELPVPVELAEACAHAVMKSGIDLAVSYCMQVDHGFAQPLEFLLGGLDKVPVLPVFINGVATPLPGFQRTRMLGEAIGRFTSTLNKRVLFLGSGGLSHQPPVPELAKADAHMRDRLLGSGKDLPASERELRQQRVISAAEKFVEDQRTLHPLNPIWDNQFMTLLEQGRIQELDAVSNEELSAIAGKSTHEIKTWVAAFAAISAFGNWRSEGRYYRPIPEWIAGFGSLSARTEN

SEQ ID NO: 24 - protein, 554 aa, Escherichia coli
MAIQHPDIQPAVNHSVQVAIAGAGPVGLMMANYLGQMGIDVLVVEKLDKLIDYPRAIGIDDEALRTMQSVGLVDDVLPHTTPWHAMRFLTPKGRCFADIQPMTDEFGWPRRNAFIQPQVDAVMLEGVSRFPNVRCLFSRELEAFSQQDDEVTLHLKTAEGQREIVKAQWLVACDGGASFVRRTLNVPFEGKTAPNQWIVVDIANDPLSTPHIYLCCDPVRPYVSAALPHAVRRFEFMVMPGETEEQLREPQNMRKLLSKVLPNPDNVELIRQRVYTHNARLAQRFRIDRVLLAGDAAHIMPVWQGQGYNSGMRDAFNLAWKLALVIQGKARDALLDTYQQERRDHAKAMIDLSVTAGNVLAPPKRWQGTLRDGVSWLLNYLPPVKRYFLEMRFKPMPQYYGGALVREGEAKHSPVGKMFIQPKVTLENGDVTLLDNAIGANFAVIGWGCNPLWGMSDEQIQQWRALGTRFIQVVPEVQIHTAQDNHDGVLRVGDTQGRLRSWFAQHNASLVVMRPDRFVAATAIPQTLGNTLNKLASVMTLTRPDADVSVEKVA

SEQ ID NO: 25 - protein, 315 aa, Escherichia coli
MIFYCALSIGRVFSATIKTCPNVHQVHHVVLTIEMSINMQNNEQTEYKTVRGLTRGLMLLNMLNKLDGGASVGLLAELSGLHRTTVRRLLETLQEEGYVRRSPSDDSFRLTIKVRQLSEGFRDEQWISALAAPLLGDLLREVVWPTDVSTLDVDAMVVRETTHRFSRLSFHRAMVGRRLPLLKTASGLTWLAFCPEQDRKELIEMLASRPGDDYQLAREPLKLEAILARARKEGYGQNYRGWDQEEKIASIAVPLRSEQRVIGCLNLVYMASAMTIEQAAEKHLPALQRVAKQIEEGVESQAILVAGRRSGMHLR

SEQ ID NO: 26 - protein, 360 aa, Escherichia coli
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 27 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 28 - DNA, 49 bp, synthetic construct
  misc_feature 1..49: note = Description of Artificial Sequence: Synthetic oligonucleotide
  source 1..49: mol_type = other DNA
ctgtataaaaccacagccaatcaaacgaaaccaggctatactcaagcct

SEQ ID NO: 29 - DNA, 49 bp, synthetic construct
  misc_feature 1..49: note = Description of Artificial Sequence: Synthetic oligonucleotide
  source 1..49: mol_type = other DNA
ttttataaatacacagccaatcaaacgaaaccaggctatactcaagcct

SEQ ID NO: 30 - DNA, 133 bp, synthetic construct
  misc_feature 1..133: note = Description of Artificial Sequence: Synthetic polynucleotide
  source 1..133: mol_type = other DNA
ctcagcggtggcagcagcctaggttaactacttttctttgttggtctttgcctccattttgtcggcctggtaatgactccaacttattgatagtgttttatgttcagataatgcccgatgactttgtcatgca

SEQ ID NO: 31 - DNA, 133 bp, synthetic construct
  misc_feature 1..133: note = Description of Artificial Sequence: Synthetic polynucleotide
  variation 22: note = a, c, t, g, unknown or other
  source 1..133: mol_type = other DNA
ctcagcggtggcagcagcctangttaatcatttttctttgttggttttggcttccattttatctgcttggtaatgactccaacttattgatagtgttttatgttcagataatgcccgatgactttgtcatgca

SEQ ID NO: 32 - DNA, 133 bp, synthetic construct
  misc_feature 1..133: note = Description of Artificial Sequence: Synthetic polynucleotide
  source 1..133: mol_type = other DNA
ctcagcggtggcagcagcctaggttaatcatttttctttgttggttttggcttccattttatctgcttggtaatgactccaacttattgatagtgttttatgttcagataatgcccgatgactttgtcatgca